(19) United States
(12) Patent Application Publication
(10) Pub. No.: US 2006/0234909 A1
(43) Pub. Date: Oct. 19, 2006

(54) Compositions and methods for increasing drug efficiency

(76) Inventors: Michael James Newman, San Diego, CA (US); Angelo John Castellino, San Diego, CA (US); Joel Desharnais, San Diego, CA (US); Zijian Guo, San Diego, CA (US); Qing Li, San Diego, CA (US); Carlo Ballatore, San Diego, CA (US); Chengzao Sun, San Diego, CA (US)

Correspondence Address: JONES DAY, 222 EAST 41ST ST, NEW YORK, NY, 10017, US

(21) Appl. No.: 11/376,695

(22) Filed: Mar. 14, 2006

Related U.S. Application Data
(63) Continuation of application No. 10/948,707, filed on Sep. 22, 2004, now abandoned.
(60) Provisional application No. 60/505,325, filed on Sep. 22, 2003.
(60) Provisional application No. 60/568,340, filed on May 4, 2004.
(60) Provisional application No. 60/581,835, filed on Jun. 22, 2004.

(51) Int. Cl. A61K 38/17; A61K 38/16

(52) U.S. Cl. 514/2; 514/8; 530/395; 530/409

(57) ABSTRACT

In one embodiment, provided herein are compositions and methods for increasing drug efficiency. In certain embodiments, the compositions contain conjugates having the formula:
D-L-S wherein D is a drug moiety; L, which may or may not be present, is a non-releasing linker moiety; and S is a substrate for a protein or lipid kinase that is overexpressed, overactive or exhibits undesired activity in a target system.

### RELATED APPLICATIONS

This application is a continuation of U.S. application Ser. No. 10/948,707, filed Sep. 22, 2004, to Newman et al., which claims benefit of priority under 35 U.S.C. §119(e) to U.S. provisional application Ser. No. 60/505,325, filed Sep. 22, 2003, to Newman et al., entitled “DRUG IMPROVEMENT BY PROTEIN KINASE SPECIFIC TARGETING AND TRAPPING”, U.S. provisional application Ser. No. 60/568,340, filed May 4, 2004, to Newman et al., entitled “COMPOSITIONS AND METHODS FOR INCREASING DRUG EFFICIENCY” and U.S. provisional application Ser. No. 60/581,835, filed Jun. 22, 2004, to Castellino et al., entitled “SMALL MOLECULE COMPOSITIONS AND METHODS FOR INCREASING DRUG EFFICIENCY USING COMPOSITIONS THEREOF”. The subject matter of the above-referenced applications are incorporated by reference in their entirety.

### SEQUENCE LISTING

This application includes a Sequence Listing submitted on compact disc. The Sequence Listing is recorded on two compact discs (CD-R), including one duplicate, containing Filename “11685-0009-999 Sequence Lisiting.txt” (ASCII TEXT) of file size 974,336 bytes. The Sequence Listing on compact disc(s) is incorporated by reference herein in its entirety.

1. Field

Conjugates, compositions and methods for improving drug efficiency are provided. The conjugates provided are for delivery of therapeutic agents for treating a variety of disorders, such as, proliferative diseases, autoimmune diseases, infectious diseases and inflammatory diseases. The conjugates contain therapeutic agents connected to substrates for protein or lipid kinases, optionally via a non-releasable linker.

2. Background

A wide variety of drugs have been used for treating conditions caused by undesirable chronic or aberrant cellular activation, migration, proliferation or survival (ACAMPS). ACAMPS-related conditions include, but are not limited to, cancer, chronic inflammation, autoimmune syndromes, transplant rejection and osteoporosis. However, the effectiveness of the drug is frequently limited by side effects produced in cells not directly involved in the genesis or maintenance of the condition being treated. Drug effectiveness can also be limited by active efflux of the drug as exemplified by the treatment of cancer wherein drug is actively removed from the treated cell by a P-glycoprotein transporter.

Significant limitations of drugs used to treat ACAMPS-related diseases result from their action upon cell types not involved with the disease. A common feature of all ACAMPS conditions has been found to involve signal transduction pathways utilizing protein kinases to initiate and amplify inter-, intra- and extracellular signals. Protein kinases engage in signal transduction by auto activation and activation of other proteins via phosphorylation on tyrosine, serine or threonine residues. Dysregulated phosphorylation-mediated signal amplification contributes directly to chronic or aberrant cellular activation, migration, proliferation and survival. Abnormally high levels of protein kinase activity can result from mutational activation of the kinase or transient overexpression of either the kinase or a kinase activator or downregulation or mutational deactivation of a kinase inhibitor.

Many attempts have been made to increase the effectiveness of ACAMPS drugs by prodrug and extracellular targeting approaches. Examples for the treatment of cancer with paclitaxel include conjugates prepared with polyethylene glycol (PEG) (Greenwald, R. B., et al., J. Med. Chem. (1996) 39:424-431), polyglutamate (PG) (Li, C., et al., Cancer Res. (1998) 58:2404-2409) and docosahexaenoic acid (DHA) (Bradley, M. O., et al., Clin. Cancer Res. (2001) 7:3229-3238) (Whelan, J., Drug Discov. Today (2002) 7:90-92, for review). In all cases the conjugate must be cleaved to produce the parent taxane, which is disadvantageous since the free drug is capable of diffusing out of the targeted cells and is susceptible to multidrug resistance (MDR).

Another approach for targeting to tumor cells involves conjugation of the drug to a peptide or antibody that recognizes a cell surface antigen or receptor. In one example, paclitaxel was targeted to tumor cells via conjugation with a 7-amino acid synthetic peptide that binds to the bombesin/gastrin-releasing peptide receptor (Safavy, A., et al., J. Med. Chem. (1999) 42:4919-4924). The conjugate retained receptor binding and was cleaved after internalization. Again, this approach depends on cleavage of a labile bond and release of the free drug inside the cell.

A cell surface targeting approach has also been attempted with EGF receptor antibodies given the established role of EGF receptor kinases in cancer. However, there was no improvement of in vivo efficacy beyond that obtained with the antibody alone (Safavy, A., et al., Bioconjug. Chem. (2003) 14:302-310).

Another approach involves antibody-mediated targeting, which has historically been difficult to achieve and presents many hurdles associated with protein and antibody drug development. Reliance on release of parent drug and the inefficiency of this release are considerable disadvantages. Furthermore the heterogeneous nature of tumor cells results in limited distribution of the receptors. Therefore, treatment by this approach will result in clonal selection of tumor cells lacking the cell surface marker leading to resistance. Additionally, susceptibility to MDR remains since the parent drug is released. An additional approach is based on the discovery of cell-penetrating peptide (CPP) sequences that cross cell membranes by an endocytic process. These peptides have been derived, for example, from Antennapedia homeodomain, HIV Tat and the antimicrobial peptide protegrin 1 (Thoren, P. E., et al., Biochem. Biophys. Res. Com (2003) 307:100-107, Vives, E., et al., Curr. Protein Pet. Sci. (2003) 4:125-133). These membrane permeant peptides are generally 16-18 amino acids in length and contain at least 5 to 7 positively charged arginine or lysine residues.

Several groups have attached CPP's to drugs (including anti-cancer agents), facilitating their uptake and retention in cells and their penetration across the blood brain barrier. However, the CPP approach does not provide any targeting functionality, and does not discriminate between cells-type responsible for the condition being treated and normal cell-types. Thus, there remains a need for compositions and methods for improving drug efficiency, particularly against ACAMPS-related conditions.

### SUMMARY

Provided herein are compounds and methods for targeted delivery of drugs. The compounds are conjugates that contain a drug moiety and a substrate for a protein kinase or a lipid kinase non-releasably linked thereto. The drug moieties include therapeutic agents, such as a cytotoxic agents, and diagnostic agents, such as labeled moieties and imaging agents. The substrates are substrates for a protein kinase or a lipid kinase. In certain embodiments, the drug moiety is a therapeutic agent. In certain embodiments, the drug moiety is a labeling agent.

The conjugates contain one or more substrates for one or a plurality of protein kinases or lipid kinases non-releasably linked thereto, either directly or via a non-releasing linker to a drug moiety, such as a cytotoxic agent. The conjugates provided herein contain the following components: (substrate)ₜ, (linker)_{q}, and (drug)_{d} in which: at least one substrate for a protein kinase or a lipid kinase is non-releasably linked, optinally via a linker, to a drug moiety. t is 1 to 6 and each substrate is the same or different, and is generally 1 or 2; q is 0 to 6; 0 to 4; 0 or 1; d is 1 to 6, in certain embodiment 1 or 2 and each drug moieties are the same or different; linker refers to any non-releasing linker; and the drug is any a therapeutic agent, such as a cytotoxic agent, including an anti-cancer drug, a diagnostic agent, such as an imaging agent or labeled moiety. The drug moiety of the drug conjugate may be derived from a naturally occurring or synthetic compound that may be obtained from a wide variety of sources, including libraries of synthetic or natural compounds. Exemplary drug moieties can be cytotoxic agents, including, but not limited to, anti-infective agents, antihelminthic, antiprotozoal agents, antimalarial agents, antiamebic agents, antileiscmanial drugs, antitrichomonal agents, antitrypanosomal agents, sulfonamides, antimycobacterial drugs, or antiviral chemotherapeutics.

In one embodiment, the conjugates for use in the compositions and methods provided herein have formula (1):
(D)_{d}-(L)_{q}-(S)ₜ (1)
or a derivative thereof, wherein D is a drug moiety; d is 1 to 6, or is 1 or 2; L is a non-releasing linker; q is 0 to 6, or is 0 to 4, or is 0 or 1; S is a substrate for a protein kinase or a lipid kinase; and t is 1 to 6, or is 1 or 2, or is 1. In the conjugates, the drug moiety is covalently attached, optionally via a non-releasing linker, to the substrate. In the conjugates provided herein, the conjugation of the drug moiety(s) or non-releasing linker linked thereto can be at various positions of the substrate.

In the conjugates that contain two drug moieties, which are the same or different, conjugation to the drug moiety(s) or non-releasing linker linked thereto can be at various positions of the substrate.

In certain embodiments, the kinase is overexpressed, overactive or exhibits undesired activity in a target system. The action of the kinase on the substrate results in a negative charge on the conjugate. The action of the kinase on the substrate may result in improved drug efficiency.

The target system may be a cell, tissue or organ. In particular embodiments, the cell is a tumor cell or a tumor-associated endothelial cell. The target system may also be associated with cancer, inflammation, angiogenesis, autoimmune syndromes, transplant rejection or osteoporosis.

In another embodiment, conjugates for use in compositions and methods for increasing drug efficiency are provided. Also provided are methods for treating conditions caused by undesirable chronic or aberrant cellular activation, migration, proliferation or survival (ACAMPS). In one embodiment, the methods are for ameliorating a cell-proliferative disorder, including cancer.

In certain embodiments, the conjugates have formula (2)
D-L-Sp (2)
wherein D and L are as defined in formula (1); and

Sp is a substrate for a protein kinase. Examples of protein kinases include, but are not limited to, AFK, Akt, AMP—PK, Aurora kinase, beta-ARK, Abl, ATM, Auro kinase, ATR, CAK, Cam-II, Cam-III, CCD, Cdc2, Cdc28-dep, CDK, Flt, Fms, Hck, CKI, CKII, Met, DnaK, DNA-PK, Ds-DNA, EGF—R, ERA, ERK, ERT, FAK, FES, FGR, FGF—R, Fyn, Gag-fps, GRK, GRK2, GRK5, GSK, H4-PK-1, IGF—R, IKK, INS—R, JAK, KDR, Kit, Lck, MAPK, MAPKKK, MAPKAP2, MEK, MEK, MFPK, MHCK, MLCK, p135tyk2, p37, p38, p70S6, p74Raf-1, PDGF—R, PD, PhK, PI3K, PKA, PKC, PKG, Raf, PhK, RS, SAPK, Src, Tie-2, m-TOR, TrkA, VEGF—R, YES, or ZAP-70. In particular embodiments, the kinase is Akt, Abl, CAK, Cdc2, Fms, Met, EGF—R, ERK1, ERK2, FAK, Fyn, IGF—R, Lck, p70S6, PDGF−R, PI3K, PKA, PKC, Raf, Src, Tie-2 or VEGF—R. In one example, the kinase is VEGF—R2 (KDR).

In certain embodiments, the conjugates have formula (3)
D-L-S1 (3)
wherein D and L are as defined in formula (1); and

S1 is a substrate for a lipid kinase. Examples of lipid kinases include, but are not limited to, phosphoinositol kinase, diacylglycerol kinase and sphingosine kinase.

The substrate, in certain embodiments, is phosphorylated upon action of a kinase such as Akt, Abl, CAK, Cdc2, Fms, Met, EGF—R, ERK1, ERK2, FAK, Fyn, IGF—R, Lck, p70S6, PDGF—R, PI3K, PKA, PKC, Raf, Src, Tie-2, VEGF—R or sphingosine kinase. In the above formula 1, the drug moiety can be a hydrophobic drug. In certain embodiments, D can be a detectable label. In certain embodiments, the drug is an anti-cancer drug.

Pharmaceutical compositions containing a conjugate provided herein and a pharmaceutically acceptable carrier are provided.

Also provided are methods for using the conjugates. The methods provided are methods for treating conditions caused by undesirable chronic or aberrant cellular activation, migration, proliferation or survival (ACAMPS). Furthermore, methods for ameliorating a cell-proliferative disorder including, but not limited to, cancer are also provided. In one embodiment, the conjugates are for use in methods for treating cancer.

Also provided are methods of improving drug efficiency by administering a therapeutically effective amount of a conjugate provided herein to a cell, tissue, organ or organism, wherein the action of the kinase on the substrate results in improved drug efficiency.

In one embodiment, methods for identifying kinase substrates capable of selectively accumulating in a target system are provided. The methods contain the steps of: a) contacting one or more conjugates with a kinase that is overexpressed, overactive or exhibits undesired activity in a target system; and b) determining kinase activity on one or more conjugates. In other embodiments, the method for identifying kinase substrates capable of selectively accumulating in a target system further contains the steps of: c) determining a first amount or a plurality of first amounts of one or more conjugates in the target system; and d) determining a second amount or a plurality of second amounts of one or more conjugates in a non-target system.

In one example, one or more conjugates may contain a detectable label. For example, the label may be radioactive or fluorescent.

The target system may be associated with cancer, inflammation, angiogenesis, autoimmune syndromes, transplant rejection or osteoporosis. The target system may be a cell, tissue or organ. In one embodiment, the cell may be a tumor cell or a tumor-associated endothelial cell.

In one embodiment, methods for identifying conjugates capable of exhibiting selective toxicity against a target system are provided. The methods contain the steps of: a) contacting one or more conjugates containing a drug moiety with a target system; and b) determining the cytotoxicity of the one or more conjugates against the target system.

### DETAILED DESCRIPTION OF EMBODIMENTS

A. Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

The singular forms “a,” “an,” and “the” include plural references, unless the context clearly dictates otherwise. Thus, for example, references to a composition for delivering “a drug” include reference to one, two or more drugs.

As used herein, “drug conjugate” or a “conjugate” refers to compounds having one or more drug moieties non-releasably linked, optionally via a non-releasable linker, to a substrate for a protein kinase or a lipid kinase.

The term “protein kinase” as used herein is intended to include all enzymes which phosphorylate an amino acid residue within a protein or peptide. In certain embodiments, protein kinases for use herein include protein-serine/threonine specific protein kinases, protein-tyrosine specific kinases and dual-specificity kinase. Other protein kinases which can be used herein include protein-cysteine specific kinases, protein-histidine specific kinases, protein-lysine specific kinases, protein-aspartic acid specific kinases and protein-glutamic acid specific kinases. A protein kinase used herein can be a purified native protein kinase, for example purified from a biological source. Some purified protein kinases are commercially available (e.g., protein kinase A from Sigma Chemical Co.). Alternatively, a protein kinase used in the method of the invention can be a recombinantly produced protein kinase. Many protein kinases have been molecularly cloned and characterized and thus can be expressed recombinantly by standard techniques. A recombinantly produced protein kinase which maintains proper kinase function can be used herein. If the recombinant protein kinase to be examined is a eukaryotic protein kinase, in one embodiment, the protein kinase is recombinantly expressed in a eukaryotic expression system to ensure proper post-translational modification of the protein kinase. Many eukaryotic expression systems (e.g., baculovirus and yeast expression systems) are known in the art and standard procedures can be used to express a protein kinase recombinantly. A recombinantly produced protein kinase can also be a fusion protein (i.e., composed of the protein kinase and a second protein or peptide, for example a protein kinase fused to glutathione-S-transferase (GST)) as long as the fusion protein retains the catalytic activity of the non-fused form of the protein kinase. Furthermore, the term “protein kinase” is intended to include portions of native protein kinases which retain catalytic activity. For example, a subunit of a multisubunit kinase which contains the catalytic domain of the protein kinase can be used in the method of the invention.

As used herein the term “lipid kinase” is intended to include all enzymes which phosphorylate a lipid residue. In certain embodiments, lipid kinases for use herein include sphingosine kinase.

As used herein, “substrate” is a molecule which is subject to phosphorylation by a protein kinase or a lipid kinase, and encompasses species which can be converted by chemical and/or enzymatic reaction(s) to a substrate upon or after introduction of the molecule (in conjugate form) to a cell, tissue, organ or organism. The substrate contains at least one residue that can be phosphorylated by a protein kinase or a lipid kinase. In certain embodiments, the phosphorylation site is capped with a suitable capping group. In such cases, the capping group is removed under physiological conditions before the substrate is phosphorylated. In other embodiments, the residue adjucent to the site of phosphorylation can be masked thereby blocking the action of the kinase. In such cases, removal of the masking group is required to induce phosphorylation of the substrate. The substrates for use herein include, but are not limited to substrates for protein kinases such as Akt, Abl, CAK, Cdc2, Fms, Met, EGF—R, ERK1, ERK2, FAK, Fyn, IGF—R, Lck, p70S6, PDGF—R, PI3K, PKA, PKC, Raf, Src, Tie-2 and VEGF—R or substrates for lipid kinases such as sphingosine kinase.

The substrates for protein kinases include, but are not limited to, natural and non-natural peptides and their analogs, that can be phosphorylated by the particular protein kinase.

As used herein, “peptide” encompasses any peptide comprised of amino acids, amino acid analogs, peptidomimetics or combinations thereof. The term “amino acids” refers either to natural and/or unnatural synthetic amino acids, including both the D and L isomers, and encompasses any amine containing acid compound. In one embodiment, the peptides provided are between three to twenty units in length, containing up to four charged residues and are derived from the 20 naturally occurring species in D or L form. The peptide may contain modifications to the C-and/or N-terminus which include, but are not limited to, amidation or acetylation. In certain embodiments, the amino acid residues contain reactive side chains, for example carboxy side chain in glutamic acid, that can be capped by capping groups known in the art.

As used herein, “minimally charged peptide” refers to a peptide containing up to 4 charges, positive or negative. In one example, a positive charge is due to protonation of a basic amine nitrogen.

As used herein, “drug” or “drug moiety” is any drug or other agent that is intended for delivery to a targeted cell or tissue, such as cells or tissues associated with aberrant cellular activation, migration, proliferation or survival. Drug moiety for use herein, include, but are not limited to, anti-cancer agents, anti-angiogenic agents, cytotoxic agents and labeling agents, as described herein and known to those of skill in the art.

As used herein, an anti-cancer agent (used interchangeably with “anti-tumor or anti-neoplasm agent”) refers to any agents used in the treatment of cancer. These include any agents, when used alone or in combination with other compounds, that can alleviate, reduce, ameliorate, prevent, or place or maintain in a state of remission of clinical symptoms or diagnostic markers associated with neoplasm, tumor or cancer, and can be used in methods, combinations and compositions provided herein. Non-limiting examples of anti-neoplasm agents include anti-angiogenic agents, alkylating agents, antimetabolite, certain natural products that are anti-neoplasm agents, platinum coordination complexes, anthracenediones, substituted ureas, methylhydrazine derivatives, adrenocortical suppressants, certain hormones, antagonists and anti-cancer polysaccharides.

As used herein, anti-angiogenic agent refers to any compound, that, when used alone or in combination with other treatment or compounds, can alleviate, reduce, ameliorate, prevent, or place or maintain in a state of remission, one or more clinical symptoms or diagnostic markers associated with undesired and/or uncontrolled angiogenesis. Thus, for purposes herein an anti-angiogenic agent refers to an agent that inhibits the establishment or maintenance of vasculature. Such agents include, but are not limited to, anti-tumor agents, and agents for treatments of other disorders associated with undesirable angiogenesis, such as diabetic retinopathies, hyperproliferative disorders and others.

As used herein, “labeling agent” or “label” is a molecule that allows for the manipulation and/or detection of the conjugate which contains the label. Examples of labels include spectroscopic probes such as chromophores, fluorophores, and contrast agents. Other spectroscopic probes have magnetic or paramagnetic properties. The label may also be a radioactive molecule or a molecule that is part of a specific binding pair well known in the art such as biotin and streptavidin.

As used herein, “drug-linker construct” refers to a chemical combination wherein a drug moiety and a linker moiety are covalently attached. Similarly, a “drug-substrate construct” refers to a chemical combination wherein a drug moiety and a substrate moiety are covalently attached.

As used herein, “linker-substrate construct” refers to a chemical combination wherein a linker moiety and a substrate moiety are covalently attached.

As used herein, “hydrophobic drug” refers to any organic or inorganic compound or substance having biological or pharmaceutical activity with water solubility of less than 100 mg/ml, having a log P greater than 2, being lipid soluble or not adsorbing water.

As used herein, the term “effective amount of therapeutic response” refers to an amount which is effective in prolonging the survivability of the patient beyond the survivability in the absence of such treatment. Prolonging survivability also refers to improving the clinical disposition or physical well-being of the patient. When used in reference to cancer treatment methods, the term “therapeutically effective amount” refers to an amount which is effective, upon single or multiple dose administration to the patient, in controlling tumor growth. As used herein, “controlling tumor growth” refers to slowing, interrupting, arresting or stopping the migration or proliferation of tumor or tumor-associated endothelial cells.

The cytotoxic selectivity of the conjugates provided herein is assessed by comparing conjugate cytotoxicity against normal cells proliferating in monolayer to the conjugate cytotoxicity in the tumor cells proliferating in soft agar. In some embodiments, the conjugates show highter cytotoxicity selectivity for tumor cells as compared to the normal cells. As used herein, the term “cytotoxic selectivity index” refers to the ratio of EC₅₀ of the conjugate in tumor cells to the EC₅₀ of the conjugate in normal cell. In certain embodiments, the conjugates provided herein have higher cytotoxic selectivity for tumor cells than that of the parent drug. In certain embodiments, the conjugates provided herein show improved cytotoxic selectivity index as compared to the parent drug. The cytotoxic selectivity index for the conjugates provided herein are calculated by the methods provided herein.

As used herein, the term “improved drug efficiency” refers to a property of a drug within the conjugate which is improved relative to the drug in free form. Improved drug efficiency includes, but is not limited to, increased solubility, altered pharmacokinetics, including adsorption, distribution, metabolism and excretion, an increase in maximum tolerated dose, a reduction of side effects, an increase in cytotoxic selectivity index, an ability to surmount or avoid resistance mechanisms, or an ability to be administered chronically or more frequently. For example, a more efficient drug may have an improved cytotoxic selectivity index as compared to a less efficient drug. In certain embodiments, the improvement in the cytotoxic selectivity index is at least 1.5 fold greater is the conjugate.

As used herein, “non releasing linker moiety” or “non releasable linker moiety” refers to a linker moiety that is attached to a drug moiety through a covalent bond or functionality which remains substantially intact under physiological conditions during a period of time required for eliciting a pharmacological response such that the pharmacological response is not due to free drug. In some embodiments, the time is sufficient for uptake of the conjugate by the target system. In certain embodiments, the linkage remains from about 10% up to about 100% intact under physiologic conditions in a period of about 0.1 hours up to about 3 hours. In certain embodiments, the linker is more than 50% intact, in another embodiment, more than 60%, more than 70%, 80% or 90% intact. Evaluation of the stability of such linkage can be made by one of skill in the art using methods known in the art.

As used herein, “linker moiety” refers to the intervening atoms between the drug moiety and substrate. A linker precursor, used interchangeably with linker precursor moity, is a compound that is used in the synthesis of a drug linker construct or a substrate linker construct. The terms “linker” and “linking moiety” herein refer to any moiety that non-releasably connects the substrate moiety and drug moiety of the conjugate to one another. The linking moiety can be a covalent bond or a chemical functional group that directly connects the drug moiety to the substrate. The linking moiety can contain a series of covalently bonded atoms and their substituents which are collectively referred to as a linking group. Linking moieties are characterized by a first covalent bond or a chemical functional group that connects the drug moiety to a first end of the linker group and a second covalent bond or chemical functional group that connects the second end of the linker group to the substrate, in certain embodiments, to a carboxy terminus of a peptide substrate. The first and second functionality, which independently may or may not be present, and the linker group are collectively referred to as the linker moiety. The linker moiety is defined by the linking group, the first functionality if present and the second functionality if present. As used herein, the linker moiety contains atoms interposed between the drug moiety and substrate, independent of the source of these atoms and the reaction sequence used to synthesize the conjugate.

As used herein “non-releasably linked” refers to linkage of a drug moiety through a covalent bond or functionality wherein the linkage remains substantially intact under physiological conditions during a period of time required for eliciting a pharmacological response such that the pharmacological response is not due to free drug. In certain embodiments, the linkage remains from about 10% up to about 100% intact under physiologic conditions in a period of about 0.1 hours up to about 3 hours. In certain embodiments, the linker is more than 50% intact, in another embodiment, more than 60%, more than 70%, 80% or 90% intact.

In the conjugates provided herein, in certain embodiments, L′, L″ refers to the atoms or covalent bonds that connect the first and the second functionalities of the linker or the linking moiety.

As used herein, “an amino acid sequence motif for a phosphorylation site of a protein kinase” is intended to describe one or more amino acid sequences which represent a consensus sequence motif for the region including and surrounding an amino acid residue which is phosphorylated by a protein kinase. The methods for determining an amino acid sequence motif for the phosphorylation site of a protein kinase are known in the art (for example, see, U.S. Pat. No. 5,532,167) and involve contacting a protein kinase to be examined with an oriented degenerate peptide library composed of non-phosphorylated peptides having a phosphorylatable amino acid residue at a fixed non-degenerate position. For a given kinase, only a small subset of the peptides have amino acids surrounding the phosphorylatable residue that create a desired sequence for binding to the kinase and phosphorylation by the kinase. The protein kinase is allowed to phosphorylate the subset of peptides that are desired substrates for the kinase, thereby converting this population of peptides to a population of phosphorylated peptides. Next, the population of phosphorylated peptides is separated from the remaining non-phosphorylated peptides. Finally, the mixture of phosphorylated peptides is subjected to sequencing (e.g., automated sequencing) and the abundance of each amino acid determined at each cycle of sequencing is compared to the abundance of each amino acid at the same cycle in the starting peptide library. Since the phosphorylated residue is at the same position in every peptide of the library (e.g., residue 7 from the N-terminus), the most abundant amino acid(s) at a particular cycle indicate the amino acid(s) desired by the kinase at that position relative to the site of phosphorylation.

As used herein, the term “degenerate peptide library” refers to populations of peptides in which different amino acid residues are present at the same position in different peptides within the library. For example, a population of peptides of 10 amino acids in length in which the amino acid residue at position 5 of the peptides can be any one of the twenty amino acids would be a degenerate peptide library. A position within the peptides which is occupied by different amino acids in different peptides is referred to herein as a “degenerate position”; a position within the peptides which is occupied by the same amino acid in different peptides is referred to herein as a “non-degenerate position”. The “oriented degenerate peptide library” used in the methods for determining an amino acid sequence motif for the phosphorylation site of a protein kinase is composed of non-phosphorylated peptides which have a phosphorylatable amino acid residue at a fixed, non-degenerate position. This means that the peptides contained within the library all have the same phosphorylatable amino acid residue at the same position within the peptides. The term “phosphorylatable amino acid residue” is intended to include those amino acid residues which can be phosphorylated by a protein kinase. Phosphorylatable amino acid residues include, but are not limited to, serine, threonine and tyrosine, or phosphorylatable analogs thereof.

As used herein, “target system” is a cell, tissue or organ which is responsible for the genesis or maintenance of a disease state or is responsible for or associated with the condition being treated.

As used herein, biological activity refers to the in vivo activities of a compound or physiological responses that result upon in vivo administration of a compound, composition or other mixture. Biological activity, thus, encompasses therapeutic effects and pharmacokinetic behaviour activity of such compounds, compositions and mixtures. Biological activities can be observed in in vitro systems designed to test such activities.

As used herein, pharmaceutically acceptable derivatives of a conjugate include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The conjugates produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamineand other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl ar heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl ar heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating a cancer.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, EC₅₀ refers to a dosage, concentration or amount of a particular test conjugate that elicits a dose-dependent response at 50% of maximal expression of a particular response that is induced, provoked or potentiated by the particular test conjugate.

It is to be understood that the conjugates provided herein may contain chiral centers. Such chiral centers may be of either the (R) or (S) configuration, or may be a mixture thereof. Thus, the conjugates provided herein may be enantiomerically pure, or be stereoisomeric or diastereomeric mixtures. As such, one of skill in the art will recognize that administration of a conjugate in its (R) form is equivalent, for conjugates that undergo epimerization in vivo, to administration of the conjugate in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound. The instant disclosure is meant to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

As used herein, the nomenclature alkyl, alkoxy, carbonyl, etc. is used as is generally understood by those of skill in this art.

As used herein, alkyl, alkenyl and alkynyl carbon chains, if not specified, contain from 1 to 20 carbons, or 1 to 16 carbons, and are straight or branched. Alkenyl carbon chains of from 2 to 20 carbons, in certain embodiments, contain 1 to 8 double bonds, and the alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, ethene, propene, butene, pentene, acetylene and hexyne. As used herein, lower alkyl, lower alkenyl, and lower alkynyl refer to carbon chains having from about 1 or about 2 carbons up to about 6 carbons. As used herein, “alk(en)(yn)yl” refers to an alkyl group containing at least one double bond and at least one triple bond.

As used herein, “halo”, “halogen” or “halide” refers to F, Cl, Br or I.

As used herein, “carboxy” refers to a divalent radical, —C(O)O—.

As used herein, “alkylene” refers to a straight, branched or cyclic, in certain embodiments straight or branched, divalent aliphatic hydrocarbon group, in one embodiment having from 1 to about 20 carbon atoms, in another embodiment having from 1 to 12 carbons. In a further embodiment alkylene includes lower alkylene. There may be optionally inserted along the alkylene group one or more oxygen, sulfur, including S(═O) and S(═O)₂ groups, or substituted or unsubstituted nitrogen atoms, including —NR— and —N⁺RR— groups, where the nitrogen substituent(s) is(are) alkyl, aryl, aralkyl, heteroaryl, heteroaralkyl or COR′, where R′ is alkyl, aryl, aralkyl, heteroaryl, heteroaralkyl, —OY or —NYY′, where Y and Y′ are each independently hydrogen, alkyl, aryl, heteroaryl, cycloalkyl or heterocyclyl. Alkylene groups include, but are not limited to, methylene (—CH₂—), ethylene (—CH₂CH₂—), propylene (—(CH₂)₃—), methylenedioxy (—O—CH₂—O—) and ethylenedioxy (—O—(CH₂)₂—O—). The term “lower alkylene” refers to alkylene groups having 1 to 6 carbons. In certain embodiments, alkylene groups are lower alkylene, including alkylene of 1 to 3 carbon atoms.

As used herein, the following terms have their accepted meaning in the chemical literature:

| | |
|---|---|
| AcOH | acetic acid |
| CHCl₃ | chloroform |
| conc | concentrated |
| DBU | 1,8-diazabicyclo[5.4.0]undec-7-ene |
| DCM | dichloromethane |
| DME | 1,2-dimethoxyethane |
| DMF | N,N-dimethylformamide |
| DMSO | dimethylsulfoxide |
| DIEA | N-ethyl-N,N-di-isopropylamine |
| EtOAc | ethyl acetate |
| EtOH | ethanol (100%) |
| Et₂O | diethyl ether |
| Hex | hexanes |
| H₂SO₄ | sulfuric acid |
| MeCN | acetonitrile |
| MeOH | methanol |
| Pd/C | palladium on activated carbon |
| TEA | triethylamine |
| THF | tetrahydrofuran |
| TFA | trifluoroacetic acid |

As used herein, the amino acids, which occur in the various amino acid sequences appearing herein, are identified according to their well-known, three-letter or one-letter abbreviations. Other abbreviations, include for example: DS or DSer for D-Serine; TFA for trifluoroacetic acid; Ac for acetyl, Pv for pivaloyl, Bz for benzoyl, Z for CBz and B for Boc.

For the amino acids used in the peptide substrates herein, conservative substitutions can be made or occur such that the substitutions do not eliminate kinase activity. As described herein, substitutions that alter properties of the peptides, such as removal of cleavage sites and other such sites are also contemplated; such substitutions are generally non-conservative, but can be readily effected by those of skill in the art.

Suitable conservative substitutions of amino acids are known to those of skill in this art and can be made generally without altering the biological activity, for example the kinase activity, of the resulting molecule. Exemplary substitutions include, but are not limited to Arginine for Lysine and Serine for Proline.

Other substitutions are also permissible and can be determined empirically or in accord with known conservative substitutions. For example, one or more amino acid residues within the sequence can be substituted by another natural or non-natural amino acid of a similar polarity which acts as a functional equivalent, resulting in a silent alteration. Substitutes for an amino acid within the sequence can be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid.

As used herein, PEG linker represents a polyethylene glycol chain containing the designated number of atoms, other than hydrogen, in the chain between the drug moiety and the substrate, conjugated to the drug moiety at the first end and to the substrate at the second end.

As used herein, alkane linker represents an alkylene group having the designated number of atoms, other than hydrogen, in the chain between the drug moiety and the substrate, conjugated to the drug moiety at the first end and to the substrate at the second end.

The following naming conventions have been used to name the conjugates provided herein:

The conjugates are provided herein are named in four parts: “Drug”-“Point of Attachment and functionality to the “Drug”-“Linker Type (Linker Length)”-“peptide Substrate”. In an exemplary conjugate, the C-terminus of the peptide substrate is attached to the linker moiety.

The drug moieties in exemplary conjugates provided herein have been abbreviated as follows:
Paclitaxel or O¹⁰ deacetyl-paclitaxel=PXL
Vinblastine or O⁴-deacetyl-=VBL
Doxorubicin=DOX

In naming the conjugates, the abbreviated name of the drug is followed by the point of attachment and functionality linking the drug to the C-terminus of the peptide substrate, optinally via linking atoms interdisposed inbetween. The peptide substrate is named by using standard one letter codes for the aminoacids. The amino acids with side chain capping groups are represented by indicating the protecting group in the parenthesis. For example, conjugate Ac-E(Bz1)YIYGSFK(CBz)-PEG(13)-10Ca—PXL is a paclitaxel peptide conjugate, wherein carboxy terminus of the peptide is conjugated to paclitaxel at C10 with a PEG moiety containing 13 atoms in the main chain, other than hydrogen, in the PEG unit, via a carbamate functionality. The peptide substrate contains benzyl capping group on the glutamic acid and CBz group on the lysine side chain. Table 1 provides examples of various drug moieties with possible points of attachments and linking functionalities. Table 2 herein provides examples of various linker groups and the names thereof.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

B. Conjugates

Provided herein are drug-substrate conjugates for use in the methods and compositions for increasing drug efficiency. The drug-substrate conjugates provided herein retain a significant fraction of parent drug activity within the conjugate and the desired therapeutic effect is elicited by the drug-substrate conjugate without having the need to cleave the drug from the substrate.

The conjugates provided herein are not limited to specific drug, linker and substrate moieties. Various combinations of the drug, linker and substrate moieties can be prepared using synthetic methodologies known in the art and described herein. As discussed above, the conjugates can contain a plurality of substrates, a plurality of linkers and a plurality of drug moieties.

In certain embodiments, the drug moiety and/or the substrate moiety in the conjugate can be present in a form of a pharmaceutically acceptable derivative that renders the conjugate biologically inactive. The inactive drug-substrate conjugate can be converted to the active drug-substrate conjugate under physiological conditions without having the need to cleave the drug-substrate conjugate.

In certain embodiments, the conjugates provided herein retain a significant fraction of biological activity within the conjugate. In certain embodiments, the conjugates retain from about 5% up to about 100% of the biological activity, from about 5% up to about 95%, from about 5% up to about 90%, from about 5% up to about 80%, up to about 70%, about 60%, or about 50% of the biological activity. In certain embodiment the biological activity of the drug in the conjugate exceeds that of parent drug. In certain embodiments, the conjugates show improved cytotoxic selectivity than the parent drug. In certain embodiments, the peptide substrates in the conjugates show improved activity than the free peptide substrate.

Without being bound to any theory, in certain embodiments, the drug-substrate conjugates are selectively trapped or accumulated in target cells. In certain embodiments, the substrate is phosphorylated by a kinase whose activity is involved in the condition being treated. As a result, doses of the drug-substrate conjugate required to elicit the same effective amount of therapeutic response as the parent drug can be reduced thereby resulting in a reduction of undesirable side effects. This allows for an increase in the duration of therapy, which is highly desirable in chronic disease settings. In addition, the standard drug dose in conjugate form can be increased without exceeding the tolerability of undesirable side effects to allow for more aggressive treatment. Furthermore, molecules capable of eliciting a desired pharmacological response but which elicit unacceptable side effects at doses below that required for an effective amount of therapeutic response may be transformed by conjugation into a molecule useful in the treatment of a ACAMPS condition. Finally, trapping or accumulation of drug conjugates by phosphorylation may prevent the efflux of cancer drugs such as vinca alkaloids, epipodophyllotoxins, taxanes/taxoids, and anthracyclines, by the membrane transporter P-glycoprotein, thus, preventing a major form of MDR.

In certain embodiments, the substrate moiety in the conjugate may be any substrate for a protein kinase or lipid kinase that is overexpressed, overactive or exhibits undesired activity in a target system. The action of the kinase on the substrate results in a modified conjugate wherein significant fraction of the activity of the drug moiety as well as the substrate moiety is retained. In a target system (e.g. cell, tissue or organ) containing cells, the drug-substrate conjugate is less able to exit the cell in comparison to the unmodified drug. Accumulation of the drug-substratre conjugate into the target cells will occur by pushing the equilibrium of passive diffusion towards the target cells because of preferential trapping or accumulation due to the higher kinase activity in these cell.

In certain embodiments, the drug-substrate conjugates exhibit improved cytotoxic selectivity index over the parent drug. In certain embodiments, the drug-substrate conjugates exhibit improved solubility over the parent drug. In certain embodiments, the conjugates exhibit better serum stability than the parent drug. In certain embodiments, the conjugates exhibit better shelf life than the parent drug.

In one exemplary embodiment, the conjugates for use in the methods and compositions provided herein have the formula (1):
(D)_{d}-(L)_{q}-(S)ₜ (1)
or a pharmaceutically acceptable derivative thereof, wherein D is a drug moiety; d is 1-6, or is 1 or 2; L is a non-releasing linker; q is 0 to 6, or is 0 or 1; S is a substrate for a kinase other than a hexokinase, a protein kinase or a lipid kinase; and t is 1 to 6, or is 1 or 2, or is 1. In the conjugates, the drug moiety is covalently attached, optionally via a non-releasing linker, to the substrate.

In conjugates that contain one or two drug moieties, which are the same or different, conjugated to the substrate moiety(s) or non-releasing linked thereto can be at various positions of the substrate.

In certain embodiments, the conjugates have formula (2):
D-L-S (2)

where the variables are as defined elsewhere herein.

Exemplary substrates, drug moieties, linkers and exemplary conjugates are described in further detail below. It is intended herein that conjugates resulting from all combinations and/or permutations of the groups recited below for the variables of formulae (1) and (2) are encompassed within the instant disclosure.

1. Drug Moiety

The conjugates provided herein are intended for modifying a variety of biological responses. The drug moiety may be any molecule, as well as a binding portion, fragment or derivative thereof that is capable of modulating a biological process. Thus, the drug moiety encompasses any molecule that elicits a pharmacological response that may be used for the treatment or prevention of a disease. Accordingly, the drug moities are any moities, including proteins and polypeptides, small molecules and other molecules that possess or potentiate a desired biological activity. Such molecules include cytotoxic agents, such as, but are not limited to, a toxin such as abrin, ricin A, pseudomonas exotoxin, shiga toxin, diphtheria toxin and other such toxins and toxic portions and/or subunits or chains thereof; proteins such as, but not limited to, tumor necrosis factor, α-interferon, γ-interferon, nerve growth factor, platelet derived growth factor, tissue plasminogen activator; or, biological response modifiers such as, for example, lymphokines, interleukin-I (IL-1), interleukin-2 (IL-2), interleukin-6 (IL-6), granulocyte macrophage colony stimulating factor (GMCSF), granulocyte colony stimulating factor (G-CSF), erythropoietin (EPO), pro-coagulants such as tissue factor and tissue factor variants, pro-apoptotic agents such FAS-ligand, fibroblast growth factors (FGF), nerve growth factor and other growth factors.

The drug moiety of the drug conjugate may be derived from a naturally occurring or synthetic compound that may be obtained from a wide variety of sources, including libraries of synthetic or natural compounds. For example, numerous means are available for random and directed synthesis of a wide variety of organic compounds and biomolecules. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc., to produce structural analogs.

As such, the drug moiety may be obtained from a library of naturally occurring or synthetic molecules, including a library of compounds produced through combinatorial means (i.e., a compound diversity combinatorial library). When obtained from such libraries, the drug moiety employed will have demonstrated some desirable activity in an appropriate screening assay for the activity. Combinatorial libraries, as well as methods for the production and screening, are known in the art.

In particular embodiments, the drug moiety is a chemotherapeutic agent. Examples of chemotherapeutic agents include but are not limited to anti-infective agents, antihelminthic, antiprotozoal agents, antimalarial agents, antiamebic agents, antileiscmanial drugs, antitrichomonal agents, antitrypanosomal agents, sulfonamides, antimycobacterial drugs, or antiviral chemotherapeutics. Chemotherapeutic agents may also be antineoplastic agents or cytotoxic drugs, such as alkylating agents, plant alkaloids, antimetabolites, antibiotics, tubulin binding agents and other anticellular proliferative agents.

Other specific drugs of interest include but are not limited to central nervous system depressants or stimulants, respiratory tract drugs, pharmacodynamic agents, such as histamines and antihistamines, cardiovascular drugs, blood or hemopoietic system drugs, gastrointestinal tract drugs, and locally acting drugs including chemotherapeutic agents. Drug compounds of interest from which drug moieties may be derived are also listed in: Goodman & Gilman's, The Pharmacological Basis of Therapeutics (9th Ed) (Goodman, et al., eds.) (McGraw-Hill) (1996); and 1999 Physician's Desk Reference (1998). and Chu, E.; DeVita, V. T. Physicians' Cancer Chemotherapy Drug Manual 2003, Jones and Bartlett Publishers.

Classes of cytotoxic agents for use herein include, for example, the a) anthracycline family of drugs, b) vinca alkaloid drugs, c) mitomycins, d) bleomycins, e) cytotoxic nucleosides, f) pteridine family of drugs, g) diynenes, h) estramustine, i) cyclophosphamide, j) taxanes, k) podophyllotoxins, l) maytansanoids, m) epothilones, and n) combretastatin and analogs.

In certain embodiments, the drug moiety is selected from a) doxorubicin, b) carminomycin, c) daunorubicin, d) aminopterin, e) methotrexate, f) methopterin, g) dichloromethotrexate, h) mitomycin C, i) porfiromycin, j) 5-fluorouracil, k) 6-mercaptopurine, l) cytosine arabinoside, m) podophyllotoxin, n) etoposide, o) etoposide phosphate, p) melphalan, q) vinblastine, r) vincristine, s) leurosidine, t) vindesine, u) estramustine, v) cisplatin, w) cyclophosphamide, x) Taxol®, y) leurositte, z) 4-desacetylvinblastine, aa) epothilone B, bb) taxotere, cc) maytansanol, dd) epothilone A, and ee) combretastatin and analogs. In certain embodiments, the drug is selected from Paclitaxel, Doxorubicin, Vinblastine, Methotrexate and Cisplatin.

Table 1 provides exemplary drug moieties used in the conjugates provided herein. Also indicated are points of attachment of the linker to the drug moieties and the functionality connecting the drug and the linker.

TABLE 1

| Structure of Drug/Drug Functional Group | |
|---|---|
| Fragment | Abbreviation |
| 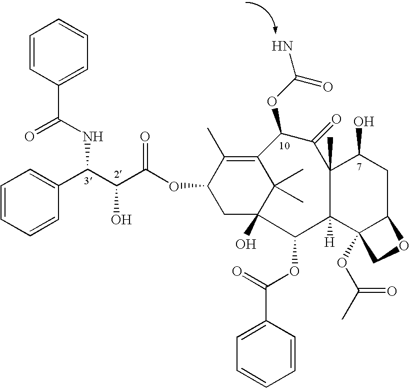 | 10Ca-PXL |
| 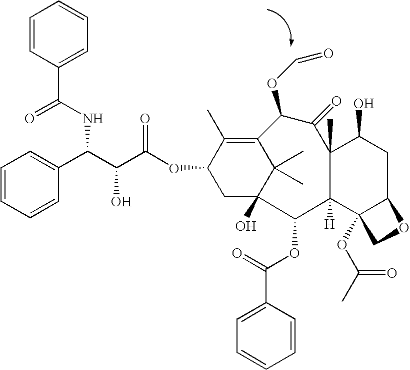 | 10Es-PXL |
| 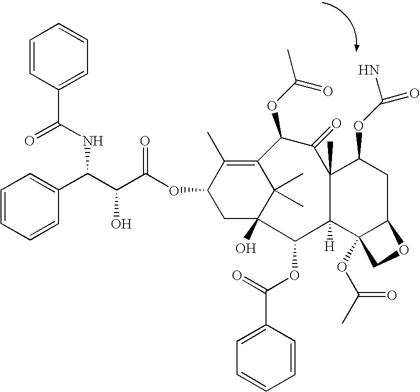 | 7Ca-PXL |
| 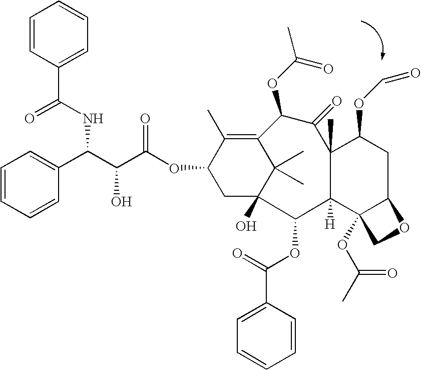 | 7Es-PXL |
| 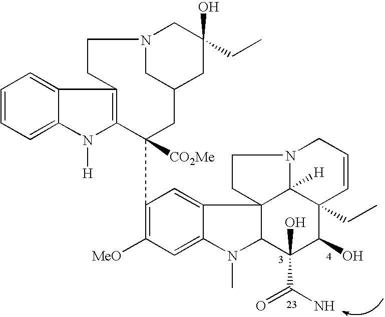 | 3Am-VBL |
| 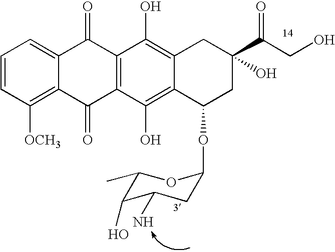 | 3′ALK-DOX |
| 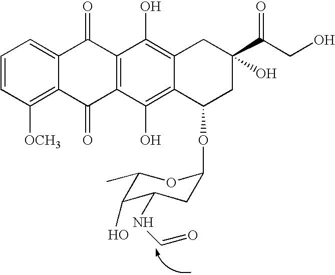 | 3′Am-DOX |

a) Arrows indicates site of attachment to drug (or functionality to drug) from Linker/Spacer fragment of Table X

Furthermore, other drug moieties that may have been tested and considered to have poor properties for treating cancer or proliferative disorders may also be used. When used in the conjugates provided herein, such drug moieties can exhibit enhanced biological activity as compared to the unconjugated drug.

2. Linking Moiety

A linking moiety is used to attach the drug covalently to the substrate. The terms “linker” and “linking moiety” herein refer to any moiety that non-releasably connects the substrate moiety and drug moiety of the conjugate to one another. The linking moiety can be a covalent bond or a chemical functional group that directly connects the drug moiety to the substrate. The linking moiety can contain a series of covalently bonded atoms and their substituents which are collectively referred to as a linking group. Linking moietiess are characterized by a first covalent bond or a chemical functional group that connects the drug moiety to a first end of the linker group and a second covalent bond or chemical functional group that connects the second end of the linker group to the C-terminus of the peptide substrate. The first and second functionality, which independently may or may not be present, and the linker group are collectively referred to as the linker moiety. The linker moiety is defined by the linking group, the first functionality if present and the second functionality if present. As used herein, the linker moiety contains atoms interposed between the drug moiety and substrate, independent of the source of these atoms and the reaction sequence used to synthesize the conjugate.

In one embodiment, the linker moiety is chosen to serve as a spacer between the drug and the substrate, to remove or relieve steric hindrance that may interfere with substrate activity and/or the pharmacological effect of the drug. The linker moiety can also be chosen based on its effect on the hydrophobicity of the drug-substrate conjugate, to improve passive diffusion into the target cells or tissue or to improve pharmacokinetic or pharmacodynamic properties. Thus, linking moieties of interest can vary widely depending on the nature of the drug and substrate moieties. In certain embodiments, the linking moiety is biologically inert. A variety of linking moieties are known to those of skill in the art, which may be used in the conjugates provided herein. Precursors for a variety of linkers are known to those of skill in the art, which may be used in the synthesis of conjugates provided herein. Linker precursors are desirably synthetically accessible and provide shelf-stable products; and do not add any intrinsic biological activity that interferes with the conjugates activity. When incorporated into the conjugates, they can add desirable properties such as increasing solubility or stability to the conjugate.

Any bifunctional linker precursor, in certain embodiments, heterobifunctional linking precursers that can form a non-releasable bond between the drug moiety and the substrate moiety, when incorporated in the conjugate, can be used in the conjugates provided herein. In certain embodiments, the linker precursor can be homobifunctional. In certain embodiments, one or more of substrate moieties are linked to one or more drug moieties via a multifunctional linking moiety.

In one embodiment, a linker precursor has functional groups that are used to interact with and form covalent bonds with functional groups in the components (e.g., drug moiety and substrate moiety) of the conjugates described and used herein. Examples of functional groups on the linker precursor (prior to interaction with other components) include —NH₂, —NHNH₂, —ONH₂, —NHC═(O)NHNH₂, —OH, —CHO, halogen, —CO₂H, and —SH. Each of these functional groups can form a covalent linkage to a suitable functional group on the substrate or the drug to give a drug-linker or substrate-linker construct. For example, amino, hydroxy and hydrazino groups can each form a covalent bond with a reactive carboxyl group (e.g., a carboxylic acid chloride or activated ester such as an N-hydroxysuccinimide ester (NHS)). Other suitable bond forming groups are well-known in the literature.

The linking moiety can include linear or acyclic portions, cyclic portions, aromatic rings or combinations thereof. In certain embodiments, the linking moiety L can have from 1 to 100 main chain atoms other than hydrogen atoms, selected from C, N, O, S, P and Si. In certain embodiments the linking moiety contains up to 50 main chain atoms other than hydrogen, up to 40, up to 30, up to 20, up to 15, up to 10, up to 5, up to 2 main chain atoms other than hydrogen. In certain embodiments the linking moiety is acyclic.

In certain embodiments, the linking moieties contain oligomers of ethylene glycol or alkylene chains or mixtures thereof. These linking moieties are, in certain embodiments, attached to the C-terminus of the substrate via either an alkyl or amide functionality. In certain embodiments, the drug moiety is attached to the first end of the linker via an amide, sulfonamide, or ether functionality and the second end of the linker is attached to the substrate, in certain embodiments, the carboxy terminus of the peptide substrate. Illustrative synthetic schemes for forming such conjugates are discussed elsewhere herein for exemplary linkers for the conjugates provided herein.

In one embodiment, the linking moiety is a covalent bond between the drug moiety and the substrate moiety. This attachment can be accomplished via coupling of a functional group on the drug with a compatible (e.g., linkage-forming) functional group on the substrate. In certain embodiments, the drug has an isocyanate, isothiocyanate or carboxylic acid functional group that is used to attach the drug to a hydroxy or amino group present on the substrate moiety to form a carbamate, thiocarbamate, urea or thiourea linkage between the components.

A variety of linking moieties depending on the nature of the drug and substrate moieties can be used in the conjugates provided herein. Suitable linking moieties can be selected by one of skill in the art based on the criteria set forth herein. In one embodiment, the linking moiety can be selected by the following procedure: A first end of a linker precurser used in synthesizing linker-peptide constructs is subjected to a first test which determines protein kinase activity. The first test may involve observing ADP formation, an obligatory co-product of phospho group transfer from ATP which is catalyzed by the kinase to the hydroxyl group of serine, threonine or tyrosine amino acid in the peptide. Formation of ADP is followed by a coupled enzyme assay. ADP, formed from protein phosphorylation, is used by pyruvate kinase to generate pyruvate from phosphoenolpyruvate which in turn is converted to lactate by lactate dehydrogenase. The lactate results in the consumption of NADH which is followed spectrophotometrically. The rate of peptide phosphorylation is then directly related to the rate of decrease in the observed NADH signal.

Another test may involve monitoring the consumption of ATP. For example, ATP concentrations at time 0 or after 4 hour incubation may be monitored by luciferase reaction (PKLight kit obtained from Cambrex Corporation, One Meadowlands Plaza, East Rutherford, N.J. 07073), which generate a luminescence readout in the presence of ATP. Assays are initiated by mixing a kinase and a peptide in the presence of 40 μM ATP. After 4 hour of incubation at 30° C., PKLight reagent is added and mixed well, and luminescence readout measured. The rate of peptide phosphorylation is then directly related to the rate of decrease in the observed luminescence. Based on the first test, linkers of appropriate lengths and peptides with an effective amount of kinase substrate activity which may be expected to be retained in the drug conjugate may be found.

The linker found in the first test is subjected to a second test in certain embodiments, to determine suitability of the linker by connecting a second end of the linker precursor to a drug moiety. The site on the drug wherein the second end of the linker is attached is known to tolerate modification or may be shown to tolerate modification through a suitable functional group either pre-existing on the drug or on an analog thereof that is known to have an effective amount of the pharmacological activity of the parent drug. Examples of drug analogs known to tolerate modification include but are not limited to paclitaxel modified at C7, C10 and C3′ (Kingston, Fortschr. Chem. Org. Naturst. (2002) 84:53-225); camptothecin analogs with suitable functionalities for linker attachment (Wall, et al., J. Med. Chem. (1993) 36:2689-2700); and vinblastine derivatives prepared from the natural product O⁴-deacetyl vinblastine or from O⁴-deacetyl-3-de-(methoxycarbonyl)-vinblastin-3-yl carbonyl azide through condensation with amines (Lavielle, et al., J. Med. Chem. (1991) 34:1998-2003), or other vindesine derivatives (Barnett, et al., J. Med. Chem. (1978) 21:88-96). Vindesine and O¹⁷-deacetyl-vinblastine are characterized by a free hydroxyl group at C-4.

Drug-linker constructs may further be screened using functional assays predictive of pharmacological activity. In one example, tubulin stabilization for paclitaxel drug linker constructs or tubulin disruption by viblastine drug-linker constructs is determined with a tubulin polymerization assay (Barron, et al., Anal. Biochem. (2003) 315:49-56). Tubulin assembly or inhibition may be monitored by light scattering which is approximated by the apparent absorption at 350 nm. A commercial kit available from Cytoskeleton (Denver, Colo.) may be used for the tubulin polymerization assay. In another example, a functional assay for camptothecin drug-linker constructs depends on inhibition of Topoisomerase I binding to DNA (Demarquay, Anti-Cancer Drugs (2001) 12:9-19).

One skilled in the art will appreciate that the functional assays described here may also be used to screen for direct peptide-drug conjugates (i.e., conjugates which contain no linker). One skilled in the art will also appreciate that appropriate linkers may be found by interchanging the order of the first and second tests described above.

In certain embodiments, the drug and the sphingosine moiety or its analog (alternatively refered to as sphigoids) can be attached through functionalities including, but not limited to, ether, amide, carbamate, urea, ester or alkylamine linkage. For example, if the drug functionality is OH, either on the drug itself or through a spacer, then attachment of a sphingosine moiety or its analog may be made through ether or ester. If the functionality on the sphingosine moiety or its analog is a maleimide and the functionality of the drug is thiol, a Michael addition will take place and the two will be linked through thioether. With a free amine on sphingosine and carboxylic acid on the drug or vice versa, the two components can be linked through amide bond. Where CHO is the functional group on the drug, the amine on the sphingosine may be attached to the drug by reductive amination using NaBH₄, NaCNBH₃, NaB(OAc)₃H or other suitable reducing agents.

Modification or activation of the functionality on the drug, drug spacer or sphingosine or its analogs may be necessary for certain attachment methods. Example A, to obtain a carbamate or urea linkage from a OH or NHR functionality of the drug, drug construct may be treated with carbonyl di-imidazole, phosgene or other carbonyl synthon equivalent. The intermediate may then be subsequently treated with an amine from the sphingosine moiety or its analog. Example B, an OH group on the sphingosine moiety or its analog need to be activated by formation of alkylsulfonates or arylsulfonates before an NHR drug functionality can displace the OH and form a alkylamine linkage.

It is contemplated that drug-linker-sphingosine conjugates have a bulky drug moiety at the end of the lipophilic chain, similar to known pyrene- and NBD-labeled sphingosine derivatives. It is further contemplated that the bulky pyrene moiety will be well tolerated by the kinase, resulting in retention of substrate activity. It is further contemplated that the drug-linker-sphingosine conjugates will exhibit good permeability, based on demonstration that pyrene or NBD-labeled sphingosine can be rapidly incorporated into endothelial or CHO cells.

In one embodiment, the linking moiety in the conjugates provided herein is an alkylene chain containing from 1 up to 50 main chain atoms other than hydrogen. In certain embodiments, the alkylene chain contain 2, 3, 4, 5, 6, 7, 8, 9, 10, 11 or 12 main chain atoms, other than hydrogen. In other embodiments, the alkylene chain contain 3, 4, 5, 6, 7, 8, 9 or 10 main chain atoms, other than hydrogen.

In other embodiment, the linking moiety in the conjugates provided herein contains a polyethylene glycol (PEG) chain. The PEGs for use herein can contain up to 50 main chain atoms, other than hydrogen. In certain embodiments, the PEGs contain 5, 11, 13, 14, 22 or 29 main chain atoms, other than hydrogen. In certain embodiments, the PEGs contain 5, 11, 13 or 29 main chain atoms, other than hydrogen. In other embodiment, the linker moiety contains a combination of alkylene, PEG and maleimide units in the chain.

Some exemplary linking groups incorporated into the conjuagates are provided in Table 2. As exemplified in Table 2, the linking groups are named based on the chemical units present and the number of main atoms, other than hydrogen are indicated in the parenthesis.

TABLE 2

| Structure of Linker Groups | Abbreviation |
|---|---|
| 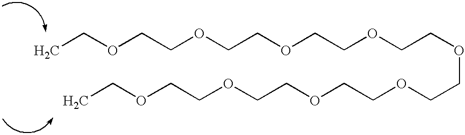 | PEG(29) |
| 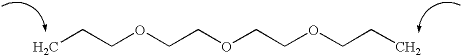 | PEG(13) |
| 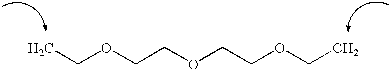 | PEG(11) |
| 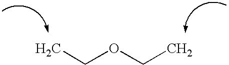 | PEG(5) |
| 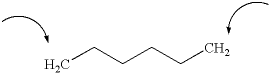 | ALK(6) |
| 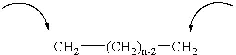 | ALK(n) |
| 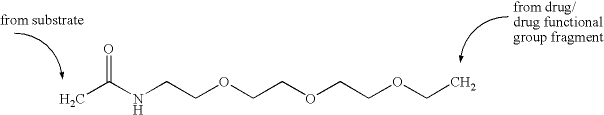 | PEGa(14) |
| 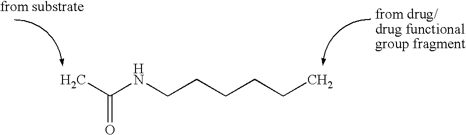 | ALKa(9) |
| 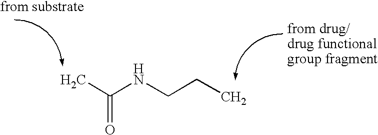 | ALKa(6) |
| 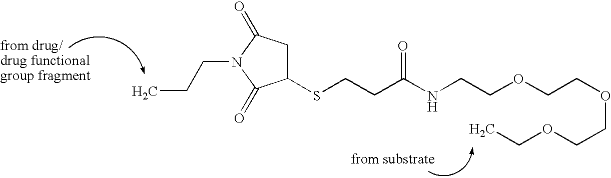 | [MALaPEG[(22) |
| 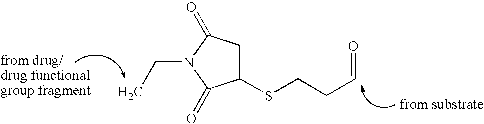 | MAL(8) |
| 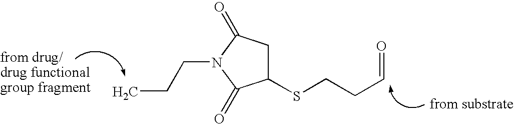 | MAL(9) |

a) Arrows indicates site of attachment to drug (or functionality to drug) and to substrate (or functionality to substrate). For unsymmetrical linker groups directionality of attachment to drug and substrate is so indicated

Several linker precursers useful in the conjugates provide herein are described in U.S. Pat. Nos. 5,512,667; 5,451,463; and 5,141,813. In addition, U.S. Pat. Nos. 5,696,251; 5,585,422; and 6,031,091 describe certain tetrafunctional linking groups that can be used for the conjugates provided herein.

3. Substrates

The substrate moiety may be any substrate for a kinase that is overexpressed, overactive or exhibits undesired activity in a target system, wherein the kinase is a protein kinase or a lipid kinase. The kinase is present at a higher concentration or operates at a higher activity, or the activity is undesired or persistent in a cell type that contributes to the genesis or maintenance of the condition being treated in the target cell in comparison to other cells. Addition of a phosphate group by action of the kinase on the substrate confers a negative charge to the conjugate, thus trapping or accumulating the conjugate inside the targeted cells at concentrations higher than will be achieved in other cells not involved with the condition being treated.

The action of the kinase on the substrate results in a modified conjugate in the target system (e.g. cell, tissue, organ), which is less able to exit the target system in comparison to the unmodified conjugate. In another embodiment, the kinase is associated with an ACAMPS-related condition. In one instance, the action of the protein or lipid kinase on the substrate results in a negative charge on the conjugate.

i. Substrates for Protein Kinase

The substrate for protein kinase is any substrate for a protein kinase that is overexpressed, overactive or exhibits undesired activity in a target system. In one embodiment, the substrate is a peptide for tyrosine and/or serine/threonine kinases known or found to be activated in cells associated with ACAMPS-related conditions. The kinase is present at a higher concentration or operates at a higher activity, or the activity is undesired or persistent in a cell type that contributes to the genesis or maintenance of the condition being treated in comparison to other cells. Addition of a phosphate group by action of the kinase on the peptide confers a negative charge to the conjugate, thus trapping or accumulating the conjugate inside the targeted cells at concentrations higher than will be achieved in other cells not involved with the condition being treated.

Examples of kinases include, but are not limited to, AFK, Akt, AMP—PK, Aurora kinase, beta-ARK, Abl, ATM, ATR, CAK, Cam-II, Cam-III, CCD, Cdc2, Cdc28-dep, CDK, Flt, Fms, Hck, CKI, CKII, Met, DnaK, DNA-PK, Ds-DNA, EGF—R, ERA, ERK, ERT, FAK, FES, FGR, FGF—R, Fyn, Gag-fps, GRK, GRK2, GRK5, GSK, H4-PK-1, IGF—R, IKK, INS—R, JAK, KDR, Kit, Lck, MAPK, MAPKKK, MAPKAP2, MEK, MEK, MFPK, MHCK, MLCK, p135tyk2, p37, p38, p70S6, p74Raf-1, PDGF—R, PD, PhK, PI3K, PKA, PKC, PKG, Raf, PhK, RS, SAPK, Src, Tie-2, m-TOR, TrkA, VEGF—R, YES, or ZAP-70. In some embodiments, the kinase is Akt, Abl, CAK, Cdc2, Fms, Met, EGF—R, ERK1, ERK2, FAK, Fyn, IGF—R, Lck, p70S6, PDGF—R, PI3K, PKA, PKC, Raf, Src, Tie-2 or VEGF—R. In certain embodiments, the kinase is Akt, Src, Tie-2 or VEGF—R. In one example, the kinase is VEGF—R2 (KDR).

In certain embodiments, the peptide substrate for protein kinase contains between 3 to 25 amino acid residues, in other embodiment, 3 to 20 amino acid residues. In certain embodiment, the peptide substrate for protein kinase has formula:
(Xaa)ₙ₁-Zaa-(Xaa)ₘ₁

wherein Zaa is a non-degenerate phosphorylatable amino acid selected from the group consisting of Ser, Thr and Tyr, Xaa is any amino acid and n1 and m1 are integers from 1-10 inclusive.

In other embodiment, certain amino acids can be omitted from the degenerate positions of the peptides of the library such that Zaa is the only phosphorylatable amino acid in the peptides. Accordingly, in another embodiment, when Zaa is Ser or Thr, Xaa is any amino acid except Ser or Thr. In another embodiment, when Zaa is Tyr, Xaa is any amino acid except Tyr. Additionally, non-degenerate amino acid residues can be added to the N-terminal and/or C-terminal ends of the peptides.

In certain embodiments, the phosphorylatable amino acid residue at the fixed non-degenerate position is the only phosphorylatable amino acid residue in the non-phosphorylated peptide.

In certain embodiment, where the protein kinase is a protein-serine/threonine specific kinase, the peptide substrates have Zaa that is a non-degenerate phosphorylatable amino acid selected from Ser and Thr and Xaa is any amino acid except Ser and Thr.

In other embodiment, where the protein kinase is a protein-tyrosine specific kinase, the peptide substrate has Zaa that is Tyr and Xaa is any amino acid except Tyr.

In certain embodiment, where the protein kinase is a dual-specificity kinase, a protein-serine/threonine specific kinase or a protein-tyrosine specific kinase, the peptide substrate contains Zaa that is a non-degenerate phosphorylatable amino acid selected from Ser, Thr and Tyr, and Xaa is any amino acid except Ser, Thr and Tyr.

Another embodiment, the peptide substrate allows for the addition of non-degenerate amino acids at the N-terminal and/or C-terminal ends of the degenerate region of the peptides.

Tables 1A and 1B show a list of kinase substrates for use in the conjugates provided herein. Peptide libraries known in the art may also be used to screen for other peptide substrates for kinases associated with ACAMPS-related conditions. Examples of peptide libraries are described in U.S. Pat. Nos. 5,532,167 and 6,004,757, the disclosures of which are incorporated by reference.

TABLE 1A

| | PEPTIDE |
|---|---|
| KINASE | SEQUENCE |
| Ab1 | EPGPYAQPS |
| Ab1 | TGDTYTAHA |
| AFK | SFTTTAERE |
| AFK | YSFTTTAER |
| Akt-1 | GRPRTSSFAEG |
| Akt-1 | RPRTSSF |
| AMP-PK | FRRLSISTE |
| AMP-PK | EFLRTSAGS |
| AMP-PK | RSSMSGLHL |
| AMP-PK | NRSASEPSL |
| AMP-PK | RRSVSEAAL |
| AMP-PK | LNRMSFASN |
| AMP-PK | RLSISTESQ |
| AMP-PK | QRSTSTPNV |
| AMP-PK | VHNRSKINL |
| AMP-PK | SRTLSVSSL |
| AMP-PK | THVASVSDV |
| AMP-PK | LNRMSFASN |
| autophosphorylation-dependent | ESRISLPLP |
| autophosphorylation-dependent | VTRSSAVRL |
| autophosphorylation-dependent | SRPSSNRSY |
| autophosphorylation-dependent | VRLRSSVPG |
| beta-ARK | MGEASGAQL |
| beta-ARK | QEKESERLA |
| beta-ARK | DPPGTESFV |
| beta-ARK | PGTESFVNA |
| beta-ARK | RNASTNDSP |
| beta-ARK | LSLDSQGRN |
| beta-ARK | STNDSPL |
| branched | SAYRSVDEV |
| branched | IGHHSTSDD |
| CAK | VRTFTHEVV |
| CAK | QMALTPVVV |
| calcium-dependent | TKSASFLKG |
| CaM-II | RRAVSEQDA |
| CaM-II | IGSVSEDNS |
| CaM-II | GRLSSMAMI |
| CaM-II | IRQASQAGP |
| CaM-II | RRAVSELDA |
| CaM-II | GRKASGSSP |
| CaM-II | RRASTIEMP |
| CaM-II | RRQHSYDTF |
| CaM-II | HRQETVEAL |
| CaM-II | HRQETVDAL |
| CaM-II | GRRQSLIQD |
| CaM-II | ARVFSVLRE |
| CaM-II | LLQDSVDFS |
| CaM-II | TRRISQTSQ |
| CaM-II | TRQASQAGP |
| CaM-II | TRTYSLGSA |
| CaM-II | TRQASISGP |
| CaM-II | THYGSLPQK |
| CaM-II | TRQTSVSGQ |
| CaM-II | KYLASASTM |
| CaM-III | ETRFTDTRK |
| CaM-III | RAGETRFTD |
| CaM-III | RFTDTRKDE |
| CCD | NFLKTSAGS |
| cdc2 (CDK-1) | GGGTSPVFP |
| cdc2 | NWHMTPPRK |
| cdc2 | GRPITPPRN |
| cdc2 | AQAASPAKG |
| cdc2 | EFPLSPPKK |
| cdc2 | PGGSTPVSS |
| cdc2 | RLSPSPTSQ |
| cdc2 | STPLSPTRI |
| cdc2 | TTRVTPLRT |
| cdc2 | PLAGSPVIA |
| cdc2 | VPTPSPLGP |
| cdc2 | QTASSPLSP |
| cdc2 | LYSSSPGGA |
| cdc2 | RLRLSPSPT |
| cdc2 | SSVPTPSPL |
| cdc2 | QASSTPLSP |
| cdc2 | QSYSSSQRV |
| cdc2 | KLSPSPSSR |
| cdc2 | SSSSSPSRR |
| cdc2 | TTPLSPTRL |
| cdc2 | GSPRTPRRG |
| cdc2 | DGNKSPAPK |
| cdc2 | DFPLSPPKK |
| cdc2 | FKAFSPKGS |
| cdc2 | IPPHTPVRT |
| cdc2 | NTSSSPQPK |
| cdc2 | DTVTSPQRA |
| cdc2 | SASGTPNKE |
| cdc2 | DLLTSPDVG |
| cdc2 | DKVTSPTKV |
| cdc2 | DTHRTPSRS |
| cdc2 | EGNKSPAPK |
| cdc2 | GGTGTPNKE |
| cdc2 | ENAFSPSRS |
| cdc2 | NVFSSPGGT |
| cdc2 | RQLRSPRRT |
| cdc2 | DAPDTPELL |
| cdc2 | NIYISPLKS |
| cdc2 | EPAVSPLLP |
| cdc2 | SVFSSPSAS |
| cdc2 | WLTKSPDGN |
| cdc2 | PASQTPNKT |
| cdc2 | SPLKSPYKI |
| cdc2 | LKLASPELE |
| cdc2 | SQHSTPPKK |
| cdc2 | PINGSPRTP |
| cdc2 | WLTKTPEGN |
| CDC28-dependent | VIKRSPRKR |
| CDC28-dependent | YTTNSPSKI |
| CDC28-dependent | SVSSSPIKE |
| cdc2-p58cyclin | GSPGTPGSR |
| cdc2-p58cyclin | RPPASPSPQ |
| cdc2-p58cyclin | PSAPSPQPK |
| Cdk5-p23 | PASPSPQRQ |
| Cdk5-p23 | PASPSPQRQ |
| CK | DIPESQMEE |
| CK | YHTTSHPGT |
| CKI | EHVSSSEES |
| CKI | ADSFSLNDA |
| CKI | HDALSGSGN |
| CKI | ESIISQETY |
| CKI | NSVDTSSLS |
| CKI | HVSSSEESI |
| CKI | PLSRTL |
| CKI | ADSFSLHDA |
| CKI | DDAYSDTET |
| CKI | ESLSSSEES |
| CKI | SLSSSEESI |
| CKI | ASATSSSGG |
| CKI | DEEMSETAD |
| CKI | NDALSGSGN |
| CKI | SEENSKKTV |
| CKI | QLSTSEENS |
| CKI | PLSRTLS |
| CKI | QLSTSEENS |
| CKI | SSEESIISQ |
| CKI | VNELSKDIG |
| CKI | WTSDTQGDE |
| CKI | WTSDSAGEE |
| CKI | PPSPSLSRH |
| CKI | LSVSSLPGL |
| CKI | SSEESITRI |
| CKI | LSRHSSPHQ |
| CKII | EQQQTEDEL |
| CKII | DLFGSDDEE |
| CKII | IAADSEAEQ |
| CKII | SEDNSEDEI |
| CKII | NGYISAAEL |
| CKII | EQESSGEED |
| CKII | EDVGSDEED |
| CKII | HSIYSSDDD |
| CKII | SIYSSDDDE |
| CKII | GDRFTDEEV |
| CKII | ENAPSSTSS |
| CKII | EQPGSDDED |
| CKII | ETAESSQAE |
| CKII | AVADSESED |
| CKII | IGSESTEDQ |
| CKII | EDTLSDSDD |
| CKII | ENQASEEED |
| CKII | DEEESEEAK |
| CKII | GSESTEDQA |
| CKII | SGYISSLEY |
| CKII | SEITTKDLK |
| CKII | EQLSTSEEN |
| CKII | SDEESNDDS |
| CKII | MSVEEV |
| CKII | AALESEDED |
| CKII | EEDLSDENI |
| CKII | EESESD |
| CKII | ADSESEDEE |
| CKII | EKEISDDEA |
| CKII | AAVDTSSEI |
| CKII | DLFGSDEED |
| CKII | DKEVSDDEA |
| CKII | FFSSSESGA |
| CKII | MSGDEM |
| CKII | SNDDSDDDD |
| CKII | DYDSSDIED |
| CKII | EENVSVDDT |
| CKII | EDVGSDEEE |
| CKII | SETKTEEEE |
| CKII | GSDVSFNEE |
| CKII | DGNNSDEES |
| CKII | GEINTEDDD |
| CKII | QEGDTDAGL |
| CKII | REQLSTSEE |
| CKII | SPALTGDEA |
| CKII | KGATSDEED |
| CKII | LNDSSEEED |
| CKII | LSDDSFIED |
| CKII | LSGESDLEI |
| CKII | SPHQSEDEE |
| CKII | QLNDSSEEE |
| CKII | REQESSGEE |
| CKII | KMKDTDSEE |
| CKII | LFRLSEHSS |
| CKII | SSSESGAPE |
| CKII | DDEESESD |
| CKII | SSEITTKDL |
| CKII | KKKGSGEDD |
| CKII | KDIGSESTE |
| CKII | KKDASDDLD |
| CKII | TAESSQAEE |
| CKII | TKFASDDEH |
| CKII | TLSDSDDED |
| CKII | PSSTSSSSI |
| CKII | LSEHSSPEE |
| CKII | LELSDDDD |
| CKII | VVELSGESD |
| CKII | VKGATSDEE |
| CKII | LDPLSEPED |
| CKII | TADISEDEE |
| CKII | TSSSSIFDI |
| crystalline | FPFHSPSRL |
| crystalline | STSLSPFYL |
| crystalline | YRLPSNVDQ |
| DnaK | LGGGTFDIS |
| DNA-PK | IDMESQERI |
| ds-DNA | EETQTQDQP |
| ds-DNA | PEETQTQD |
| ds-RNA | LSELSRRRI |
| EGFR | EEQEYIKTV |
| EGFR | EGSAYEEVP |
| EGFR | NPGFYVEAN |
| EGFR | DNPDYQQDF |
| EGFR | HKSGYLSSE |
| EGFR | AEPDYGALY |
| EGFR | FEARYQQPF |
| EGFR | GENIYIRHS |
| EGFR | DADEYLIPQ |
| EGFR | ENAEYLRVA |
| EGFR | EEQEYVQTV |
| EGFR | PVPEYINQS |
| EGFR | QNPVYHNQP |
| EGFR | RLQDYEEKT |
| EGFR | VETTYADFI |
| EGFR | VDEMYREAP |
| endogenous | KNDKSKTWQ |
| ERA | ISITSRKAQ |
| ERA | KISITSRKA |
| ERT | TPPLSPSRR |
| ERT | VEPLTPSGE |
| ERT | TPPLSPIDM |
| ERT | VTPRTPPPS |
| FAK | EEHVYSFPN |
| Fms | LEKKYVRRD |
| Fms | GDSSYKNIH |
| Fms | LEKKYVRRD |
| Fps-gag | VDSAYEVIK |
| GRK | DDSGSAMSG |
| GRK | DDEITQDEN |
| GRK | NDSTSVSAV |
| GRK | NMPSSDDGL |
| GRK | ENTVSTSLG |
| GRK | EKESSNDST |
| GRK | SLDDSGSAM |
| GRK | SNDSTSVSA |
| GRK | EEKESSNDS |
| GRK | SAVASNMRD |
| GRK | NNMPSSDDG |
| GRK | NTVSTSLGH |
| GRK | PVSPSLVQG |
| GRK | QDPVSPSLV |
| GRK | QDENTVSTS |
| GRK | SRKDSLDDS |
| GRK | TVSTSLGHS |
| GRK | STSVSAVAS |
| GRK | RDPVTENAV |
| GRK | SSNDSTSVS |
| GRK2 | DLPGTEDFV |
| GRK2 | GTVPSDNID |
| GRK2 | GRNASTNDS |
| GRK2 | DNIDSQGRN |
| GRK5 | GHQGTVPSD |
| GRK5 | IEQFSTVKG |
| GRK5 | EQFSTVKGV |
| GRK5 | STNDSLL |
| GSK3 | SKIGSTENL |
| GSK3 | NAPVSALGE |
| GSK3 | DEPSTPYHS |
| GSK3 | HHHATPSPP |
| GSK3 | HATPSPPVD |
| GSK3 | RSRASTPPA |
| GSK3 | MPGETPPLS |
| GSK3 | AVVRTPPKS |
| GSK3 | REARSRAST |
| GSK3 | SRSRTPSLP |
| GSK3 | SPQPSRRGS |
| GSK3 | KPGFSPQPS |
| GSK3 | SPSLSRHSS |
| GSK3 | PRPASVPPS |
| GSK3 | SRHSSPHQS |
| GSK3 | SNVSSTGSI |
| GSK3 | TPPKSPSSA |
| GSK3 | REILSRRPS |
| GSK3 | SVPPSPSLS |
| H4-PK-I | VKRISGLIY |
| H4-PK-II | SGRGK |
| haem-controlled | MILLSELSR |
| Hck | EDNEYTARE |
| INSR | GKTDYMGEA |
| INSR | DGNGYISAA |
| INSR | NFDDYMKEV |
| INSR | ELSNYIAMG |
| INSR | EHIPYTHMN |
| INSR | DLSTYASIN |
| INSR | GNGDYMPMS |
| INSR | GSEEYMNMD |
| INSR | FKRSYEEHI |
| INSR | ETDYYRKGG |
| INSR | SRGDYMTMQ |
| INSR | TRDIYETDY |
| INSR | KSLNYIDLD |
| INSR | SSKAYGNGY |
| INSR | YGNGYSSNS |
| INSR | SPGEYVNIE |
| INSR | YETDYYRKG |
| INSR | TDDGYMPMS |
| insulin-sensitive | LDRSSHAQR |
| insulin-sensitive | PLDRSSHAQ |
| isocitrate | GGIRSLNVA |
| Lck/Fyn | MAEAYSEIG |
| Lck/Fyn | QEGLYNELQ |
| MAPK | ELILSPRSK |
| MAPK | GGLTSPGLS |
| MAPK | APVASPAAP |
| MAPK | KVPQTPLHT |
| MAPK | PAAPSPGSS |
| MAPK | SYPLSPLSD |
| MAPK1 (ERK1) | KNIVTPRTP |
| MAPK2 (ERK2) | DLPLSPSAF |
| MAPKAPK2 | SRQLSSGVS |
| MAPKAPK2 | LRGPSWDPF |
| MAPKAPK2 | SRALSRQLS |
| Met | DSDVHVNATY |
| | VNVKCVAP |
| Met | DKEYYSVHN |
| MFPK | GSTSTPAPS |
| MFPK | TRAPSRTAS |
| MHCK | DLKDTKYKL |
| MHCK | ESEKTKTKE |
| MHCK | DEAATKTQT |
| MLCK | ERQKTQTKL |
| MLCK | AEGSSNVFS |
| myosin | AKKMSTYNV |
| myosin | RGRSSVYSA |
| myosin I heavy chain kinase | AGTTYAL |
| p37 | INETSQHHD |
| p37 | VINETSQHH |
| PDGFR | ESVDYVPML |
| PDGFR | GKEIYNTIR |
| PDGFR | RDSNYISKG |
| PhK | NRAITARRQ |
| PhK | GVERSVRPT |
| PhK | GRALSTRAQ |
| PhK | SDEEV |
| PhK | MQLKSEIKQ |
| PhK | LSYRGYSL |
| PhK | SPAISIHEI |
| P13-KINASE | SSNEYMDMK |
| PKA | GRRQSLIED |
| PKA | AVRRSDRAY |
| PKA | ERTNSLPPV |
| PKA | IRRASTIEM |
| PKA | LARRSTTDA |
| PKA | AARLSLTDP |
| PKA | ERRPSNVSQ |
| PKA | RRSSSRPIR |
| PKA | RRRASQLKV |
| PKA | RVRMSADAM |
| PKA | ERRKSHEAE |
| PKA | RRRRSRRAS |
| PKA | DKAKSRPSL |
| PKA | GGRDSRSGS |
| PKA | RRRPTPAML |
| PKA | RRKDYPALH |
| PKA | RRVTSATRR |
| PKA | GRRESLTSF |
| PKA | ARSGSSTYS |
| PKA | HMRSSMSGL |
| PKA | ARKKSSAQL |
| PKA | FRRFTPDSL |
| PKA | EIRVSINEK |
| PKA | SKIGSLDNI |
| PKA | MRRNSFTPL |
| PKA | ERRNSILTE |
| PKA | NTDGSTDYG |
| PKA | FFKKSKIST |
| PKA | DRRVSVAAE |
| PKA | FPRASFGSR |
| PKA | GGRASDYKS |
| PKA | RRKDTPALH |
| PKA | GRTWTLAGT |
| PKA | ARRSTTDAG |
| PKA | ARKFSSARP |
| PKA | FRKLSETES |
| PKA | HTRDSEAQR |
| PKA | ERRLSLVPD |
| PKA | LRRFSLATM |
| PKA | LRRAS |
| PKA | RRRVTSATR |
| PKA | PRRASATSS |
| PKA | RRLSI |
| PKA | ERNLSFEIK |
| PKA | RRRQSVLNL |
| PKA | RRKMSRGLP |
| PKA | RRRLSDSNF |
| PKA | NRQSSQARV |
| PKA | RRKATQVGE |
| PKA | GSRPSESNG |
| PKA | ARNDSVTVA |
| PKA | ERRVSNAGG |
| PKA | HKRKSSQAL |
| PKA | KRXSSQALV |
| PKA | ATRRSYVSS |
| PKA | EIKKSWSRW |
| PKA | SKAGSLGNI |
| PKA | QKRPSQRSK |
| PKA | RRAISGDLT |
| PKA | RVRISADAM |
| PKA | RRRPTPATL |
| PKA | RRKGTDVNV |
| PKA | GRGLSLSRF |
| PKA | GTRLSLARM |
| PKA | RRRGSSIPQ |
| PKA | NRQLSSGVS |
| PKA | RRKASGPPV |
| PKA | RRSSSVGYI |
| PKA | ALRPSTSRS |
| PKA | GSRGSGSSV |
| PKA | TRKISQTAQ |
| PKA | LRRPSDQAV |
| PKA | PRRNSRASL |
| PKA | YRGYSLGNW |
| PKA | SRRSSLGSL |
| PKA | LRGRSFMNN |
| PKA | SRKMSVQEY |
| PKA | KASGSSP |
| PKA | VRFESIRLP |
| PKA | QHLKSVMLQ |
| PKA | SRRLSQETG |
| PKA | QRRSSEGST |
| PKA | SRKESYSVY |
| PKA | VSRTSAVPT |
| PKA | RKFSSARPE |
| PKA | KRRNSEFEI |
| PKA | SSTGSIDMV |
| PKA | KRFGSKAHM |
| PKA | TESQSLTLT |
| PKA | TRKISASEF |
| PKA | LRRLSTKYR |
| PKA | PRRDSTEGF |
| PKA | PSQRSKYLA |
| PKA | WKRTSMKLL |
| PKA | VRRVSDDVR |
| PKA | KRSGSVYEP |
| PKA | SRRQSVLVK |
| PKA | PRRRTRRAS |
| PKA | SRKMSIQEY |
| PKA | VTRRTLSMD |
| PKA | RKRKSSQAL |
| PKA | SRRGSESSE |
| PKA | QRRRSLEPP |
| PKA | SRTPSLPTP |
| PKA | KRKRSRKES |
| PKA | TRRASRPVR |
| PKA | KKSWSRWTL |
| PKA | KREASLDNQ |
| PKA | LRSPSWEPF |
| PKA | TTRRSASKT |
| PKA | LRRFSLATM |
| PKA | TRSVSSSSY |
| PKA | PMRRSVSEA |
| PKA | PRHLSNVSS |
| PKA | PKRGSGKDG |
| PKA | KRRSSSYHV |
| PKA | SPVHSIADE |
| PKA | SRRPSYRKI |
| PKA | PRRRSSFGI |
| PKA | SRKLSDFGQ |
| PKA | SRRDSLFVP |
| PKA | QRRHSLEPP |
| PKA | RHRDTGILD |
| PKA | KRRGSVPIL |
| PKA | KSRPSLPLP |
| PKA | SSRPSSNRS |
| PKA | LRRSSSVGY |
| PKA | LRRASVAQL |
| PKA | PRMPSLSVP |
| PKA | LRKVSKQEE |
| PKA | STSRSLYSS |
| PKA | PKKGSKKAV |
| PKA | SRRPSRATW |
| PKA | KTRSSRAGL |
| PKA | QRRTSLTGS |
| PKA | SRKGSGFGH |
| PKA | SRRASRPVR |
| PKA | QRHGSKYLA |
| PKA | SRTASFSES |
| PKA | KRNSSPPPS |
| PKA | SRKRSGEAT |
| PKA | KLRRSSSVG |
| PKA | KRKNSILNP |
| PKA | TKKTSFVNF |
| PKA | PTRHSRVAE |
| PKA | TPQVSDTMR |
| PKA | KRSNSVDTS |
| PKA | TRKVSLAPQ |
| PKA | LRRPSDQEV |
| PKC | ALGISYGRK |
| PKC | DPTMSKKKK |
| PKC | HRLLTLDPV |
| PKC | AKGGTVKAA |
| PKC | ARKSTRRSI |
| PKC | HKIKSGAEA |
| PKC | SSKRAK |
| PKC | SLKDH |
| PKC | AAASFKAKR |
| PKC | RRADSLQKN |
| PKC | SAYGSVKAY |
| PKC | RVLESFRAA |
| PKC | SFKLSGFSF |
| PKC | DMRQTVAVG |
| PKC | FFRRSKIAV |
| PKC | GSGSSVTSR |
| PKC | EYVQTVKSS |
| PKC | GRVLTLPRS |
| PKC | ERSQSRKDS |
| PKC | AKDASKRGR |
| PKC | DDEASTTVS |
| PKC | KKRFSFKKS |
| PKC | METPSQRRA |
| PKC | LSGFSFKKN |
| PKC | AAASFKAKK |
| PKC | RRRASQLKI |
| PKC | RVRKTKGKY |
| PKC | RQRKSRRTI |
| PKC | SAYATVKAY |
| PKC | SFKKSFKLS |
| PKC | GKSSSYSKQ |
| PKC | DPLLTYRFP |
| PKC | KIQASFRGH |
| PKC | RVRKSKGKY |
| PKC | DEASTTVSK |
| PKC | DRLVSARSV |
| PKC | GRILTLPRS |
| PKC | KSRRTI |
| PKC | GKRQTEREK |
| PKC | GGSVTKKRK |
| PKC | GSGTSSRPS |
| PKC | IDKISRIGF |
| PKC | EGTHSTKRG |
| PKC | NSYGSRRGN |
| PKC | RRRSSKDTS |
| PKC | DSRSSLIRK |
| PKC | SSKRA |
| PKC | FARKSTRRS |
| PKC | GDKKSKKAK |
| PKC | GLGESRKDK |
| PKC | FKRPTLRRV |
| PKC | TKAASEKKT |
| PKC | QGTLSKIFK |
| PKC | LSRFSWGAE |
| PKC | RGRASSHSS |
| PKC | LSGFSFKKN |
| PKC | ASGSFKL |
| PKC | QRVSSYRRT |
| PKC | RVSGSRR |
| PKC | QTVKSSKGG |
| PKC | SPSPSFRWP |
| PKC | KKIDSFASN |
| PKC | TAYGTRRHL |
| PKC | TLASSFKRR |
| PKC | TVTRSYRSV |
| PKC | SSSNTIRRP |
| PKC | TKKQSFKQT |
| PKC | PAAVSEHGD |
| PKC | PFKLSGLSF |
| PKC | YVTTSTRTY |
| PKC | RGKSSSYSK |
| PKC | KTTASTRKV |
| PKC | NRLQTMKEE |
| PKC | YSLGSALRP |
| PKC | RFFGSDRGA |
| PKC | KGQESFKKQ |
| PKC | KKLGSKKPQ |
| PKC | RKAASVIAK |
| PKC | KSRWSGSQQ |
| PKC | LQAISPKQS |
| PKC | KAKVTGRWK |
| PKC | KSKISASRK |
| PKC | SVSSSSYRR |
| PKC | PKDPSQRRR |
| PKC | TRIPSAKKY |
| PKC | LSGLSFKRN |
| PKC | LRMFSFKAP |
| PKC | PSPSSRVTV |
| PKC | VVGGSLRGA |
| PKC | REVSSLKSK |
| PKC | VPTLSTFRT |
| PKC | RAAASRARQ |
| PKC | PSEKSEEIT |
| PKC | RTKRSGSV |
| PKC | STRRSVRGS |
| PKC | TQSTSGRRR |
| PKC | KKRLSVERI |
| PKC | PLSRRLSVA |
| PKC | KISASRKLQ |
| PKC | STLASSFKR |
| PKC | TRGGSLERS |
| PKC | LSGFSFKKS |
| PKC | YTRFSLARQ |
| PKC | VGWPTVRER |
| PKC | NRKPSKDKD |
| PKC | VRKRTLRRL |
| PKC | KRRRSSKDT |
| PKC | QKAQTERKS |
| PKC | VSSSSYRRM |
| PKC | REVSSLKNK |
| PKC | RASSSRSVR |
| PKC | QSRASDKQT |
| PKC | SRGKSSSYS |
| PKC | KKKFSFKKP |
| PKC | GASGSFKL |
| PKC | KKASFKAKK |
| PKC | RTKRSGSV |
| PKC | STRRSIRLP |
| PKC | TVKSSKGGP |
| PKC | KKRFSFKKS |
| PKC | PRRVSRRRR |
| PKC | KIQASFRGH |
| PKC/CAMII | PLSRTLSVS |
| PKC/CAMII | PLRRTLSVA |
| PKG | SARLSAKPA |
| PKG | FRKFTKSER |
| PKG | GPRTTRAQG |
| PKG | RGAISAEVY |
| PKG | LPVPSTHIG |
| PKG | QTYRSFHDL |
| pyruvate | GMGTSVERA |
| pyruvate | DPGVSYRTR |
| pyruvate | YHGHSMSDP |
| Raf | QLIDSMANS |
| Raf-1 | SMANSFVGT |
| RhK | KTETSQVAP |
| RhK | AARGSFDAS |
| RhK | GAFSTVKGV |
| RhK | KTETSQVAP |
| RhK | VGAFSTVKG |
| RhK | TVSKTETSQ |
| RS | RRSRSRSRS |
| RS | PSRRSRSRS |
| RS | SRSRSRSRS |
| RS | SRSRSRSPG |
| RS | SRSRSPGRP |
| S6K | GRASSHSSQ |
| S6K | RRLSSLRAS |
| S6K | RRRLSSLRA |
| sperm-specific | PTKRSPTKR |
| sperm-specific | SPRKSPKKS |
| sperm-specific | PRKGSPRKG |
| sperm-specific | SPKKSPRKA |
| sperm-specific | PTKRSPQKG |
| sperm-specific | PGSPQKR |
| sperm-specific | PRKGSPKRG |
| sperm-specific | KRAASPRKS |
| sperm-specific | SPRKSPRKS |
| sperm-specific | KASASPRRK |
| Src | GIYWHHY |
| Src | YIYGSFK |
| Src | SNPTYSVMR |
| Src | ADDEYAPKQ |
| Src | EEPQYEEIP |
| Src | EEEEYMPME |
| Src | TEDQYSLVE |
| Src | RENEYMPMA |
| Src | PASAYGSVK |
| Src | PPSAYATVK |
| Tie-2 | RLVAYEGWV |
| transforming | ILDTTGQEE |
| tropomyosin | NDMTSL |
| tropomyosin | NDITSL |
| tyk2p135 | SIDEYFSEQ |
| VEGF-R2 (KDR) | QGKDYVGAI |
| VEGF-R2 (KDR) | PEDLYKDFL |
| VEGF-R2 (KDR) | ARDIYKDPD |
| VEGF-R2 (KDR) | KDPDYVRKG |

TABLE 1B

| | |
|---|---|
| KINASE | PEPTIDE SEQUENCE |

| | |
|---|---|
| RGS1_HUMAN | DFRTRESTAKKIK |
| B060-G | PGPQSPGSPLEEE |
| B154-C | GSRSRTPSLPTPP |
| STHM_HUMAN | KELEKRASGQAFE |
| CASB_HUMAN | ALALARETIESLS |
| B257-A | TFPPAPGSPEPPH |
| MPP9_HUMAN | RSPKENLSPGFSH |
| B296-A | PTAGALYSGSEGD |
| OPSD_HUMAN | ASATVSKTETSQV |
| STA4_HUMAN | PSDLLPMSPSVYA |
| A051-D | TPSDSLIYDDGLS |
| KPCE_HUMAN | NNFDQDFTREEPV |
| MPK5_HUMAN | LVNSIAKTYVGTN |
| B176-H | SGAQASSTPLSPT |
| B088-D | AGERRKGTDVNVF |
| MYBB_HUMAN | RKPGLRRSPIKKV |
| A041-B | ASAASFEYTILDP |
| KRAB_HUMAN | APNVHINTIEPVN |
| BLK_HUMAN | ARIIDSEYTAQEG |
| B014-C | RKRSRKESYSVYV |
| B259-A | SDRKGGSYSQAAS |
| TIE1_HUMAN | LSRGEEVYVKKTM |
| TDP2_HUMAN | GFIDQNLSPTKGN |
| LEG3_HUMAN | FSLHDALSGSGNP |
| CGE1_HUMAN | PLPSGLLTPPQSG |
| B170-A | TMTFFKKSKISTY |
| SCG1_HUMAN | ELILKPPSPISEA |
| DCX_HUMAN | STPKSKQSPISTP |
| B204-C | DSQGRNCSTNDSL |
| AAK1_HUMAN | SDGEFLRTSCGSP |
| IF2A_HUMAN | MILLSELSRRRIR |
| RK_HUMAN | AFIAARGSFDGSS |
| MPP5_HUMAN | PPDAADASPVVAA |
| PPBT_HUMAN | TKAQVPDSAGTAT |
| DCX_HUMAN | LLADLTRSLSDNI |
| FX03_HUMAN | QSRPRSCTWPLQR |
| CDK5_HUMAN | GIPVRCYSAEVVT |
| GLK1_HUMAN | EKMWAFMSSRQQT |
| CDK5_HUMAN | LEKIGEGTYGTVF |
| STK9_HUMAN | EGNNANYTEYVAT |
| CFTR_HUMAN | EAILPRISVISTG |
| CIK4_HUMAN | REEEATRSEKKKA |
| G19P_HUMAN | KFEEAERSLKDME |
| RS6_HUMAN | IAKRRRLSSLRAS |
| B054-B | GVRQSRASDKQTL |
| RGS1_HUMAN | SKSKDVLSAAEVM |
| RRPP_HRSVL | DRIDEKLSEILGM |
| EGFR_HUMAN | ISLDNPDYQQDFF |
| MPP5_HUMAN | ESLSYAPSPLQKP |
| GSUB_HUMAN | KKPRRKDTPALHI |
| PLMN_HUMAN | HSWPWQVSLRTRF |
| A051-B | SRKVGPGYLGSGG |
| ITB7_HUMAN | KQDSNPLYKSAIT |
| KAPG_HUMAN | RVKGRTWTLCGTP |
| MIP_HUMAN | KSISERLSVLKGA |
| NMZ1_HUMAN | ITSTLASSFKRRR |
| KG3B_HUMAN | SGRPRTTSFAESC |
| B141-I | SYEEHIPYTHMNG |
| FAK2_HUMAN | RYIEDEDYYKASV |
| IRS1_HUMAN | EETGTEEYMKMDL |
| MBP_HUMAN | TPRTPPPSQGKGR |
| COF1_HUMAN | DMKVRKSSTPEEV |
| B3AT_HUMAN | ATDYHTTSHPGTH |
| B154-F | VDLSKVTSKCGSL |
| B154-I | GAEIVYKSPVVSG |
| MYPC_HUMAN | AGGGRRISDSHED |
| LGN_HUMAN | PKLGRRHSMENME |
| B166-A | FVSNRKPSKDKDK |
| RBL2_HUMAN | DRTSRDSSPVMRS |
| MBP_HUMAN | GLSLSRFSWGAEG |
| G45B_HUMAN | GLVEVASYCEESR |
| CDK7_HUMAN | GSPNRAYTHQVVT |
| RBL2_HUMAN | SKALRISTPLTGV |
| B176-I | DAENRLQTMKEEL |
| KPC2_HUMAN | PPSEGEESTVRFA |
| ERB4_HUMAN | QALDNPEYHNASN |
| LGN_HUMAN | DLLSRFQSNRMDD |
| RRPP_HRSVL | FDNNEEESSYSYE |
| TLE2_HUMAN | EPPSPATTPCGKV |
| CPB6_HUMAN | WKVLRRFSVTTMR |
| EPA3_HUMAN | KLPGLRTYVDPHT |
| WEE1_HUMAN | EEGFGSSSPVKSP |
| EPA2_HUMAN | ESIKMQQYTEHFM |
| KU86_HUMAN | REEAIKFSEEQRF |
| MBP_HUMAN | PLPSHARSQPGLC |
| MPP9_HUMAN | LLSKNESSPIRFD |
| B154-J | PVVSGDTSPRHLS |
| B204-B | VPSDNIDSQGRNC |
| B060-C | AHSIHQRSRKRLS |
| B154-K | DTSPRHLSNVSST |
| LCK_HUMAN | RLIEDNEYTAREG |
| MBP_HUMAN | QGKGRGLSLSRFS |
| A065-D | CSDSTNEYMDMKP |
| B154-H | RVQSKIGSLDNIT |
| ELK1_HUMAN | ISVDGLSTPVVLS |
| A052-A | ELFDDPSYVNVQN |
| B193-A | SQRQRSTSTPNVH |
| B296-C | YSGSEGDSESGEE |
| RON_HUMAN | SALLGDHYVQLPA |
| B008-D | TVSRASSSRSVRT |
| KSYK_HUMAN | ALRADENYYKAQT |
| PMX1_HUMAN | YLSWGTASPYSAM |
| PRGR_HUMAN | DSSESEESAGPLL |
| TLE1_HUMAN | PRASPAHSPRENG |
| KC2B_HUMAN | MSSSEEVSWISWF |
| STHM_HUMAN | SVPEFPLSPPKKK |
| B193-C | PKINRSASEPSLH |
| UGS2_HUMAN | MLRGRSLSVTSLG |
| RON_HUMAN | YVQLPATYMNLGP |
| FA9_HUMAN | SCKDDINSYECWC |
| EF1B_HUMAN | DDIDLFGSDDEEE |
| EPA1_HUMAN | ESIRMKRYILHFH |
| UGS1_HUMAN | PSLSRHSSPHQSE |
| RBL2_HUMAN | RKSVPTVSKGTVE |
| PMX2_HUMAN | WTASSPYSTVPPY |
| COA1_HUMAN | IPTLNRMSFSSNL |
| CYCH_HUMAN | YEDDDYVSKKSKH |
| B154-P | TPGSRSRTPSLPT |
| MLRM_HUMAN | KKRPQRATSNVFA |
| ACM4_HUMAN | CNATFKKTFRHLL |
| CALD_HUMAN | INEWLTKTPDGNK |
| IRS2_HUMAN | GSCRSDDYMPMSP |
| MPP8_HUMAN | RGRRKKKTPRKAE |
| IBP1_HUMAN | LAKAQETSGEEIS |
| CIK6_HUMAN | ANRERRPSYLPTP |
| CALD_HUMAN | EKGNVFSSPTAAG |
| MGP_HUMAN | ESHESMESYELNP |
| K2CF_HUMAN | GAGFGSRSLYGLG |
| K2C8_HUMAN | SAYGGLTSPGLSY |
| B204-F | DFVGHQGTVPSDN |
| PLM_HUMAN | EEGTFRSSIRRLS |
| RBL2_HUMAN | VRYIKENSPCVTP |
| KPB1_HUMAN | SNVSPAISIHEIG |
| IPP1_HUMAN | QIRRRRPTPATLV |
| K2C8_HUMAN | PRAFSSRSYTSGP |
| KPB1_HUMAN | TGIMQLKSEIKQV |
| EG5_HUMAN | LDIPTGTTPQRKS |
| MYBB_HUMAN | LDSCNSLTPKSTP |
| B091-A | YRDVRFESIRLPG |
| B103-A | ARAAARLSLTDPL |
| IPP2_HUMAN | GDDEDACSDTEAT |
| KPCG_HUMAN | TRAAPALTPPDRL |
| KLTK_HUMAN | RDIYRASYYRRGD |
| UGS1_HUMAN | NRTLSMSSLPGLE |
| STA5_HUMAN | DSLDSRLSPPAGL |
| B164-A | SRLRRRASQLKIT |
| B116-B | TPQTQSTSGRRRR |
| TLE2_HUMAN | EPSGPYESDEDKS |
| DCX_HUMAN | YIYTIDGSRKIGS |
| NEK3_HUMAN | FACTYVGTPYYVP |
| B141-C | SSLGFKRSYEEHI |
| RBL2_HUMAN | SPVMRSSSTLPVP |
| A002-C | VSSDGHEYIYVDP |
| SYN1_HUMAN | AGPTRQASQAGPV |
| INR1_HUMAN | PSSSIDEYFSEQP |
| A002-I | SSNYMAPYDNYVP |
| B353-F | VQGEEKESSNDST |
| MYPC_HUMAN | LSAFRRTSLAGGG |
| STK2_HUMAN | NHCDMASTLIGTP |
| CLCB_HUMAN | DFGFFSSSESGAP |
| EPA2_HUMAN | EDDPEATYTTSGG |
| B060-F | QARPGPQSPGSPL |
| KPBB_HUMAN | AGLTAEVSWKVLE |
| FXO1_HUMAN | TFRPRTSSNASTI |
| ELK1_HUMAN | LSTPVVLSPGPQK |
| MIP_HUMAN | KGAKPDVSNGQPE |
| MPP9_HUMAN | TPVTVAYSPKRSP |
| PHS3_HUMAN | SEKRKQISVRGLA |
| RBL2_HUMAN | CIAGSPLTPRRVT |
| B353-A | VANQDPVSPSLVQ |
| VGR3_HUMAN | DIYKDPDYVRKGS |
| B176-K | NGDDPLLTYRFPP |
| PRP5_HUMAN | QNLNEDVSQEESP |
| B204-D | SQGRNCSTNDSLL |
| B154-M | SNVSSTGSIDMVD |
| A008-B | VSQREAEYEPETV |
| CFTR_HUMAN | WTETKKQSFKQTG |
| B154-L | RHLSNVSSTGSID |
| ACHB_HUMAN | WGRGTDEYFIRKP |
| SCG1_HUMAN | AAGERRKSQEAQV |
| SPIN_HUMAN | EYTKEDGSKRIGM |
| KPB1_HUMAN | QVEFRRLSISAES |
| ODBA_HUMAN | DDSSAYRSVDEVN |
| ELK1_HUMAN | QAPGPALTPSLLP |
| MYBB_HUMAN | TPLHRDKTPLHQK |
| C1R_HUMAN | TEASGYISSLEYP |
| INR1_HUMAN | VFLRCINYVFFPS |
| ATF2_HUMAN | SVIVADQTPTPTR |
| MRP_HUMAN | PFKLSGLSFKRNR |
| TRK3_HUMAN | RNLYSGDYYRIQG |
| CST2_HUMAN | NLNGREFSGRALR |
| GLK1_HUMAN | STSIEYVTQRNCN |
| CIK2_HUMAN | PDLKKSRSASTIS |
| MGP_HUMAN | VVTLCYESHESME |
| RBL2_HUMAN | TLYDRYSSPPAST |
| A062-A | TVTSTDEYLDLSA |
| EPB1_HUMAN | DDTSDPTYTSSLG |
| B3AT_HUMAN | EDPDIPESQMEEP |
| B141-D | KKNGRILTLPRSN |
| P2AB_HUMAN | EPHVTRRTPDYFL |
| STA3_HUMAN | NTIDLPMSPRALD |
| A040-E | YASSNPEYLSASD |
| EPA7_HUMAN | TYIDPETYEDPNR |
| MBP_HUMAN | FKLGGRDSRSGSP |
| LGN_HUMAN | AEKHLEISREVGD |
| B066-B | STTTTRRSCSKTV |
| B176-B | QASSTPLSPTRIT |
| B006-B | VGLLKLASPELER |
| PERI_HUMAN | QRSELDKSSAHSY |
| AFX1_HUMAN | RRAASMDSSSKLL |
| 143Z_HUMAN | FYYEILNSPEKAC |
| KPC2_HUMAN | TRHPPVLTPPDQE |
| EDD1_HUMAN | FGMSRNLYAGDYY |
| ACM1_HUMAN | KIPKRPGSVHRTP |
| RBL2_HUMAN | VPTVSKGTVEGNY |
| TY3H_HUMAN | RFIGRRQSLIEDA |
| JAK1_HUMAN | AIETDKEYYTVKD |
| HS9B_HUMAN | PKIEDVGSDEEDD |
| A045-B | FTATEPQYQPGEN |
| B343-A | AALRQLRSPRRTQ |
| PP65_HCMVT | EEDTDEDSDNEIH |
| IPP2_HUMAN | YRIQEQESSGEED |
| B008-B | ERLKLSPSPSSRV |
| MYBB_HUMAN | NSLTPKSTPVKTL |
| MET_HUMAN | DMYDKEYYSVHNK |
| B088-C | EEQEYVQTVKSSK |
| MACS_HUMAN | KRFSFKKSFKLSG |
| VASP_HUMAN | GAKLRKVSKQEEA |
| RBL2_HUMAN | LPVPQPSSAPPTP |
| CIKA_HUMAN | KWTKRTLSETSSS |
| MYC_HUMAN | KKFELLPTPPLSP |
| VGLN_HUMAN | YEEKKKKTTTIAV |
| TRKB_HUMAN | LQNLAKASPVYLD |
| FER_HUMAN | RQEDGGVYSSSGL |
| B189-A | GQKFARKSTRRSI |
| B006-A | KNSDLLTSPDVGL |
| CCAC_HUMAN | PKRGFLRSASLGR |
| KPC2_HUMAN | PEEKTTNTVSKFD |
| ERF_HUMAN | GEAGGPLTPRRVS |
| SRC_HUMAN | LIEDNEYTARQGA |
| ELK1_HUMAN | IHFWSTLSPIAPR |
| PIP4_HUMAN | EGSFESRYQQPFE |
| RGS1_HUMAN | ELKGTTHSLLDDK |
| A072-C | SSQGVDTYVEMRP |
| CN7A_HUMAN | FESERRGSHPYID |
| CDK2_HUMAN | EKIGEGTYGVVYK |
| B014-B | DGKKRKRSRKESY |
| B006-E | VPEMPGETPPLSP |
| MBP_HUMAN | KGRGLSLSRFSWG |
| B227-A | KDGNGYISAAELR |
| B118-B | PAYSRALSRQLSS |
| FGR1_HUMAN | ALTSNQEYLDLSM |
| KPB1_HUMAN | KEFGVERSVRPTD |
| ELK1_HUMAN | LLPTHTLTPVLLT |
| CAS1_HUMAN | ESSISSSSEEMSL |
| HS27_HUMAN | FSLLRGPSWDPFR |
| DYRA_HUMAN | CQLGQRIYQYIQS |
| CFTR_HUMAN | IHRKTTASTRKVS |
| ELK1_HUMAN | GGPGPERTPGSGS |
| CDK2_HUMAN | GVPVRTYTHEVVT |
| B3AT_HUMAN | TEATATDYHTTSH |
| MBP_HUMAN | PGRSPLPSHARSQ |
| B227-C | FDKDGNGYISAAE |
| B116-A | FGPARNDSVIVAD |
| RYNR_HUMAN | VRRLRRLTAREAA |
| DCX_HUMAN | KDLYLPLSLDDSD |
| DCX_HUMAN | HFDERDKTSRNMR |
| EPA2_HUMAN | QLKPLKTYVDPHT |
| EDG1_HUMAN | AGMEFSRSKSDNS |
| CGE2_HUMAN | VCNGGIMTPPKST |
| MBP_HUMAN | FLPRHRDTGILDS |
| IBP1_HUMAN | GSPESPESTEITE |
| LA_HUMAN | GKKTKFASDDEHD |
| CIN6_HUMAN | SKEKIKQSSSSEC |
| CCAC_HUMAN | ASLGRRASFHLEC |
| B154-Q | KKVAVVRTPPKSP |
| MIR1_HUMAN | ILVSTVKSKRREH |
| B015-A | QKRREILSRRPSY |
| MYCN_HUMAN | TSGEDTLSDSDDE |
| B197-B | PLGPLAGSPVIAA |
| MPP9_HUMAN | QCKPVSVTPQGND |
| MBP_HUMAN | SKYLATASTMDHA |
| CAYP_HUMAN | LDRDGSRSLDADE |
| SYN1_HUMAN | PQATRQTSVSGPA |
| F264_HUMAN | LNVAAVNTHRDRP |
| B176-F | NTWGCGNSLRTAL |
| ERB4_HUMAN | IVAENPEYLSEFS |
| B353-K | SNDSTSVSAVASN |
| EGFR_HUMAN | TFLPVPEYINQSV |
| RBL2_HUMAN | DEICIAGSPLTPR |
| EPB1_HUMAN | GSPGMKIYIDPFT |
| STA1_HUMAN | TDNLLPMSPEEFD |
| RBL2_HUMAN | KGTVEGNYVSLTR |
| CALD_HUMAN | SSPTAAGTPNKET |
| RYNR_HUMAN | KKKTAKISQSAQT |
| G19P_HUMAN | YKPLYIPSNRVND |
| MBP_HUMAN | LCNMYKDSHHPAR |
| MBP_HUMAN | RPSQRHGSKYLAT |
| UGS2_HUMAN | FKYPRPSSVPPSP |
| TEC_HUMAN | RYFLDDQYTSSSG |
| FGR3_HUMAN | DVHNLDYYKKTTN |
| VIME_HUMAN | GVRLLQDSVDFSL |
| RYNR_HUMAN | EQGKRNFSKAMSV |
| P53_HUMAN | PSVEPPLSQETFS |
| B170-B | FMSSRRQSVLVKS |
| MACS_HUMAN | FKKSFKLSGFSFK |
| MPI3_HUMAN | SGLYRSPSMPENL |
| RRPP_HRSVL | NEEESSYSYEEIN |
| B006-C | PGETPPLSPIDME |
| PRPC_HUMAN | VISDGGDSEQFID |
| MYC_HUMAN | LLPTPPLSPSRRS |
| ZA70_HUMAN | ALGADDSYYTARS |
| EGFR_HUMAN | GSVQNPVYHNQPL |
| A009-A | PHLDRLVSARSVS |
| A055-D | DKKGNFNYVEFTR |
| KAP3_HUMAN | NRFTRRASVCAEA |
| KCC1_HUMAN | DPGSVLSTACGTP |
| MBP_HUMAN | VDAQGTLSKIFKL |
| B046-A | LVEPLTPSGEAPN |
| TLE1_HUMAN | DPSSPRASPAHSP |
| B130-A | PGKARKKSSCQLL |
| KG3B_HUMAN | RGEPNVSYICSRY |
| MBP_HUMAN | GRASDYKSAHKGF |
| NUCL_HUMAN | DEEEDDDSEEDEE |
| B037-A | SSNDSRSSLIRKR |
| EZRI_HUMAN | KEVHKSGYLSSER |
| A002-G | DMKGDVKYADIES |
| GRK5_HUMAN | LDIEQFSTVKGVN |
| CDK7_HUMAN | GLAKSFGSPNRAY |
| PGDR_HUMAN | DIMRDSNYISKGS |
| B353-E | KAPRDPVTENCVQ |
| MBP_HUMAN | PWLKPGRSPLPSH |
| B066-C | EFPSRGKSSSYSK |
| RGS1_HUMAN | HLESGMKSSKSKD |
| DCOR_HUMAN | VLKEQTGSDDEDE |
| MPK6_HUMAN | ISGYLVDSVAKTI |
| A009-B | RDMYDKEYYSVHN |
| B154-G | KVTSKCGSLGNIH |
| NEF_HV1H2 | SSVIGWPTVRERM |
| CIC2_HUMAN | VCDCKRNSDVMDC |
| MBP_HUMAN | ARTAHYGSLPQKS |
| MR11_HUMAN | EQQLFYISQPGSS |
| CIK4_HUMAN | NLLKKFRSSTSSS |
| B204-E | LCEDLPGTEDFVG |
| MK14_HUMAN | RHTDDEMTGYVAT |
| B176-J | TQGGGSVTKKRKL |
| B141-A | ENVPLDRSSHCQR |
| ADDB_HUMAN | MEQKKRVTMILQS |
| B353-O | TQDENTVSTSLGH |
| TRKB_HUMAN | RDVYSTDYYRVGG |
| PTN1_HUMAN | RSRVVGGSLRGAQ |
| DCX_HUMAN | GPMRRSKSPADSA |
| GRK6_HUMAN | VLDIEQFSTVKGV |
| CRAB_HUMAN | PSFLRAPSWFDTG |
| B073-B | RKGAGDGSDEEVD |
| DCX_HUMAN | TSSSQLSTPKSKQ |
| RS6_HUMAN | LSSLRASTSKSES |
| B059-B | RGGVKRISGLIYE |
| B257-B | AILRRPTSPVSRE |
| EPA4_HUMAN | LNQGVRTYVDPFT |
| UGS2_HUMAN | QASSPQSSDVEDE |
| NAC1_HUMAN | DQARKAVSMHEVN |
| MYPC_HUMAN | SLLKKRDSFRTPR |
| ETS1_HUMAN | CADVPLLTPSSKE |
| CALD_HUMAN | KTPDGNKSPAPKP |
| B008-A | GGPTTPLSPTRLS |
| B353-H | EKESSNDSTSVSA |
| CIK1_HUMAN | DSDLSRRSSSTMS |
| AP50_HUMAN | SQITSQVTGQIGW |
| B046-E | RELVEPLTPSGEA |
| RGS1_HMAN | EAQKVIYTLMEKD |
| TRKC_HUMAN | LHALGKATPIYLD |
| RBL2_HUMAN | DSPSDGGTPGRMP |
| B195-B | KKLERNLSFEIKK |
| RBL2_HUMAN | SGSSDSRSHQNSP |
| ESR1_HUMAN | LHPPPQLSPFLQP |
| A009-D | YVHVNATYVNVKC |
| CASB_HUMAN | TIESLSSSEESIT |
| ACM5_HUMAN | CNRTFRKTFKMLL |
| CFTR_HUMAN | TASTRKVSLAPQA |
| EF2_HUMAN | RAGETRFTDTRKD |
| MPK5_HUMAN | VSTQLVNSIAKTY |
| B1AR_HUMAN | RAGKRRPSRLVAL |
| MPP9_HUMAN | VAYSPKRSPKENL |
| CFTR_HUMAN | INSIRKFSIVQKT |
| B140-A | LTLWTSDSAGEEC |
| MBP_HUMAN | PQKSHGRTQDENP |
| ADDB_HUMAN | GSPSKSPSKKKKK |
| NUCL_HUMAN | AAAAAPASEDEDD |
| ODPT_HUMAN | TYRYHGHSMSDPG |
| CA34_HUMAN | LKGKRGDSGSPAT |
| B154-D | VVRTPPKSPSSAK |
| FRK_HUMAN | KVDNEDIYESRHE |
| RBL2_HUMAN | RLFVENDSPSDGG |
| TLE2_HUMAN | DQPSEPPSPATTP |
| MYBB_HUMAN | SQKVVVTTPLHRD |
| IPPD_HUMAN | RPNPCAYTPPSLK |
| A008-D | YQAEENTYDEYEN |
| MPP8_HUMAN | AFDLFKLTPEEKN |
| FAK1_HUMAN | RYMEDSTYYKASK |
| ODPA_HUMAN | NRYGMGTSVERAA |
| NUCL_HUMAN | KNAKKEDSDEEED |
| B176-D | QRSRGRASSHSSQ |
| LIPS_HUMAN | IAEPMRRSVSEAA |
| STA3_HUMAN | DPGSAAPYLKTKF |
| B116-C | ERNRAAASRCRQK |
| DYRA_HUMAN | KHDTEMKYYIVHL |
| B353-N | EITQDENTVSTSL |
| B006-D | LSPIDMESQERIK |
| KGPA_HUMAN | THIGPRTTRAQGI |
| DCX_HUMAN | SKQSPISTPTSPG |
| PTN1_HUMAN | LRGAQAASPAKGE |
| F26P_HUMAN | PLASPEPTKKPRI |
| SCG1_HUMAN | KEKMKELSMLSLI |
| Z145_HUMAN | HYTLDFLSPKTFQ |
| B159-B | PRSKGQESFKKQE |
| CFTR_HUMAN | LQARRRQSVLNLM |
| PE15_HUMAN | KDIIRQPSEEEII |
| ACM1_HUMAN | CNKAFRDTFRLLL |
| ADDB_HUMAN | KKKFRTPSFLKKS |
| B043-C | RLSSLRASTSKSE |
| MPP8_HUMAN | LMPVSAQTPKGRR |
| MKK2_HUMAN | QSTKVPQTPLHTS |
| MK12_HUMAN | ADSEMTGYVVTRW |
| TLE3_HUMAN | DSLSRYDSDGDKS |
| EPB4_HUMAN | IGHGTKVYIDPFT |
| AMEX_HUMAN | HPGYINFSYEVLT |
| A012-A | RLDGENIYIRHSN |
| Z145_HUMAN | SFGLSAMSPTKAA |
| IRS2_HUMAN | EPKSPGEYINIDF |
| CIN6_HUMAN | TQNVPKDTMDHVN |
| CASB_HUMAN | LARETIESLSSSE |
| B087-A | LLNKRRGSVPILR |
| MBP_HUMAN | HFFKNIVTPRTPP |
| CCAE_HUMAN | EYLTRDSSILGPH |
| VTNC_HUMAN | NQNSRRPSRATWL |
| KRAF_HUMAN | RPRGQRDSSYYWE |
| TRY1_HUMAN | TASSGADYPDELQ |
| MLR5_HUMAN | LRAQRASSNVFSN |
| TYO3_HUMAN | KIYSGDYYRQGCA |
| NMZ1_HUMAN | AITSTLASSFKRR |
| PGDR_HUMAN | SKDESVDYVPMLD |
| A003-A | SGASTGIYEALEL |
| B228-A | CYEQLNDSSEEED |
| MPP9_HUMAN | TKREIMLTPVTVA |
| FYN_HUMAN | QCKDKEATKLTEE |
| DESP_HUMAN | RSGSRRGSFDATG |
| EPA5_HUMAN | EAIKMGRYTEIFM |
| DCX_HUMAN | RYAQDDFSLDENE |
| B296-E | RGLKRSLSEMEIG |
| PRGR_HUMAN | GPFPGSQTSDTLP |
| NPM_HUMAN | AVEEDAESEDEEE |
| ODPT_HUMAN | SMSDPGVSYRTRE |
| CBL_HUMAN | EGEEDTEYMTPSS |
| HMGY_HUMAN | KEEEEGISQESSE |
| ACHD_HUMAN | YISKAEEYFLLKS |
| A002-K | LDTSSVLYTAVQP |
| B176-G | SSVTVTRSYRSVG |
| MBP_HUMAN | FGYGGRASDYKSA |
| CAS1_HUMAN | EKMESSISSSSEE |
| MPP6_HUMAN | EDENGDITPIKAK |
| SSR5_HUMAN | VLCLRKGSGAKDA |
| KPB1_HUMAN | RLSISAESQSPGT |
| A007-A | FLSEETPYSYPTG |
| B311-C | VPWEDRMSLVNSR |
| HS9B_HUMAN | KEREKEISDDEAE |
| MAD3_HUMAN | DSMKDEEYEQMVK |
| MPK1_HUMAN | LIDSMANSFVGTR |
| NS2A_HUMAN | CMDKYRLSCLEEE |
| IRS1_HUMAN | GRKGSGDYMPMSP |
| PEC1_HUMAN | KKDTETVYSEVRK |
| AMPE_HUMAN | EREGSKRYCIQTK |
| CIC2_HUMAN | LEDIKRLTPRFTL |
| B193-B | VKSRWSGSQQVEQ |
| B163-A | AVRDMRQTVAVGV |
| NR41_HUMAN | KEVVRTDSLKGRR |
| KRAC_HUMAN | KDGATMKTFCGTP |
| NS2A_HUMAN | ICRHVRYSTNNGN |
| VASP_HUMAN | LARRRKATQVGEK |
| SYN1_HUMAN | NYLRRRLSDSNFM |
| KKIT_HUMAN | DIKNDSNYVVKGN |
| B189-C | QAIKMDRYKDNFT |
| B197-A | ISSVPTPSPLGPL |
| RB_HUMAN | VNVIPPHTPVRTV |
| EPB3_HUMAN | DAIKMGRYKESFV |
| PEPA_HUMAN | SSTYQSTSETVSI |
| B204-A | GHQGTVPSDNIDS |
| CIK1_HUMAN | LGQTLKASMRELG |
| A057-B | KDKMAEAYSEIGM |
| CST2_HUMAN | HHVPGHESRGPPP |
| B141-H | SLGFKRSYEEHIP |
| A066-A | QQKIRKYTMRRLL |
| B054-A | GQDGVRQSRASDK |
| MPK2_HUMAN | VSGQLIDSMANSF |
| LGN_HUMAN | CQRHLDISRELND |
| TLE1_HUMAN | VSNEDPSSPRASP |
| GPR6_HUMAN | QSKVPFRSRSPSE |
| ELK1_HUMAN | TLTPVLLTPSSLP |
| B060-A | RGAPPRRSSIRNA |
| MPP9_HUMAN | AALSRMPSPGGRI |
| ODBA_HUMAN | TYRIGHHSTSDDS |
| LECI_HUMAN | FQDIQQLSSEEND |
| IPP2_HUMAN | MKIDEPSTPYHSM |
| F26L_HUMAN | RLQRRRGSSIPQF |
| TRKC_HUMAN | FGMSRDVYSTDYY |
| TRKA_HUMAN | HIIENPQYFSDAC |
| B154-B | SGYSSPGSPGTPG |
| A002-E | RPPSAELYSNALP |
| DCX_HUMAN | SPISTPTSPGSLR |
| RB_HUMAN | IYISPLKSPYKIS |
| PH4H_HUMAN | PGLGRKLSDFGQE |
| VINC_HUMAN | KSFLDSGYRILGA |
| IRS2_HUMAN | GGGGGEFYGYMTM |
| C79A_HUMAN | EYEDENLYEGLNL |
| KPC1_HUMAN | SNFDKEFTRQPVE |
| UGS1_HUMAN | RPASVPPSPSLSR |
| PIP4_HUMAN | IGTAEPDYGALYE |
| B197-C | SSMPGGSTPVSSA |
| CFTR_HUMAN | FGEKRKNSILNPI |
| B154-A | GDRSGYSSPGSPG |
| B353-L | TSVSAVASNMRDD |
| MBP_HUMAN | KGVDAQGTLSKIF |
| DBL_HUMAN | FCKRRVESGEGSD |
| Z145_HUMAN | RGKEGPGTPTRSS |
| NEUM_HUMAN | PPTETGESSQAEE |
| STA6_HUMAN | MGKDGRGYVPATI |
| MBP_HUMAN | YGSLPQKSHGRTQ |
| PSA2_HUMAN | VASVMQEYTQSGG |
| RBL2_HUMAN | GLGRSITSPTTLY |
| A011-B | REDSARVYENVGL |
| KPCA_HUMAN | ENFDKFFTRGQPV |
| A008-C | EYEPETVYEVAGA |
| B2AR_HUMAN | KAYGNGYSSNGNT |
| ERB2_HUMAN | TCSPQPEYVNQPD |
| A008-A | KTPSSPVYQDAVS |
| CN5A_HUMAN | GTPTRKISASEFD |
| ESR1_HUMAN | GGRERLASTNDKG |
| NPT2_HUMAN | AKALGKRTAKYRW |
| B088-A | EYVQTVKSSKGGP |
| COA2_HUMAN | RVPTMRPSMSGLH |
| VASP_HUMAN | EHIERRVSNAGGP |
| CIK5_HUMAN | RGVQRKVSGSRGS |
| RB1A_HUMAN | KSNVKIQSTPVKQ |
| B311-A | AGALASSSKEENR |
| B060-H | DLILNRCSESTKR |
| A002-H | ADIESSNYMAPYD |
| PRGR_HUMAN | EQRMKESSFYSLC |
| A011-A | SKRKGHEYTNIKY |
| KPC2_HUMAN | RAKISQGTKVPEE |
| K2C7_HUMAN | SPVFTSRSAAFSG |
| RB_HUMAN | PINGSPRTPRRGQ |
| KAP2_HUMAN | SRFNRRVSVCAET |
| RK_HUMAN | IQDVGAFSTVKGV |
| A066-D | PTAENPEYLGLDV |
| Z145_HUMAN | DEVPSQDSPGAAE |
| EGFR_HUMAN | RHIVRKRTLRRLL |
| CDK4_HUMAN | YSYQMALTPVVVT |
| MIR1_HUMAN | IVAILVSTVKSKR |
| B073-A | AMNREVSSLKNKL |
| KPBB_HUMAN | SKVKRQSSTPSAP |
| PRGR_HUMAN | LRPDSEASQSPQY |
| B196-A | YDPAKRISGKMAL |
| DCX_HUMAN | STPTSPGSLRKHK |
| IF4E_HUMAN | DTATKSGSTTKNR |
| A006-A | TVDGKEIYNTIRR |
| MGP_HUMAN | LCYESHESMESYE |
| STA1_HUMAN | DGPKGTGYIKTEL |
| IRS1_HUMAN | GEEELSNYICMGG |
| MYBB_HUMAN | DNTPHTPTPFKNA |
| ELK1_HUMAN | RDLELPLSPSLLG |
| PAXI_HUMAN | VGEEEHVYSFPNK |
| B046-B | DSFLQRYSSDPTG |
| EFS_HUMAN | GGTDEGIYDVPLL |
| WEE1_HUMAN | YFLGSSFSPVRCG |
| B015-B | EILSRRPSYRKIL |
| HIS1_HUMAN | ISMISADSHEKRH |
| P53_HUMAN | NVLSPLPSQAMDD |
| MBP_HUMAN | HGSKYLATASTMD |
| TRKB_HUMAN | PVIENPQYFGITN |
| B176-C | ERLRLSPSPTSQR |
| MYC_HUMAN | MPLNVSFTNRNYD |
| TRT1_HUMAN | SDTEEQEYEEEQP |
| A063-A | TLTTNEEYLDLSQ |
| REL_HUMAN | KMQLRRPSDQEVS |
| MK10_HUMAN | TSFMMTPYVVTRY |
| M3K5_HUMAN | ATRGRGSSVGGGS |
| MPP5_HUMAN | SGFQVSETPRQAP |
| CD27_HUMAN | HQRRKYRSNKGES |
| B311-D | DRMSLVNSRCQEA |
| MACS_HUMAN | KKKKKRFSFKKSF |
| EPA1_HUMAN | LDDFDGTYETQGG |
| DNB2_ADE04 | MNMLMERYRVESD |
| KPC1_HUMAN | TRQPVELTPTDKL |
| AFX1_HUMAN | PRSSSNASSVSTR |
| STHM_HUMAN | QAFELILSPRSKE |
| A1AA_HUMAN | YVVAKRESRGLKS |
| B060-D | QRSRKRLSQDAYR |
| PA2Y_HUMAN | LNTSYPLSPLSDF |
| B227-B | MARKMKDTDSEEE |
| STA4_HUMAN | TERGDKGYVPSVF |
| KFMS_HUMAN | NIHLEKKYVRRDS |
| EDD1_HUMAN | LLLSNPAYRLLLA |
| RBL2_HUMAN | ELNKDRTSRDSSP |
| Z145_HUMAN | PGPMVDQSPSVST |
| BCKD_HUMAN | ERSKTVTSFYNQS |
| CCAS_HUMAN | EKKRRKMSKGLPD |
| LGN_HUMAN | ILVKCQGSRLDDQ |
| CD3Z_HUMAN | STATKDTYDALHM |
| TR5H_HUMAN | RKSKRRNSEFEIF |
| B197-D | SGISSVPTPSPLG |
| TDP2_HUMAN | FPVSNTNSPTKIL |
| B008-C | KLSPSPSSRVTVS |
| B219-A | ASARAGETRFTDT |
| A061-A | LLAVSEEYLDLRL |
| A041-A | SEHAQDTYLVLDK |
| CFTR_HUMAN | EPLERRLSLVPDS |
| KPCE_HUMAN | TREEPVLTLVDEA |
| B1AR_HUMAN | HGDRPRASGCLAR |
| MBP_HUMAN | KNIVTPRTPPPSQ |
| CAS1_HUMAN | AEPEKMESSISSS |
| B089-A | APTKRNSSPPPSP |
| ACM5_HUMAN | CYALCNRTFRKTF |
| RBL2_HUMAN | KENSPCVTPVSTA |
| EPB1_HUMAN | SAIKMVQYRDSFL |
| DSC2_HUMAN | YNYEGRGSVAGSV |
| PHS1_HUMAN | QEKRRQISIRGIV |
| NS2A_HUMAN | EFPSLRVSAGFLL |
| TLE1_HUMAN | KDSSHYDSDGDKS |
| F26P_HUMAN | NPLMRRNSVTPLA |
| B314-A | SEETPAISPSKRA |
| B063-A | LEHVTRRTLSMDK |
| UGS2_HUMAN | PSGSQASSPQSSD |
| FES_HUMAN | REEADGVYAASGG |
| A056-A | GPPEPGPYAQPSV |
| NEUM_HUMAN | AATKIQASFRGHI |
| KAPB_HUMAN | EEEDIRVSITEKC |
| FGR4_HUMAN | GVHHIDYYKKTSN |
| CRAB_HUMAN | FPTSTSLSPFYLR |
| CIN6_HUMAN | QIEMKKRSPISTD |
| B311-B | LQKKQLCSFEIYE |
| B060-E | QDAYRRNSVRFLQ |
| NEK2_HUMAN | FAKTFVGTPYYMS |
| ACLY_HUMAN | PAPSRTASFYESM |
| B353-M | NMRDDEITQDENT |
| MPK6_HUMAN | LVDSVAKTIDAGC |
| IRS1_HUMAN | VPSSRGDYMTMQM |
| MK10_HUMAN | AGTSFMMTPYVVT |
| B154-O | SSPGSPGTPGSRS |
| B014-A | APAPKKGSKKAVT |
| TRSR_HUMAN | PLSYTRFSLARQV |
| BAN7_HUMAN | EKRHTRDSEAQRL |
| PPLA_HUMAN | RSAIRRASTIEMP |
| FABH_HUMAN | DSKNFDDYMKSLG |
| Z145_HUMAN | LRTHNGASPYQCT |
| HS27_HUMAN | RALSRQLSSGVSE |
| COA1_HUMAN | LALHIRSSWSGLH |
| CST2_HUMAN | GVVWPQGSRQVPV |
| B070-A | QRRSARLSAKPAP |
| B105-A | ITKALGISYGRKK |
| IBP1_HUMAN | NFHLMAPSEEDHS |
| PHOS_HUMAN | ERVSRKMSIQEYE |
| MK07_HUMAN | AEHQYFMTEYVAT |
| ANX2_HUMAN | HSTPPSAYGSVKA |
| A051-E | TWIENKLYGMSDP |
| MEFA_HUMAN | KGMMPPLSEEEEL |
| MBP_HUMAN | RDTGILDSIGRFF |
| A044-A | NENTEDQYSLVED |
| B025-B | AKAKTRSSRAGLQ |
| IL7R_HUMAN | SLPDHKKTLEHLC |
| A002-F | TGESDGGYMDMSK |
| ABL2_HUMAN | RLMTGDTYTAHAG |
| G19P_HUMAN | SLKDMEESIRNLE |
| CENC_HUMAN | HHKLVLPSNTPNV |
| A007-B | YPTGNHTYQEIAV |
| B088-E | IENEEQEYVQTVK |
| B154-E | NVKSKIGSTENLK |
| A051-A | AQAFPVSYSSSGA |
| EZRI_HUMAN | LMLRLQDYEEKTK |
| PRGR_HUMAN | EVEEEDSSESEES |
| A002-J | YMAPYDNYVPSAP |
| RB_HUMAN | AVIPINGSPRTPR |
| B2AR_HUMAN | ELLCLRRSSLKAY |
| NR41_HUMAN | GRRGRLPSKPKQP |
| RRPP_HRSVL | LHTLVVASAGPTS |
| CIN6_HUMAN | KNGCRRGSSLGQI |
| VGLN_HUMAN | EEKKKKTTTIAVE |
| UGS1_HUMAN | TSGSKRNSVDTAT |
| EPB1_HUMAN | AIKMVQYRDSFLT |
| VGR1_HUMAN | TSMFDDYQGDSST |
| PTK6_HUMAN | ALRERLSSFTSYE |
| PIG2_HUMAN | YDVSRMYVDPSEI |
| IBP1_HUMAN | FHLMAPSEEDHSI |
| PIG2_HUMAN | RDINSLYDVSRMY |
| P53_HUMAN | PPLSQETFSDLWK |
| DCX_HUMAN | DLYLPLSLDDSDS |
| NRF1_HUMAN | DEDSPSSPEDTSY |
| CIK3_HUMAN | EELRKARSNSTLS |
| B091-B | QCALCRRSTTDCG |
| KBF1_HUMAN | FVQLRRKSDLETS |
| UGS1-HUMAN | MPLNRTLSMSSLP |
| B195-A | FMRLRRLSTKYRT |
| B257-C | ECNSSTDSCDSGP |
| B154-N | HQDQEGDTDAGLK |
| PE15_HUMAN | TKLTRIPSAKKYK |
| MPP5_HUMAN | GSGLLCVSPWPFV |
| RBL2_HUMAN | DSRSHQNSPTELN |
| DCX_HUMAN | GIVYAVSSDRFRS |
| B046-J | STAENAEYLRVAP |
| CASB_HUMAN | IESLSSSEESITE |
| IRS2_HUMAN | RSPLSDYMNLDFS |
| MYCN_HUMAN | SGEDTLSDSDDED |
| Q13541 | PPGDYSTTPGGTL |
| ZA70_HUMAN | LGADDSYYTARSA |
| K6B1_HUMAN | RQTPVDSPDDSTL |
| Q9UP94 | DDSIISSLDVTDI |
| MPK1_HUMAN; MPK2_HUMAN | SGQLIDSMANSFV |
| CHK2_HUMAN | ETSLMRTLCGTPT |
| GBT1_HUMAN | FERASEYQLNDSA |
| TF_HUMAN | GQSWKENSPLNVS |
| MPK1_HUMAN; MPK2_HUMAN | IDSMANSFVGTRS |

The peptide substrates for use herein can contain natural and/or non-natural amino acids. In certain embodiments, the substrate is a peptide substrate for Akt, Src, Tie-2 or VEGFR. In certain embodiments, the substrate is for Akt or Src. The drug-peptide conjugate, in one embodiment, is effective in treating cancer through phosphorylation of the conjugate by Akt, Src, Tie-2 or VEGF—R, leading in certain embodiment, to trapping or accumulation of the conjugate and hence the anti-cancer agent within the cancer cell or tumor associated endothelial cell. Therefore, trapping or accumulation is responsible for the therapeutic effect of these conjugates in the treatment of cancer. The therapeutic effect of the drug conjugate is not dependent on release of free drug. Therefore, no further intervention of intracellular proteins is required for activation of the drug within the conjugate.

The substrate is non-releasably conjugated to a drug moiety with or without a linker via its carboxy terminus. The N-terminus of the peptide can be free or suitably capped with a capping group. Exemplary capping groups for the N-terminal amino acids for use herein include, but are not limited to, acetyl, benzoyl, pivaloyl, CBz and BOC.

In certain embodiments, the peptide substrates contain amino acids with reactive groups in the side chains, including but not limited to lysine, aspartic acid, and glutamic acid. The amino acids containing reactive groups in the side chain, such as Lys and Glu, can be optionally capped with side chain capping groups. Such groups include, acetyl, benzoyl, pivaloyl, CBz, BOC, t-butyl and DMAB capping group.

In certain embodiments, the peptide substrates contain at least one amino acid selected from tyrosine, threonine, serine, glycine, glutamic acid, proline and arginine. In certain embodiments, the peptide substrates contain at least one amino acid selected from tyrosine, threonine and serine. In certain embodiments, the peptide substrates contain at least one tyrosine. In certain embodiments, the peptide substrates contain at least one serine. In certain embodiments, the peptide substrates contain at least one threonine.

In certain embodiments, the peptide substrates contain an amino acid sequence wherein the phosphorylation site is capped with a suitable capping group. In such cases, the capping group is removed under physiological conditions before the peptide is phosphorylated. In other embodiments, an amino acid residue adjucent to the site of phosphorylation in the peptide substrate can be masked thereby blocking the action of the kinase. In such cases, removal of the masking group under physiological conditions allow for phosphorylation of the peptide substrate.

In other embodiment, the substrate may be capped by an additional amino acid sequence which blocks or diminishes binding of the conjugate to the kinase. Action of a protease on the additional amino acid sequence can change a recognition site for the protease and will generate a conjugate composed of a more competent kinase substrate.

a). Peptide Substrates for Akt

The serine/threonine kinase Akt signal transduction pathway has been found to be one of the most commonly activated pathway in tumor cells. Akt has been found to be overexpressed or aberrantly activated in almost all tumor types (West, K. A., et al., Drug Resist. Update (2002) 5:234-248 and Chang, F., et al., Leukemia (2003) 17:590-603). For example, Akt RNA and protein is overexpressed in ovarian and breast tumors. The gene is amplified in pancreatic and breast tumors. The phosphatase PTEN, which negatively regulates Akt activity, is deleted or inactivated in gliomas, melanomas, ovarian, prostate, breast and colorectal carcinoma. In addition, PTEN overexpression suppresses malignant transformation. Ras activation and tyrosine kinase overexpression are both associated with elevated Akt activity.

Akt is induced by hypoxia and has been shown to stimulate tumor cell proliferation, protect tumor cells from drug induced apoptosis, promote cell invasion and stimulate angiogenesis (Hill, M. M., and Hemmings, B. A., Pharmacol. Ther. (2002) 93:243 251). Akt inhibition blocks tumor growth and induces apoptosis. Several peptide substrates for Akt have been identified, including several with 5-30 micromolar Kₘs (Alessi, D. R., et al., FEBS Lett (1996) 399:333-338). Two of the peptides (RPRAATF and RPRTSTF) exhibit specificity with respect to related MAP kinase and S6 kinase and contain only two positively charged amino acids.

In certain embodiments, the peptide substrate for Akt contains an amino acid sequence:
Xaa1-Xaa2-Xaa3-Xaa4-Xaa5-Xaa6-Xaa7-Xaa8-Xaa9,

The length of a peptide which can be used as a substrate is variable. In certain embodiments, a peptide as short as 3 amino acids in length may be used as a substrate. Accordingly, Xaa1, Xaa1-Xaa2, Xaa1-Xaa2-Xaa3, Xaa9, Xaa8-Xaa9 and Xaa7-Xaa8-Xaa9 may or may not be present within the substrate. For example, the substrate may only be composed of Xaa2-Xaa3-Xaa4-Xaa5-Xaa6-Xaa7-Xaa8 or Xaa3-Xaa4-Xaa5-Xaa6-Xaa7 or Xaa4-Xaa5-Xaa6.

In certain embodiments, the peptide substrate may be longer than 9 amino acids.

In certain embodiments,

Xaa7 is selected from serine, D-serine and threonine;

Xaa6 is selected from serine, lysine, arginine, tyrosine, glutamic acid and phenylalanine;

Xaa5 is selected from serine, threonine, tyrosine, alanine and lysine;

Xaa4 is arginine;

Xaa3 is any amino acid;

Xaa2 is arginine;

Xaa1 is glycine, arginine, lysine, phenylalanine, proline or serine;

Xaa8 is phenylalanine, arginine, valine ortyrosine; and

Xaa9 is serine, glycine, alanine, proline, threonine, glutamic acid or glutamine.

In certain embodiments, the substrate has formula:
(Xaa0)p-(Xaa1)q-Xaa2-Xaa3-Xaa4-Xaa5-Xaa6-Xaa7-Xaa8-(Xaa9)r-(Xaa10)s-(Xaa11)t

where p,q and r are each independently 0 or 1;

Xaa0 is glycine, arginine, lysine, phenylalanine, proline or serine;

Xaa10 is glutamic acid;

Xaa11 is glycine; and

the other amino acid residues are selected as described elsewhere herein.

In certain embodiments, Xaa7 is serine or D-serine.

In certain embodiments, Xaa6 is selected from serine, lysine, glutamic acid, arginine, tyrosine and phenylalanine. In certain embodiments, Xaa6 is serine or glutamic acid. In certain embodiments, Xaa6 is serine.

In certain embodiments, Xaa5 is selected from serine, threonine, tyrosine, alanine and lysine. In certain embodiments, Xaa5 is threonine or lysine. In certain embodiments, Xaa5 is threonine.

In certain embodiments, Xaa3 is proline or serine.

In certain embodiments, Xaa1 is glycine or arginine.

In certain embodiments, Xaa8 is phenylalanine or tyrosine. In certain embodiments, Xaa8 is phenylalanine.

In certain embodiments, Xaa9 is serine, glycine, alanine, proline, threonine, glutamic acid or glutamine. In certain embodiments, Xaa9 is phenylalanine.

In certain embodiments, Xaa10 is glutamic acid. In certain embodiments, Xaa11 is glycine.

In certain embodiments, the peptide substrates for Akt are selected from:

| | |
|---|---|
| Gly-Arg-Pro-Arg-Thr-Ser-Ser- | (SEQ ID NO. 5) |
| Phe-Ala-Glu-Gly; | |
| Gly-Arg-Pro-Arg-Thr-Ser-DSer- | (SEQ ID NO. 1406) |
| Phe-Ala-Glu-Gly; | |
| Gly-Arg-Pro-Arg-Ala-Ala-Ala-Phe- | (SEQ ID NO. 1407) |
| Ala-Glu-Gly; | |
| Arg-Ser-Arg-Thr-Ser-Ser-Phe-Ala- | (SEQ ID NO. 1408) |
| Glu-Gly; | |
| Gly-Arg-Ser-Arg-Thr-Ser-Ser-Phe- | (SEQ ID NO. 1409) |
| Ala-Glu-Gly; | |
| Arg-Pro-Arg-Thr-Ser-Ser-Phe; | (SEQ ID NO. 6) |
| Arg-Ser-Arg-Thr-Ser-Ser-Phe | (SEQ ID NO. 1410) |
| and | |
| Arg-Pro-Arg-Lys-Glu-Ser-Tyr. | (SEQ ID NO. 1411) |

In certain embodiments, the peptides are conjugated to the drug moiety via the carboxy terminus. The N-terminal amino acids in the peptides can be free or capped with a suitable capping group kown in the art. The capping groups for the N-terminal amino acids for use herein include, but are not limited to, acetyl, benzoyl, pivaloyl, CBz and BOC. The amino acids containing reactive groups in the side chain, such as Lys and Glu, can be optionally capped with side chain capping groups. Such groups include, acetyl, benzoyl, pivaloyl, CBz, BOC, t-butyl, benzyl and DMAB capping group.

b). Peptide Substrates for Src

Expression of the Src (oncogene) protein kinase is elevated and directly associated with the malignant phenotype in a wide variety of tumor types, including breast and colorectal cancer (Frame, M. C., Biochim. Biophys. Acta (2002) 1602:114-130; Biscardi, J. S., et al., Breast Cancer Res. (2000) 2:203-210; Irby, R. B., and Yeatman, T. J., Oncogene (2000) 19:5636-5642, for reviews). Several peptide substrates for Src suitable for use herein have been reported in the literature (Lou, Q., et al., Bioorg. Med. Chem. (1996) 4:677-682, and Alfaro-Lopez, J., et al., J. Med. Chem. (1998) 41:2252-2260).

In the conjugates provided herein, in certain embodiments, the peptide substrate for Src contains an amino acid sequence:
(P1)a-P2-P3-P4-P5-(P6)_{b}-(P7)_{c},

wherein a, b and c are each independently 0 or 1;

P1 is selected from tyrosine, phenylalanine, tryptophan, tyrosine, tryptophan and serine;

P2 is selected from isoleucine, leucine and valine;

P3 is tyrosine or D-tyrosine;

P4 is glycine; serine or alanine;

P5 is serine, threonine, alanine, valine, glycine, tyrosine or lysine;

P6 is phenylalanine, tyrosine, D-phenylalanine, D-tyrosine or N-methylphenylalanine; and

P7 is lysine, arginine, serine, histidine, D-lysine, 2,4-diamino-n-butyric acid (Dab), 2,3-diaminopropionic acid (Dap) or ornithine.

In other embodiments, the peptide substrate for Src contains amino acid sequence where P1 is selected from tyrosine, phenylalanine, tryptophan and tyrosine.

In other embodiments, P1 is tyrosine.

In certain embodiments, the peptide substrate for Src contains amino acid sequence where P2 is selected from isoleucine, leucine and valine.

In certain embodiments, the peptide substrate for Src contains amino acid sequence where P2 is isoleucine.

In certain embodiments, the peptide substrate for Src contains amino acid sequence where P3 is tyrosine.

In certain embodiments, the peptide substrate for Src contains amino acid sequence where P4 is glycine.

In certain embodiments, the peptide substrate for Src contains amino acid sequence where P5 is serine, threonine or alanine.

In certain embodiments, the peptide substrate for Src contains amino acid sequence where P5 is serine.

In certain embodiments, the peptide substrate for Src contains amino acid sequence where P6 is phenylalanine or tyrosine.

In certain embodiments, the peptide substrate for Src contains amino acid sequence where P7 is lysine, Dab, Dap or ornithine.

In certain embodiments, the peptide substrate for Src contains amino acid sequence where P7 is lysine.

In certain embodiments,

P2 is selected from isoleucine, leucine and valine;

P3 is tyrosine;

P4 is Glycine; and

P5 is serine, threonine or alanine and other amino acids are selected as defined elsewhere herein.

In certain embodiments, the peptide substrate for Src contains amino acid sequence where

P2 is selected from isoleucine, leucine and valine; and

P5 is serine, threonine or alanine and other amino acids are selected as defined elsewhere herein.

In certain embodiments, the peptide substrate for Src contains amino acid sequence where P2 is isoleucine, P3 is tyrosine, P4 is glycine and P5 is serine.

In certain embodiments, the peptide substrate for Src contains amino acid sequence where P3 is tyrosine, and P4 is glycine.

In certain embodiments, the peptide substrate for Src contains an amino acid sequence:
(P0)ₐ₁(P1)ₐ-P2-P3-P4-P5-(P6)_{b}-(P7)_{c},

where a1 is 0 or 1 and P0 is glutamic acid.

Exemplary peptide substrates for use in the conjugates provided herein are selected from:

| | |
|---|---|
| Tyr-Ile-Tyr-Gly-Ser-Phe-Lys; | (SEQ ID NO. 668) |
| Glu-Tyr-Ile-Tyr-Gly-Ser-Phe-Lys: | (SEQ ID NO. 1412) |
| Tyr-Ile-Tyr-Gly-Ser-Phe-Arg; | (SEQ ID NO. 1413) |
| Tyr-Ile-DTyr-Gly-Ser-Phe-Arg; | (SEQ ID NO. 1414) |
| Tyr-Ile-Phe-Gly-Ser-Phe-Arg | (SEQ ID NO. 1415) |
| Glu-Tyr-Ile-Tyr-Gly-Ser-Phe-Lys; | (SEQ ID NO. 1416) |
| Glu-Tyr-Ile-Tyr-Gly-Ser-Phe-Arg; | (SEQ ID NO. 1417) |
| Tyr-Ile-Tyr-Gly-Ser-Phe-Ser; | (SEQ ID NO. 1418) |
| Tyr-Ile-Tyr-Gly-Ser-Phe-His; | (SEQ ID NO. 1419) |
| and | |
| Gly-Ile-Lys-Trp-His-His-Tyr. | (SEQ ID NO. 1420) |

In certain embodiments, the peptide substrate for Src is selected from:

| | |
|---|---|
| Tyr-Ile-Tyr-Gly-Ser-Phe-Arg; | (SEQ ID NO. 1413) |
| and | |
| Glu-Tyr-Ile-Tyr-Gly-Ser-Phe-Lys. | (SEQ ID NO. 1412) |

In certain embodiments, the peptides are conjugated to the drug moiety via the carboxy terminus. The N-terminal amino acids in the peptides can be free or capped with a suitable capping group kown in the art. The capping groups for the N-terminal amino acids for use herein include, but are not limited to, acetyl, benzoyl, pivaloyl, CBz and BOC. The amino acids containing reactive groups in the side chain, such as Lys and Glu, can be optionally capped with side chain capping groups. Such groups include, acetyl, benzoyl, pivaloyl, CBz, BOC, t-butyl, benzyl and DMAB capping group.

c). Peptide Substrates for Tie-2

Tie-2 is an endothelial cell-specific receptor tyrosine kinase that has been shown to be essential for angiogenesis. This enzyme is over-expressed in tumor vasculature and has been reported to be induced by hypoxia. Tie-2 ligands contain a family of proteins called angiopoietins. Inhibition of Tie-2 signaling results in suppression of tumor angiogenesis and tumor growth. In one embodiment, the peptide for Tie-2 for use in the conjugates provided herein contains a nine amino acid sequence with one positive and one negative charge (Arg-Leu-Val-Ala-Tyr-Glu-Gly-Trp-Val SEQ ID NO. 1421). This peptide shows a relatively high affinity substrate for Tie-2 (Kₘ 119 micromolar) (Deng, S. J., et al., Comb. Chem. High Throughput Screen (2001) 4:525-533). The N-terminus of the peptide can be free or capped with a suitable capping group, in certain embodiments, a pivaloyl group. The side chain of glutamic acid can be free or capped with benzyl group.

d). Peptide Substrates for Met kinase

Met kinase is the high affinity receptor for hepatocyte growth factor. This kinase is overexpressed in several tumor types, including melanoma, glioma, hepatoma, breast, pancreatic and colon carcinomas. Overexpression of Met in gliomas protects from apoptosis. Inhibition of Met sensitizes colorectal tumor cells to apoptosis and blocks breast carcinoma tumorigenesis and metastasis (van der Voort, R., et al., Adv. Cancer Res. (2000) 79:39-90, for review). Hypoxia has been shown to induce Met expression (Pennacchietti, S., et al., Cancer Cell (2003) 3:347-361). In one embodiment, the peptide for use in the conjugates provided herein contains 18 amino acid sequences with a Kₘ of 67 micromolar (two negative charges and one positive charge), (DSDVHVNATYVNVKCVAP). (Hays, J. L., and Watowich, S. J., J. Biol. Chem. (2003) 278:27456-27463).

ii. Substrates for Lipid Kinases

In other embodiments, the substrate is a substrate for lipid kinase, including, but not limited to, sphingosine kinase, phosphoinositol kinase and diacylglycerol kinase. In another embodiment, the substrate is contemplated to be a substrate for sphingosine kinase, such as sphingosine or derivatives thereof. Sphingosine, a molecule condensed from palmitoyl CoA and serine, is one of the sphingolipid metabolites (ceramide, sphingosine, and sphingosine-1-phosphate) playing an important role in the regulation of cell proliferation, survival, and cell death. Sphingosine is biologically produced from ceramide by hydrolysis of the N-acyl group, and sphingosine-1-phosphate is generated from sphingosine by phosphorylation. Ceramide and sphingosine inhibit proliferation and promote apoptosis, while sphingosine-1-phosphate (S1P) stimulates growth and suppresses ceramide-mediated apoptosis. It is generally believed that the balance between the levels of ceramide, sphingosine and sphingosine-1-phosphate represents an important factor in cell fate determination. Sphingosine kinase, the enzyme that phosphorylates sphingosine to form S1P, regulates the balance between sphingolipid metabolites, as it produces the pro-growth, anti-apoptotic S1P and at the same time decreases levels of the pro-apoptotic messengers, ceramide and sphingosine. In normal cells, the activity of sphingosine kinase is low and well controlled. In tumor cell lines and various primary tumors, its expression level is elevated. Sphingosine kinase is also activated by a number of growth and survival factors, including VEGF, PDGF, EGF, FGF, etc. In response to VEGF, high S1P levels promote angiogenesis. Therefore, sphingosine kinase is involved in tumorigenesis, not only because of promotion of cell survival, also because of its effect on neovascularization. Sphingosine kinase may be involved with other pathological states attributed to S1P such as allergic responses, atherosclerosis and other inflammatory related diseases. Two isoforms of sphingosine kinase, SPHK-1 and SPHK-2, are known, as are splice variants such as SPHK-1a and SPHK-1b (see Liu, et al. J. Biol. Chem. 275: 19513-19520 (2000) and Murate, et al. J. Histochem. Cytochem. 49: 845-855 (2001)).

In certain embodiments, the substrate is a spingosine analog. In certain embodiments, the spingosine analogs are selected from: 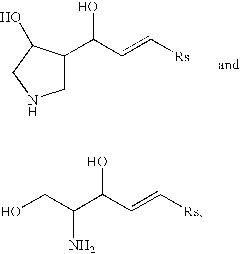
where Rs is alkyl or aryl.

In certain embodiments, Rs is alkyl.

In certain embodiments, the substrate has formula: 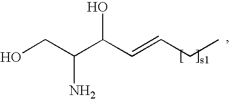
where s1 is 3-20.

In other embodiments, the substrate is sphingosine or D-erythro-sphinganine. In other embodiment, the substrate is a stereoisomers of sphinganine and sphingenine, 1-O-hexadecyl-2-desoxy-2-amino-sn-glycerol, 1-hexadecanol, N-acetyl-D-erythro-sphingenine, 1-amino-2-octadecanol, 2-amino-1-hexadecanol, α-monooleoyl-glycerol, 1-O-octadecyl-rac-glycerol, 1-O-octadecyl-sn-glycerol, and 3-O-octadecyl-sn-glycerol, as described in Gijsbers, S. et al. Biochim. Biophys. Acta 2002, 1580:1-8. Still other substrates are 2-amino-2-[2-(4-octyl-phenyl)-ethyl]-propane-1,3-diol (FTY720) and its analogs such as 2-amino-4-(4-heptyloxy-phenyl)-2-methyl-butan-1-ol (AAL) as described in Kiuchi, et al. J. Med. Chem. 43: 2946-2961 (2000).

In certain embodiments, the substrate is sphingosine.

In certain embodiments, the substrate has formula: 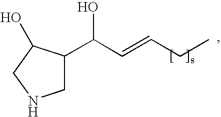
where s is 3-20.

In certain embodiments, the substrate has formula selected from:

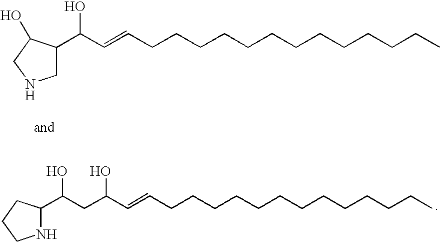

4. Exemplary Conjugates

In certain embodiments, the conjugates provided herein contain a substrate that is a substrate for a peptide kinase and the conjugates have formula:
Sp-L-D

wherein Sp is a natural or non-natural peptide substrate for a protein kinase; L, which may or may not be present, is a non-releasing linker and D is a drug moiety. The drug is non-releasably linked to either the N-terminus or to the carboxy terminus of the peptide. In certain embodiments, the drug is non-releasably linked to the N-terminus of the peptide. In certain embodiments, the drug is non-releasably linked to the carboxy terminus of the peptide.

In certain embodiments, the drug moiety is linked to the carboxy terminus of the peptide substrate for Src. The reactive side chains in the peptide substrate for Src can be free or capped with appropriate capping groups known in the art. The capping groups for the N-terminal amino acids for use herein include, but are not limited to, acetyl, benzoyl, pivaloyl, CBz and BOC. The amino acids containing reactive groups in the side chain, such as Lys and Glu, can be optionally capped with side chain capping groups. Such groups include, acetyl, benzoyl, pivaloyl, CBz, BOC, t-butyl, benzyl and DMAB capping group.

In certain embodiments, the drug moiety is linked to the carboxy terminus of the peptide substrate for Akt. The capping groups for the N-terminal amino acids for use herein include, but are not limited to, acetyl, benzoyl, pivaloyl, CBz and BOC. The amino acids containing reactive groups in the side chain, such as Lys and Glu, can be optionally capped with side chain capping groups. Such groups include, acetyl, benzoyl, pivaloyl, CBz, BOC, t-butyl, benzyl and DMAB capping group.

In certain embodiments, the conjugates contain a drug moiety selected from paclitaxel and vinblastine and a peptide substrate selected from SEQ. ID. Nos. 5, 6, 668, and 1406-1420, linked via a non-releasing linker.

In certain embodiments, the paclitaxel-peptide conjugates contain a non-releasing linker between paclitaxel and the peptide. In certain embodiments, the linker contains an alkylene chain or PEG chain. The linker can be bonded to paclitaxel via a carbamate group at C10 or via an acyl group at C7. In one embodiment, the paclitaxel-peptide conjugates have formula:

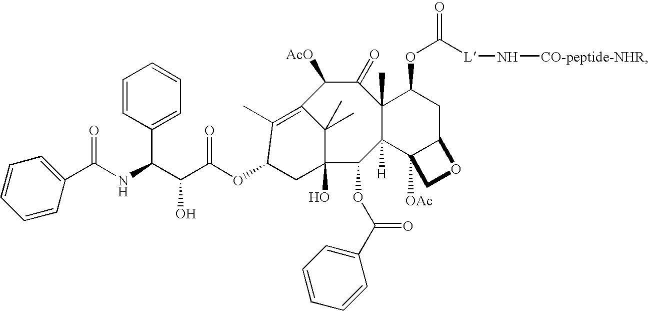

where R is a capping group and where L′ is alkylene or PEG.

In one embodiment, the paclitaxel-peptide conjugates have formula:

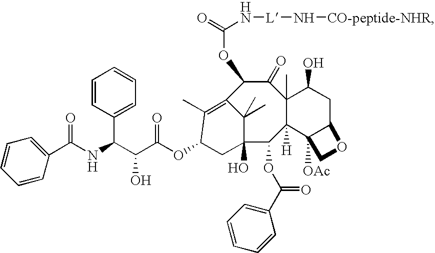

where R is a capping group and where L′ is alkylene or PEG.

In one embodiment, the paclitaxel-peptide conjugates have formula:

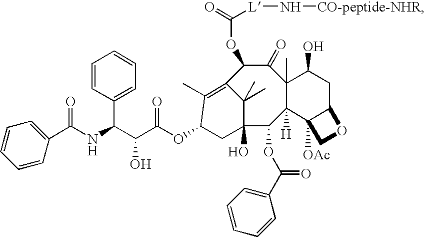

where R is a capping group and where L′ is alkylene or PEG.

In one embodiment, the paclitaxel-peptide conjugates have formula:

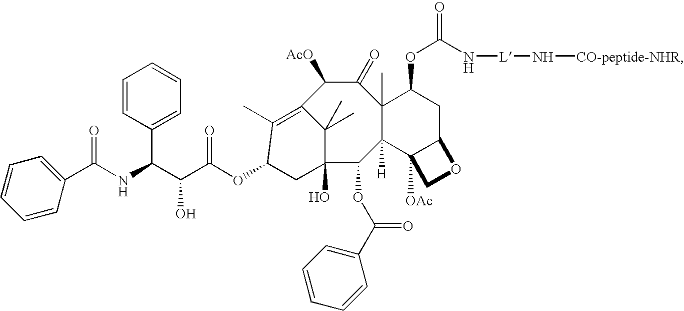

where R is a capping group and where L′ is alkylene or PEG.

In certain embodiments, the conjugates contain a peptide linked to doxorubicin and have formula:

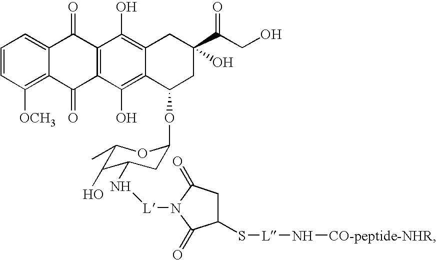

where R is a capping group and where L′ and L″ are each independently alkylene or PEG.

In certain embodiments, the vinblastine-peptide conjuagates provided herein contain an alkylene chain or PEG chain in the linker. The linker can be bonded to vinblastine via an amide group at C3. The peptide substrate in the conjugates is selected from (SEQ ID NOs. 5, 6, 668, and 1406-1420). In one embodiment, the vinblastine-peptide conjugates have formula:

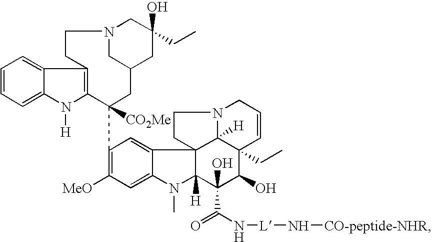

where R is a capping group.

In certain embodiments, the conjugate is selected from

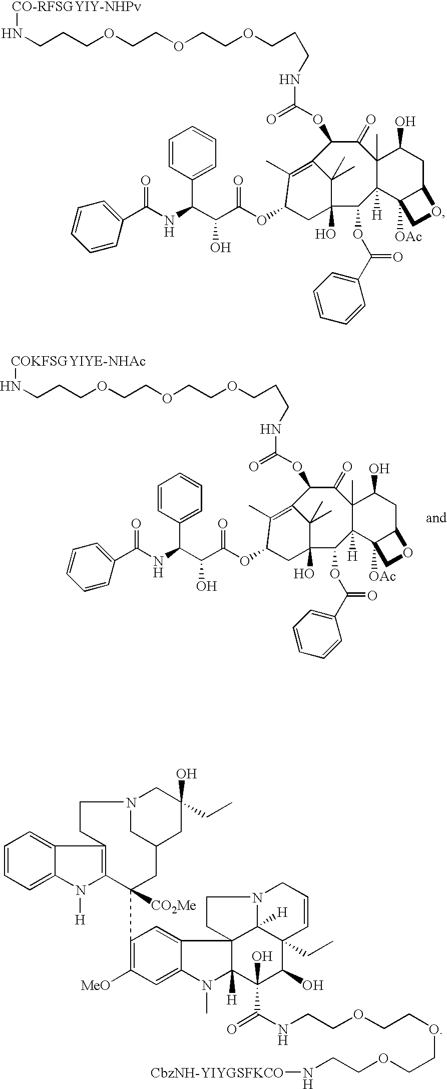

In certain embodiments, the conjugates are vinblastine-sphingosine conjugates. In certain embodiments, the vinblastine-sphingosine conjugates contain a non-releasing linker between vinblastine and sphingosine. In certain embodiments, the linker contains an alkyl chain or PEG chain. In one embodiment, the vinblastine-sphingosine conjugates have formula:

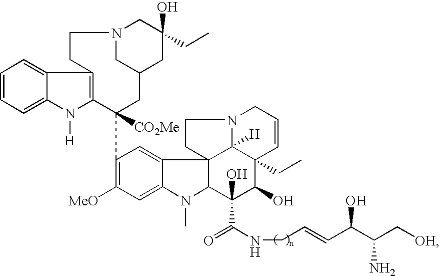

n is 2-10.

In one embodiment, the vinblastine-sphingosine conjugates have formula:

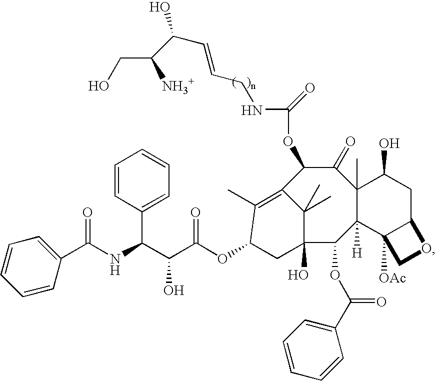

where n is 2-10.

In certain embodiments, the conjugates are anthracycline-sphingosine conjugates. In certain embodiments, the anthracycline-sphingosine conjugates contain a non-releasing linker between anthracycline and sphingosine. In certain embodiments, the linker contains an alkyl chain or PEG chain. In one embodiment, the anthracycline-sphingosine conjugates have formula:

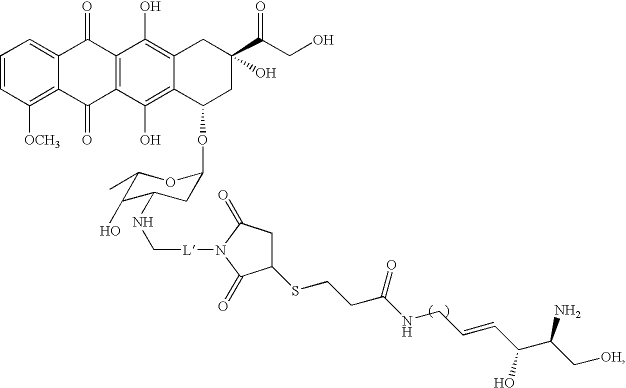

where n is 2-10.

C. Preparation of the Conjugates

The conjugates provided herein can be prepared using any convenient methodology. In one approach, the conjugates are produced using a rational approach. In a rational approach, the conjugates are constructed from their individual components (e.g., drug, linker precursor and substrate). The components can be covalently bonded to one another through functional groups known in the art. Furthermore, the particular portion of the different components modified to provide for covalent linkage will be chosen so as not to substantially adversely interfere with that component's desired binding activity. For example, in a drug moiety, a region that does not affect the target binding activity will be modified, such that a sufficient amount of the desired drug activity is preserved.

The functional groups can be present on the components or introduced onto the components using one or more steps, such as oxidation, reduction, cleavage reactions and the like. Examples of functional groups that can be used in covalently bonding the components to produce the conjugate include but are not limited to hydroxy, sulfhydryl, amino, carbonyl, and the like. Where desirable, certain moieties on the components may be capped using capping groups, as is known in the art, see, e.g., Green & Wuts, Protective Groups in Organic Synthesis (John Wiley & Sons) (1991).

For example, peptides are attached from either their N- or C-terminus directly to a drug or through an intervening linker using a suitable functional group. Scheme 1 illustrates the conjugation of a peptide to a drug where the functional group on the drug for attaching to the peptide is COOH, CHO, halogen, OS(O)2R, NHR, or OH.

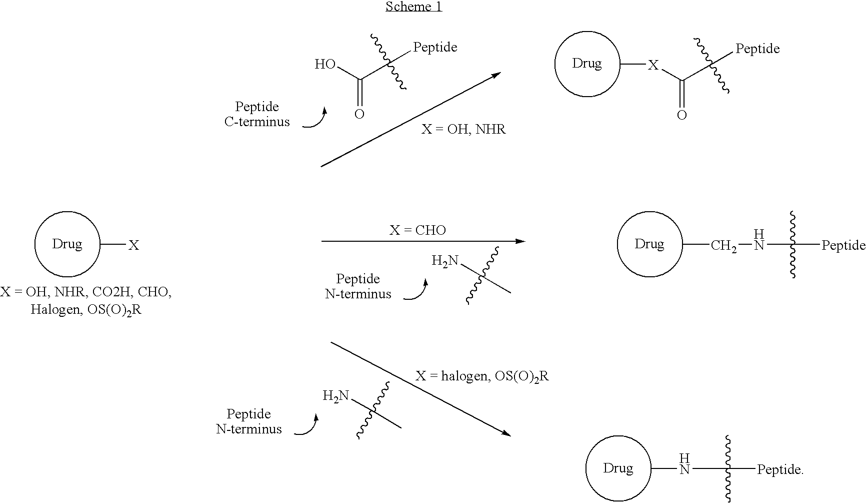

Where COOH is the functional group on the drug, the peptide N-terminus can be attached to the drug using amide bond coupling procedures well known in the art of peptide chemistry. Where CHO is the functional group on the drug, the peptide N-terminus can be attached to the drug by reductive amination using NaBH₄, NaCNBH₄, NaB(OAc)₃H or other suitable reducing groups. Where OH is the functional group on the drug, coupling can be affected by activation of the peptide C-terminus with dicyclohexylcarbodiimide (DCC), or with any other acid activation agent well known in the art for ester bond formation. Where halogen, alkylsulfonyloxy, arylsulfonyloxy, or any other suitable leaving group for nucleophilic displacement is the functional group on the drug is, conjugation may be through nucleophilic displacement by the peptide N-terminus in the presence of Et₃N or any other appropriate acid scavenger.

The same chemical manipulations described above are applicable for attaching a linker precursor to either the C- or N-terminus of the peptide, or for attaching the linker precurser to a functional group on the drug or drug analog. If the drug functionality is OH, then attachment of a linker, either alone or in a Linker-Substrate (L-S) construct, may be made through an ether bond. Drug-Linker (D-L) or Linker-Substrate (L-S) constructs are then chemically combined as illustrated in general by Schemes 2a and 2b.

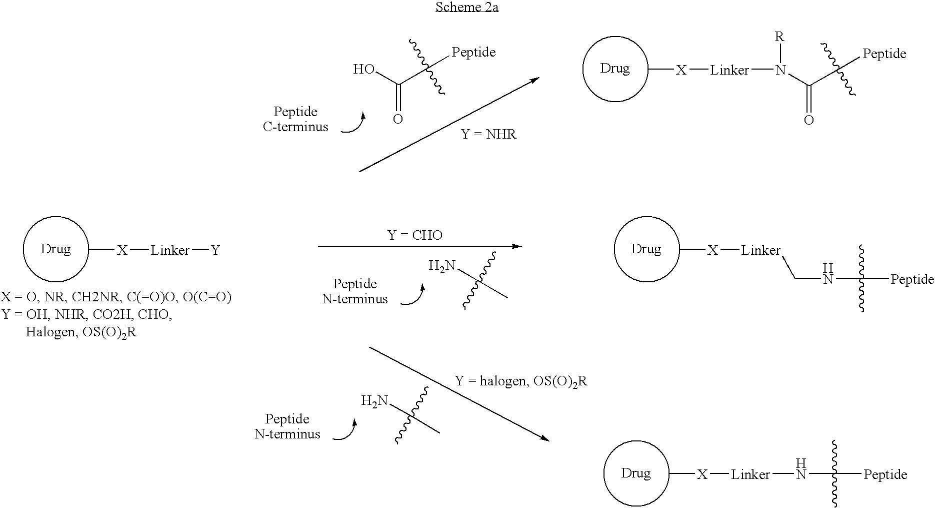

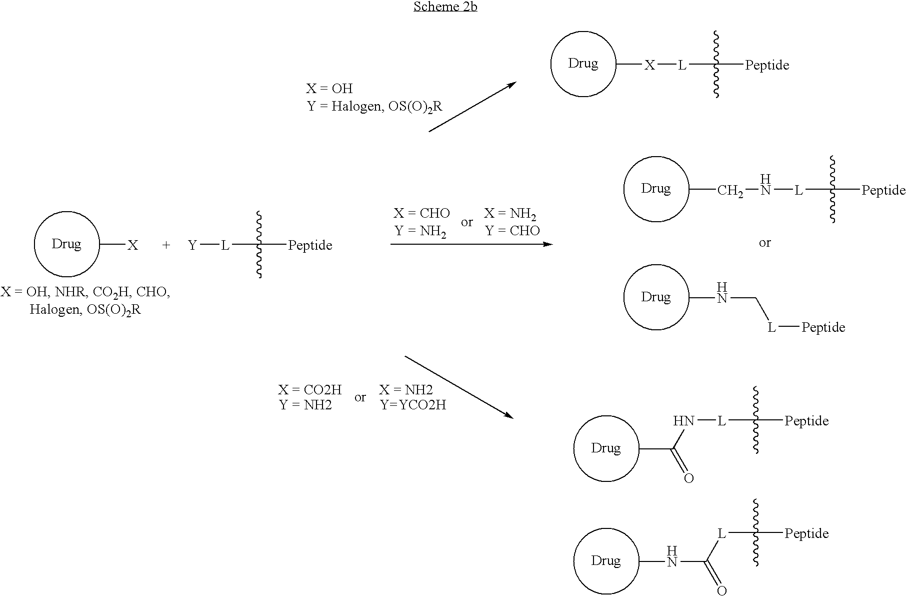

In these schemes, the linker contains a first end and a second end wherein the first end is attached to the drug and the second end is attached to the peptide. The linkers provided herein may contain a subunit which is repeated between 1 and 20 times. Examples of linker units include but are not limited to methylene, ethyleneoxy, a mixture thereof and other applicable suitable linker units.

In another example, the peptide, linker or peptide-linker construct may be attached to the drug through carbamates and ureas as illustrated in Scheme 3.

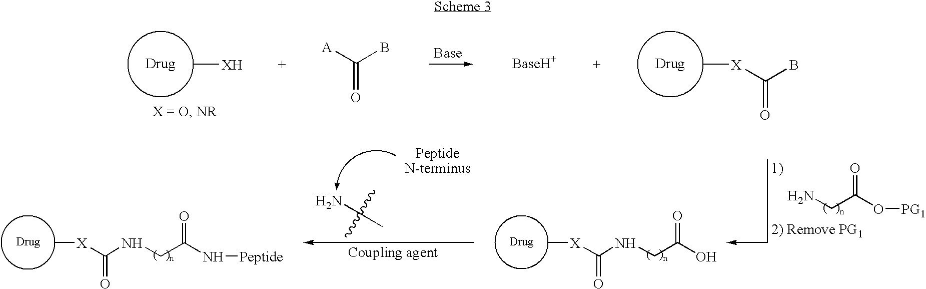

For the carbamate synthesis, the OH or NHR group of the drug or of the linker drug construct may be treated with carbonyl di-imidazole, phosgene or other carbonyl synthon equivalent. The intermediate may then be subsequently treated with an amine either from the free N-terminus of the peptide or the amino group on the linker.

Schemes 4 and 5 illustrate synthetic schemes that can be used for preparing the conjugates provided herein, using paclitaxel as the drug moiety. In Scheme 4a, paclitaxel is protected at the C3′ hydroxyl and condensed with a linker precursor having a carboxylic acid group as a first end and a suitably protected amine as a second end. The repeating unit n, in certain embodiments, is between 1 and 20.

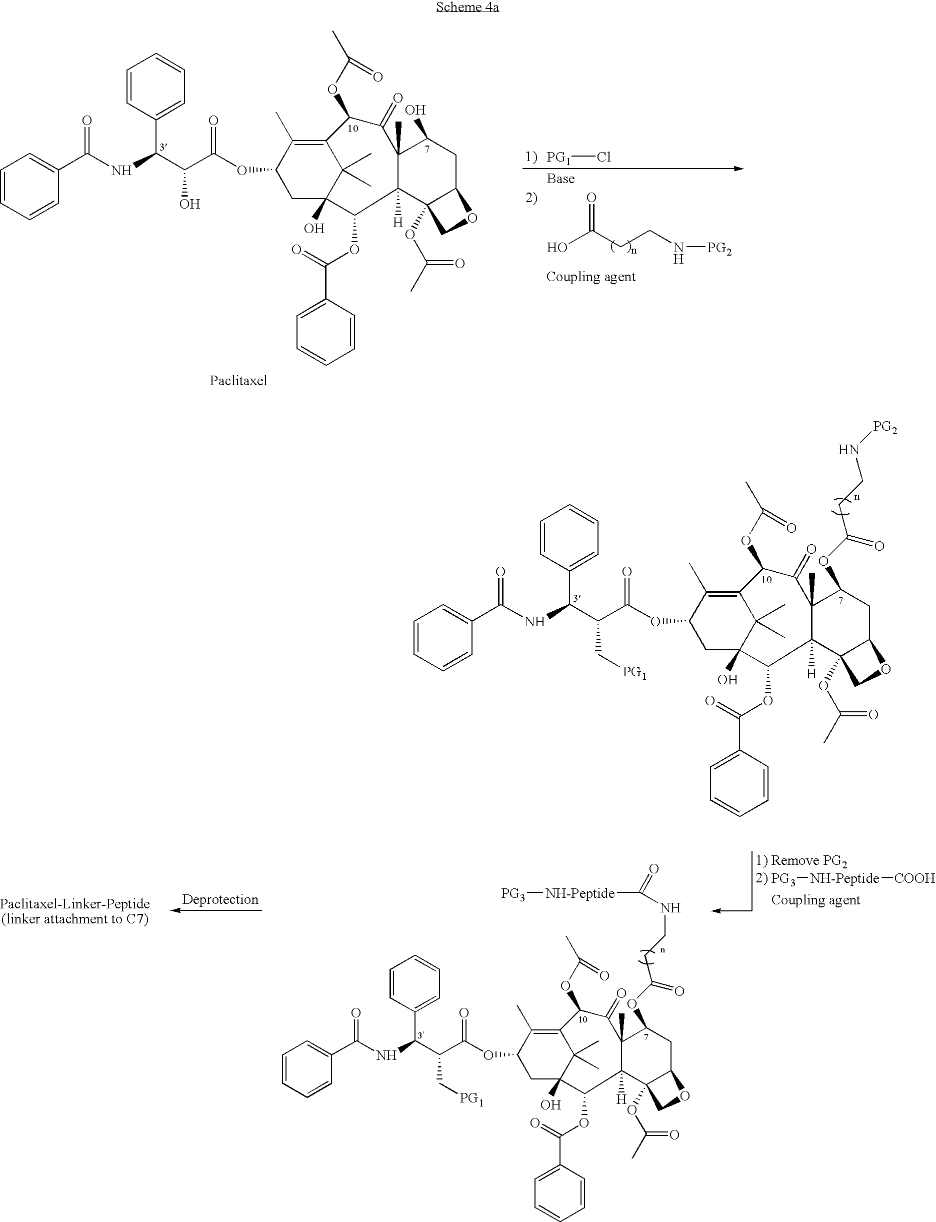

Condensation of the first end to the protected taxane is by DCC or any other appropriate coupling agent used for ester bond formation. Selective removal of the amine protecting group or simultaneous removal of the C3′-OH and amine protecting groups is followed by amide bond formation using standard coupling conditions and an appropriately capped peptide. Deprotection then gives the paclitaxel-linker-conjugate with linker attachment at C7. In Schme 4b, the paclitaxel derivative having a free C10-OH and a protected C7-OH group is condensed with the linker of Scheme 4a to form an ester bond at C10.

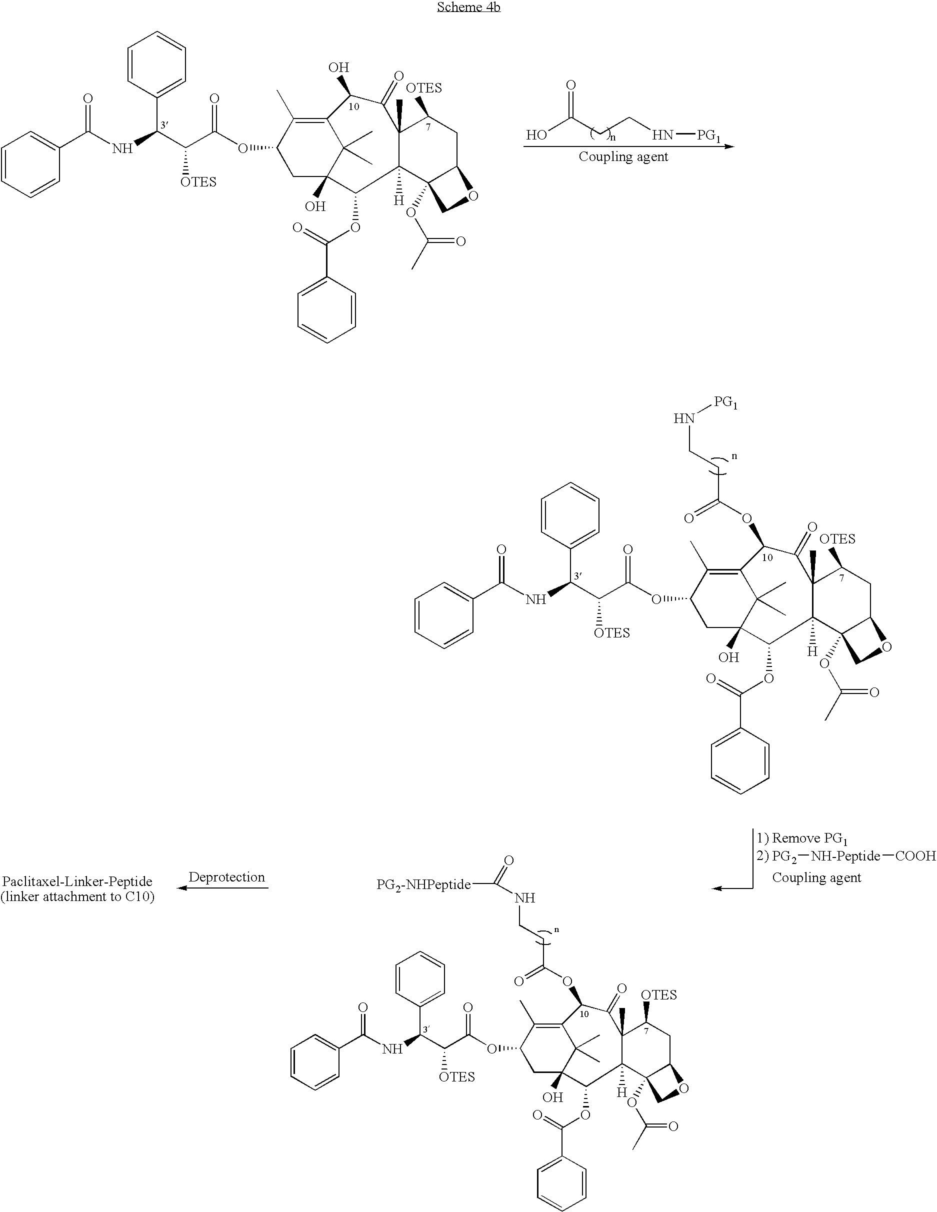

Following the general procedures previously described, the paclitaxel-linker-peptide conjugate with linker attachment at C-10 is obtained.

In Scheme 5a, baccatin III protected at C7 is condensed with an appropriately protected phenylisoleucine to give an intermediate that is deprotected to give the free C3′ amino group.

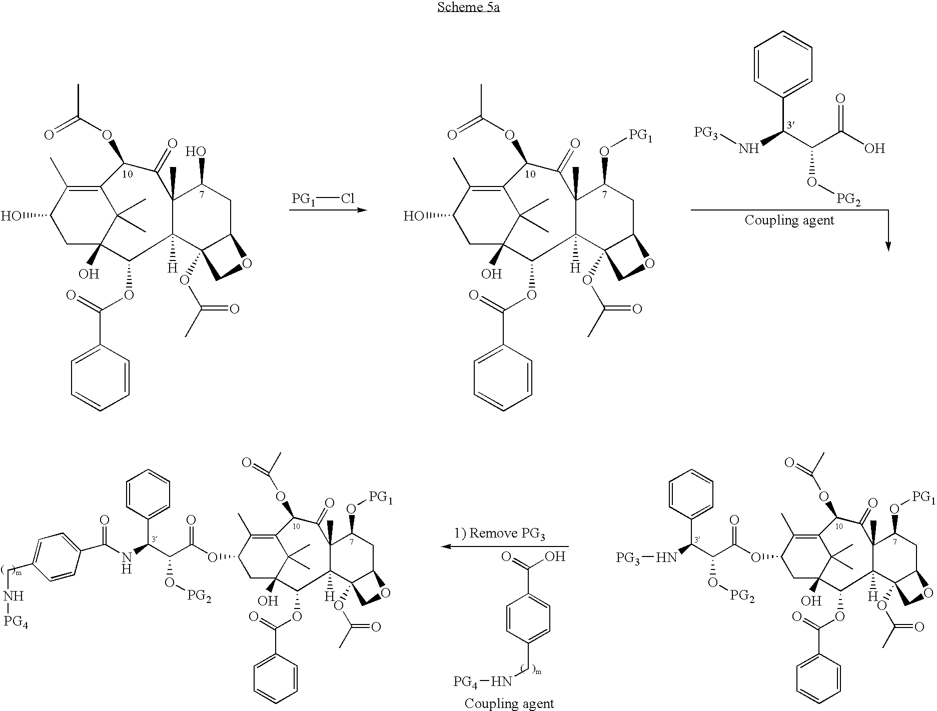

Condensation with a benzoic acid derivative containing a suitably protected amine, wherein m is 0, 1 or 2, provides a paclitaxel derivative with a functional group in the C3′-N benzamido group. Deprotection of the amine followed by peptide coupling and deprotection gives the desired paclitaxel-linker conjugate with attachment at the C3′-N benzamido group (Scheme 5b).

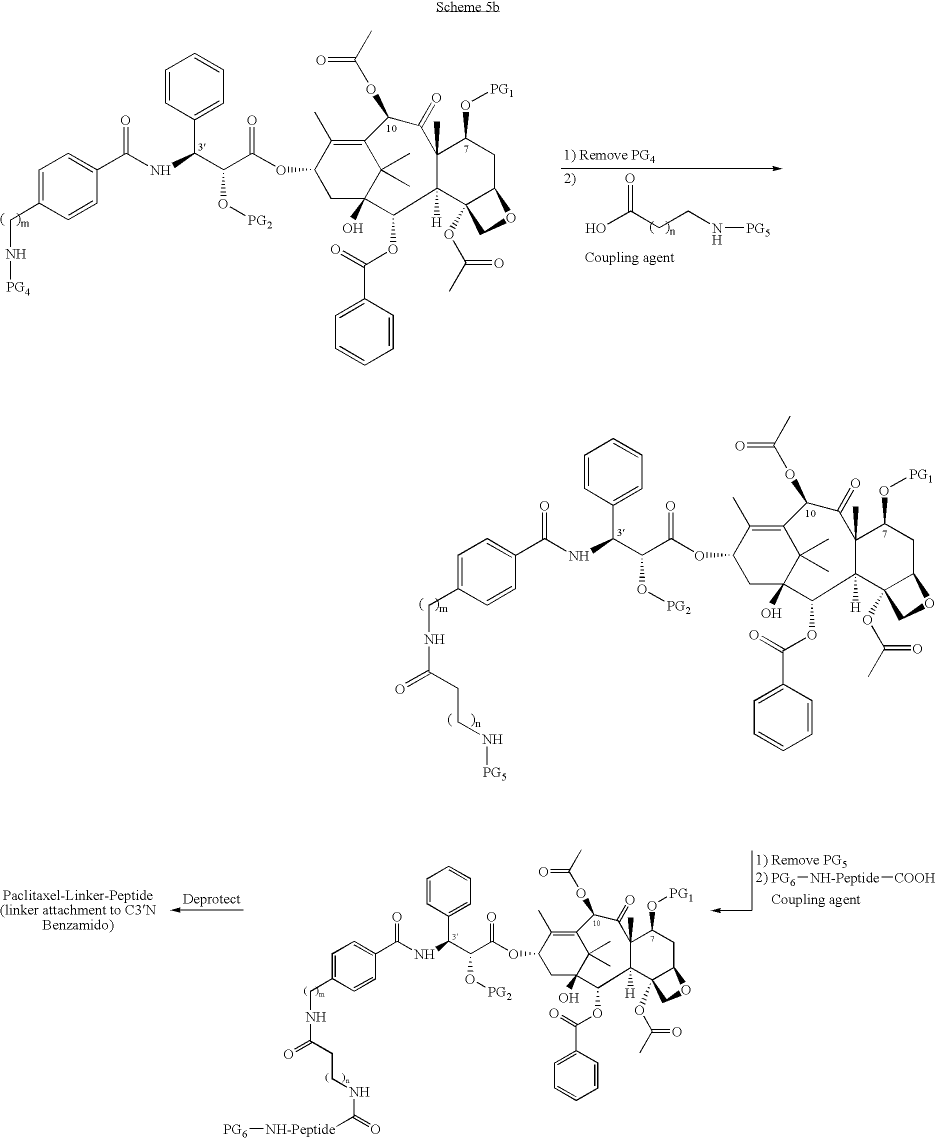

Scheme 6 illustrates a general synthetic scheme for preparing drug-linker-sphingosine conjugates. Sphingosine has been conjugated with fluorescence labels at the end of the linear saturated tridecanyl chain. Pyrene- and NBD-conjugated sphingosine has also been shown to be phosphorylated in vitro with efficiency comparable to the natural substrate. The conjugates appear to be rapidly incorporated and phosphorylated in cultured endothelial cells. NBD-labeled sphingosine conjugate has also been shown to be phosphorylated in vitro and in vivo in cultured CHO cells.

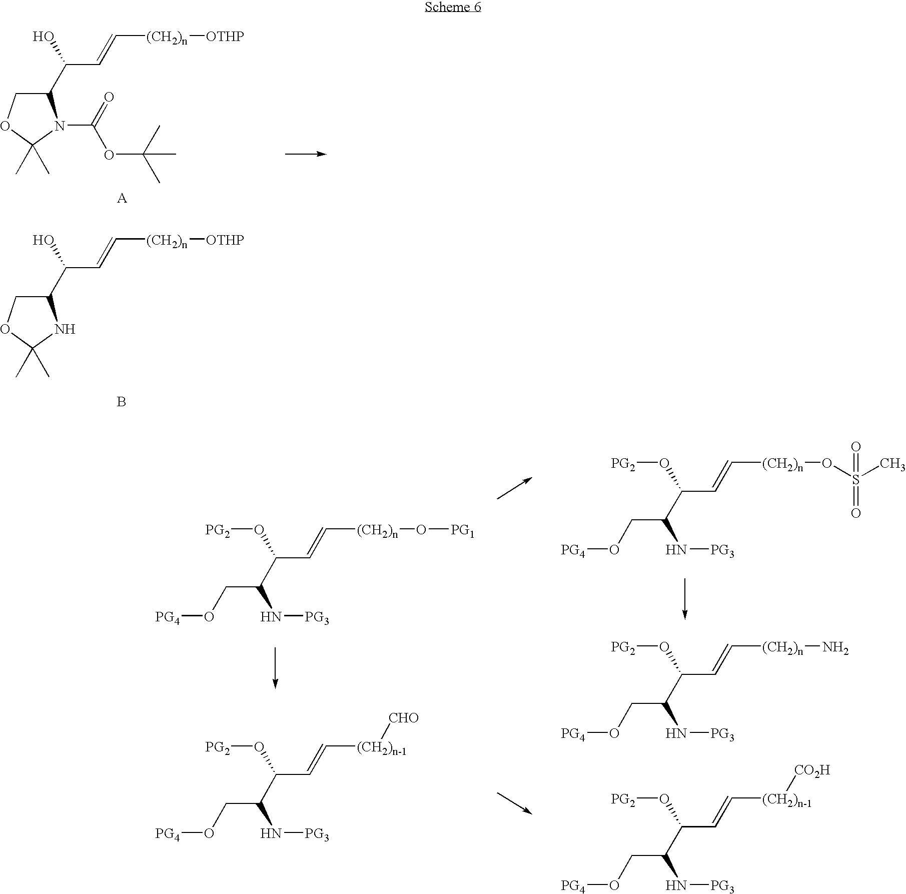

As shown in Scheme 6, sphingosine analogs are prepared with a conserved hydrophilic amino-diol moiety and a 1 to 20 methylene units-long lipid chain with a functional group at the end. The amino-diol moiety may be protected using blocking groups, as is known in the art, see, e.g., Green & Wuts, Protective Groups in Organic Synthesis (John Wiley & Sons, 1991) and they will be removed in the final conjugates. Examples of functional groups at the end of the lipid tail include but are not limited to OH, SH, NH₂, CO₂H, CHO, halo or OS(O)₂R. The drug molecule is prepared with a complementary functional group that will react with the one on sphingosine analog and results in a covalent linkage. A spacer may be inserted between the drug and the functional group so the attached moiety (substrate) is further away from the drug to prevent adverse interference with its desired binding activity. This spacer, in certain embodiments, is 1 to 20 units of methylene or ethyleneoxy and can be attached to the drug through but not limited to ether, amide, carbamate, urea, ester or alkylamine linkage. The routes to sphingosine substrate linker constructs suitable for use in the generalized routes to drug conjugates given in Scheme 1 are exemplified by Scheme 6 starting from known compounds A and B (see Ettmayer, et al. Bioorg. Med. Chem. Let. 14: 1555-1588 (2004) and Hakogi, T., et al. Bioorg. Med. Chem. Let. 13: 661-664 (2003)).

The following reaction schemes further illustrate general methods for the preparation of conjugates provided herein.

### Method for Preparation of Paclitxel C10 carbamates

Existing examples of paclitaxel C-10 carbamates prepared directly from paclitaxel include some simple analogs derived from 10-O-deacetyl-7-O,10-O-bis-[N-(2,2,2-trichloroethyloxy)-aminocarbonyl]-paclitaxel as reported in Bourzat, J. Det al.; EPO Application 524,093 (1993). This synthetic methodology, however, is not versatile since selective reaction of the amine input at C-10 is possible only in dichloromethane. A more general approach for the synthesis of C-10 carbamates starts from 10-deacetyl-baccatin-III. However, subsequent steps to install the phenylisoserine side chain are problematic for amine inputs containing additional functional groups that require protection. Due to the chemical sensitivity of the taxane core, the protecting group strategy required for such amine inputs would be complex. Disclosed in the instant application is a method which permits the use of amine inputs containing additional functionality in free form. The disclosed method allows for the syntheses of C10 carbamates directly from paclitaxel that otherwise would be inaccessible or difficult to prepare.

A procedure for preparation of Paclitaxel C10 carbamates as provided herein is illustrated in Schemes 7 and 8. Accordingly, compound 5a can be converted in nearly quantitative yield into its C10 carbonylimidazole 6a by reaction with carbonyl-diimidazole (CDI) in dichloromethane at room temperature. Compound 6a can be reacted with amines in suitable solvents to yield the corresponding carbamate 8a, which can be deprotected to give 9a. Typically, for primary and secondary amines, the reaction can be carried out in non-polar solvents, such as dichloromethane or in protic solvents such as IPA or t-BuOH at elevated temperatures. 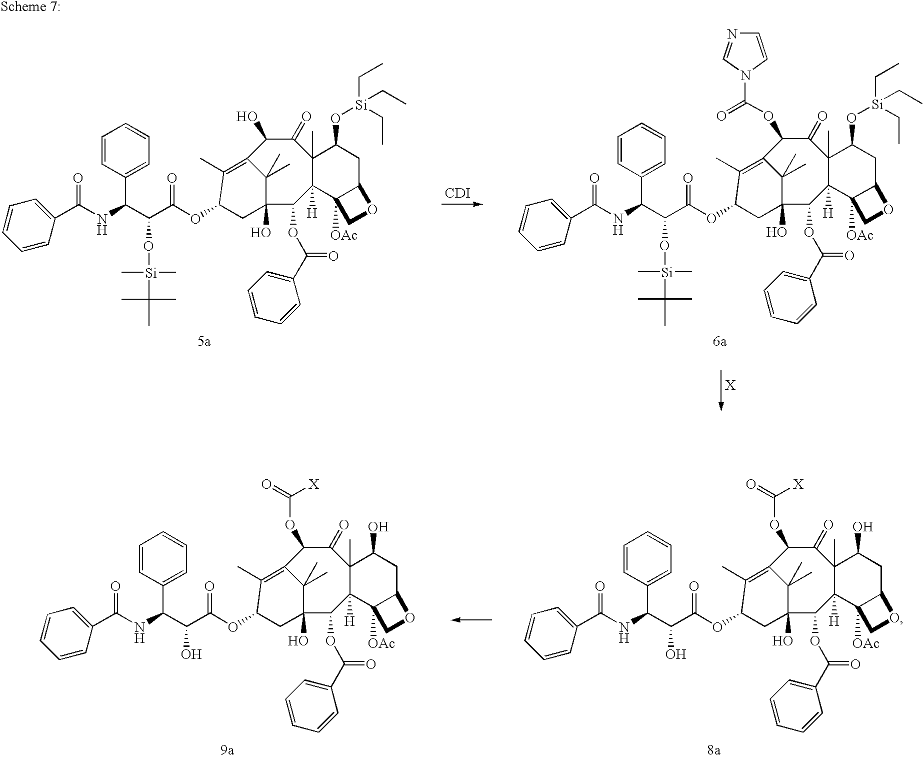
where X is an amine.

In certain embodiments, the C10-carbonylimidazole 6a can be activated with an alkylating agent such as an alkyl halide, alkyl sulfonate or di-alkyl-sulfate to give a N¹-alkyl-N³-acyl imidazolium species represented by 7a of Scheme 8. In certain embodiments the alkylating agent is selected from dimetylsulfate and methyl iodide. The imidazolium species can then be reacted with various amines either in free or salt forms in protic solvents or aprotic solvents such as DMF, DMSO or dioxane. For amine salts condensation with 7a is conducted in the presence of a hindered base such as DIEA. In certain embodiments, less reactive amines, such as arylamines or heteroarylamines may be condensed with 7a to obtain paclitaxel C10 carbamates with N-aryl or N-heteroaryl linker attachment.

Various nucleophiles can be used in the reactions provided herein to prepare C10 paclitaxel carbamates. Certain exemplary nucleophiles include, but are not limited to, primary and secondary amines, amine containing acids, such as α-amino acids, amino-sugars, such as glucosamine, arylamines, heteroarylamines, and α,α-disubstituted alcohols.

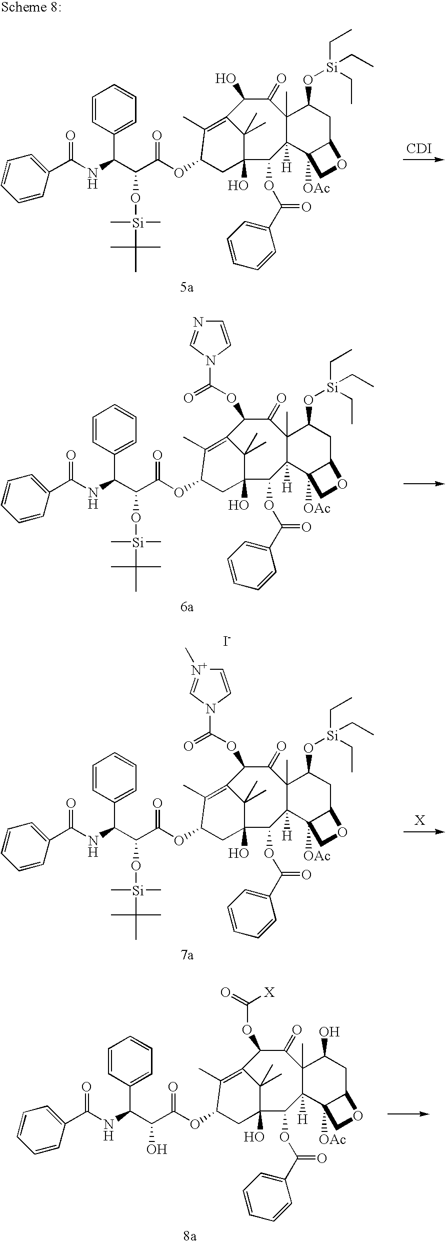

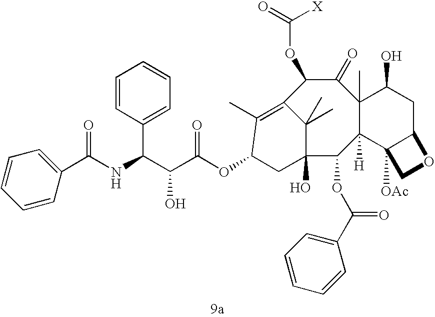

The following reaction schemes illustrate general methods for the preparation of conjugates provided herein

An exemplary preparation of paclitaxel-linker-peptide conjugate with C10 as point of attachment is described herein.

The following description and reaction schemes provide general methods for preparation of conjugates provided herein.

### a. Preparation of a Paclitaxel-Linker-Peptide Conjugate (4)

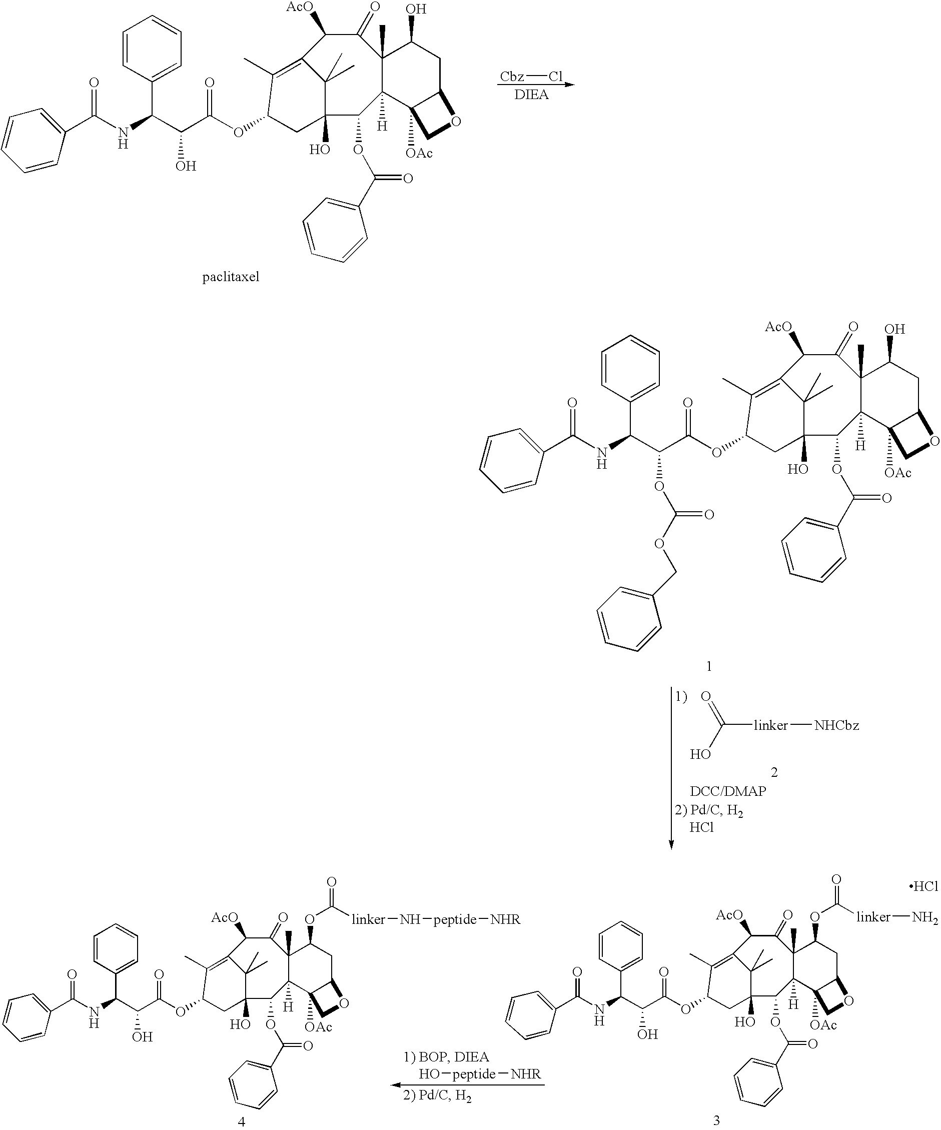

### Preparation of 2′-benzyloxycarbonyl-paclitaxel (1)

Benzyl chloroformate is added to a solution of paclitaxel in DCM followed by DIEA. After stirring for 16 h the reaction mixture is concentrated and the resulting residue was purified by silica gel chromatography eluting with 1:1 hexanes:ethyl acetate to give the title compound.

### Reaction of 2′-benzyloxycarbonyl-paclitaxel with N-protected amine containing acids

To a Cbz protected amine containing acid of general formula 2 (160 mol %) and 2′-benzyloxycarbonyl-paclitaxel (1, 100 mol %) in DCM at 0° C. is slowly added a DCM solution of DCC (200 mol %) and a catalytic amount of DMAP. The reaction mixture is stirred for 16 h and allowed to reach room temperature. The reaction mixture is then filtered and the volatiles removed under reduced pressure. The residue so obtained is purified by silica gel chromatography eluting with a hexanes-ethyl acetate mixture to give a Cbz-protected paclitaxel-linker-amine intermediate. Removal of the Cbz group is conducted in a 7:3 mixture of THF:water using a catalytic amount of 10 wt % palladium on carbon and HCl (100 mol %, introduced as a 1 M aqueous solution), with shaking for 1.5 hours under 60 psi H₂. Filtration over Celite, concentration under reduced pressure and lyophilization provides a paclitaxel-linker-amine intermediate of general structure 3.

### Preparation of paclitaxel-linker-peptide conjugates with acyl linker attachment to C7 of paclitaxel

To a paclitaxel-linker-amine intermediate (3, 100 mol %) and a suitably protected peptide (100 mol %) in DMSO are added BOP (100 mol %) and DEA (200 mol %). The reaction mixture is stirred for 16 h and directly injected onto a preparative RP-HPLC C-18 column for purification (Method A). Fractions containing the appropriate mass, as determined by analytical HPLC-MS (Method B), are pooled and CH₃CN is removed under reduced pressure or N₂ stream. The remaining aqueous mixture is then lyophilized to yield a paclitaxel-linker-peptide conjugate of general structure 4 in 10-20% yields. Protecting group(s) on the peptide are removed to provide additional paclitaxel-linker-peptide conjugates using catalytic hydrogenation conditions typically employing 10 wt % palladium on carbon in CH₃OH under an atmosphere of hydrogen.

### b. Preparation of a Paclitaxel-Linker-Peptide Conjugate of Formula 9

In certain embodiments, the Paclitaxel-Linker-Peptide Conjugates containing a linker conjugated to paclitaxel via a carbamate functionality at C10 can be prepared by the procedure illustrated in Scheme 7.

Preparation of paclitaxel-2′-(tert-butyldimethylsilyl)-7-(triethylsilyl)-10-(deacetyl-carbonylimidazole) (6)

To 10-deacetyl-2′-(tert-butyldimethylsilyl)-7-(triethylsilyl)-paclitaxel prepared according to the procedure in Datta, A.; Hepperle, M. I. G., J. Org. Chem. (1995) 60:761, in anhydrous DCM is added CDI (400 mol %). The reaction mixture is allowed to stir for 16 hours at room temperature under nitrogen atmosphere then extracted with water (5 mL). The organic layer is dried over sodium sulfate, filtered and concentrated to give the title compound 6 which is subsequently used without purification.

### Reaction of paclitaxel-2′-(tert-butyldimethylsilyl)-7-(triethylsilyl)-10-(deacetyl-carbonylimidazole) with mono-protected diamines

To paclitaxel-2′-(tert-butyldimethylsilyl)-7-(triethylsilyl)-10-(deacetylcarbonyl-imidazole) (6, 100 mol %) dissolved in anhydrous isopropyl alcohol is added a mono-Cbz protected diamine (300 mol %) of formula 7. The reaction mixture is stirred under reflux for 16 hours. The volatiles are then removed in vacuo and the resulting residue is re-dissolved in DCM. The organic solution is then extracted with water and dried over sodium sulfate. After filtration and evaporation of the volatiles the residue is desilylated following the procedure in Ojima, I. et al., J. Med. Chem. (1997) 40:267. The residue so obtained is dissolved in a 7:3 mixture of THF:water, whereupon 10 wt % palladium on carbon and HCl (100 mol %, introduced as a 1 M aqueous solution), is added. The resulting mixture is shaken for 3 hours under 60 psi of H₂. The reaction mixture is filtered through Celite and concentrated under reduced pressure and lyophilized. The residue so obtained is purified by preparative RP-HPLC (Method A). Fractions containing the appropriate mass, as determined by analytical HPLC-MS (Method B) are pooled and CH₃CN removed under reduced pressure. The remaining aqueous mixture is then lyophilized obtaining a desired paclitaxel-10-deacetyl, 10-carbamoyl-linker-amino intermediate of general structure 8.

### Preparation of paclitaxel-linker-peptide conjugates with carbamate linker attachment at paclitaxel C10

To a paclitaxel-10-deacetyl,10-carbamoyl-linker-amine (8, 100 mol %) dissolved in DMSO is added a suitably protected peptide (100 mol %) followed by BOP (100 mol %) and DIEA (200 mol %). The reaction mixture is stirred for 16 h then directly injected onto a preparative RP-HPLC C-18 column for purification (Method A). Fractions containing the appropriate mass, as determined by analytical HPLC-MS (Method B) are pooled and CH₃CN is removed under reduced pressure. The remaining aqueous mixture is then lyophilized to give a paclitaxel-linker-peptide conjugate of general formula 9. Protecting group(s) on the peptide are removed to provide additional paclitaxel-linker-peptide conjugates using catalytic hydrogenation conditions typically employing palladium on carbon in CH₃OH under an atmosphere of hydrogen.

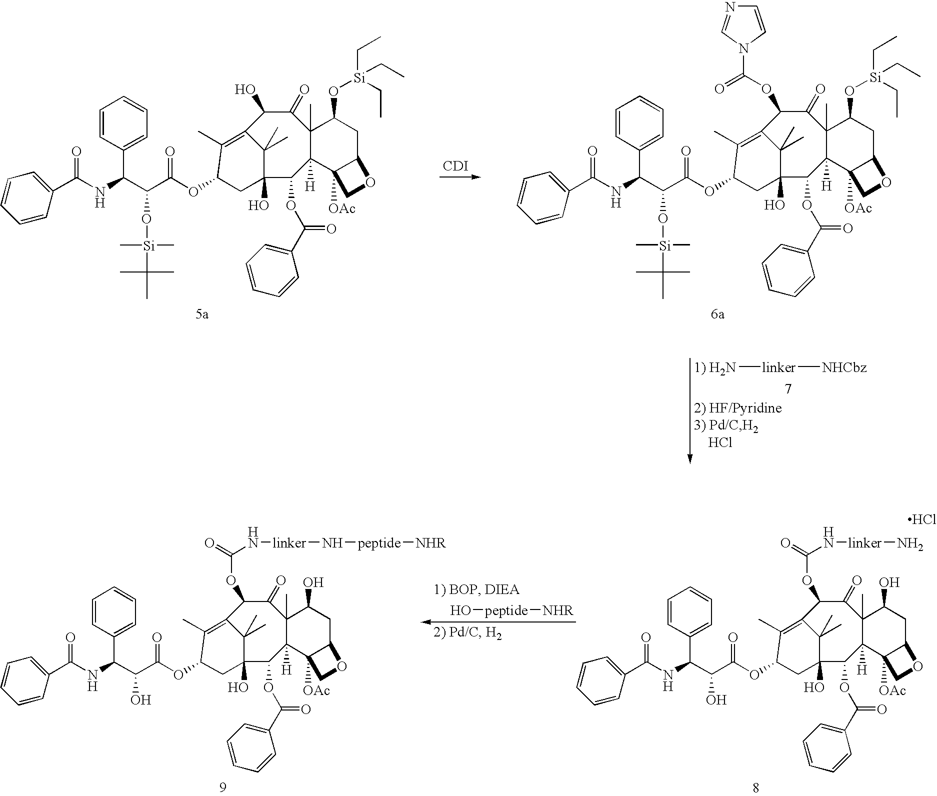

### c. Preparation of a Paclitaxel-Linker-Peptide Conjugate of Formula 12

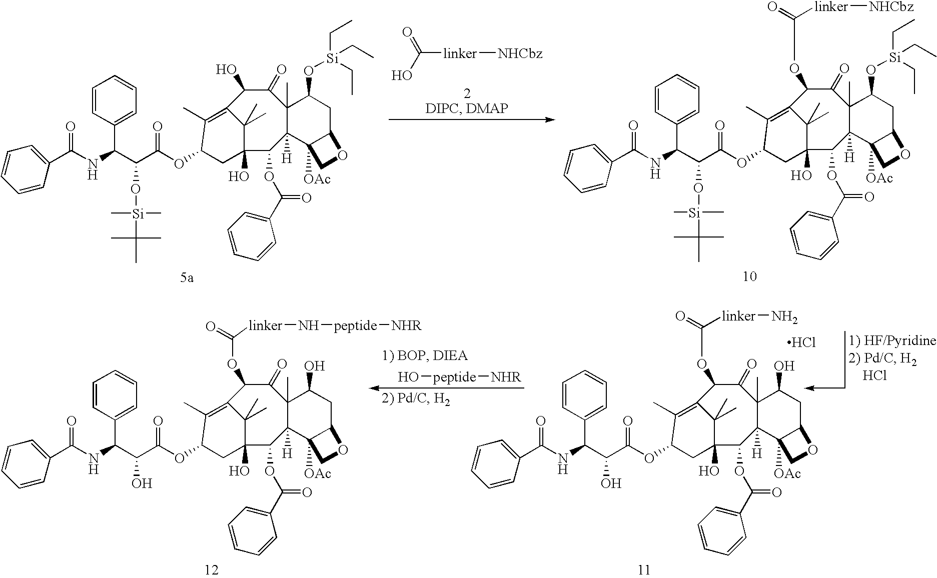

To 10-deacetyl-2′-(tert-butyldimethylsilyl)-7-(triethylsilyl)paclitaxel (5a), 100 mol %) prepared according to the procedure in Datta, A.; Hepperle, M. I. G., J. Org Chem. (1995) 60:761, and DMAP (200 mol %) dissolved in anhydrous toluene is added to a previously prepared solution of a N-Cbz protected amine containing acid (2, 600 mol %), DIPC (600 mol %) in anhydrous toluene. The reaction mixture is then stirred at 70° C. for 100 hours under nitrogen atmosphere. The reaction mixture is then diluted with ethyl acetate, extracted with sodium bicarbonate (5% aqueous solution) and brine. The organic layer is then dried over sodium sulfate. After filtration and evaporation of the volatiles, the residue is purified by silica gel chromatography eluting with 7:3 hexanes:ethyl acetate to give the paclitaxel-2′-(tert-butyldimethylsilyl)-7-(triethylsilyl)-10-deacetyl, 10-acyl-linker of general structure 10 in 49% yield. Desylilation of 10 according to the procedure described in Ojima, I., et al., J. Med. Chem. (1997) 40:267 is followed by catalytic hydrogenation using a 7:3 mixture of tetrahydrofuran:water, with 10 wt % palladium on carbon and HCl (100 mol %, added as a 1 M aqueous solution) with shaking for 3 hours under 60 psi of H₂. The resulting reaction mixture is filtered through Celite and the volatiles were removed in vacuo. The residue is purified by silica gel chromatography eluting with 1:2 hexanes:ethyl acetate to give a 10-deacetyl-paclitaxel-linker-amine intermediate of general structure 11.

### Preparation of 10-deacetyl-paclitaxel-linker-peptide conjugates with acyl linker attachment at paclitaxel C10

To a 10-deacetyl-paclitaxel-linker-amine (11, 100 mol %), in DMSO is added a suitably protected peptide (100 mol %) followed by BOP (100 mol %) and DIEA (200 mol %). The reaction mixture is stirred for 16 h then directly injected onto a preparative RP-HPLC C-18 reversed phase column for purification (Method A). Fractions containing the appropriate mass, as determined by analytical HPLC-MS (Method B), are pooled and CH₃CN was removed under reduced pressure or N₂ stream. The remaining aqueous mixture is then lyophilized to yield a paclitaxel-linker-peptide conjugate of general structure 12 in 30-40% yields. Protecting group(s) on the peptide are removed to provide additional paclitaxel-linker-peptide conjugates using catalytic hydrogenation conditions typically employing Palladium on carbon in CH₃OH under an atmosphere of hydrogen.

### d. Preparation of Paclitaxel-Linker-Peptide Conjugates with Carbamate Linker Attachment at Paclitaxel C7

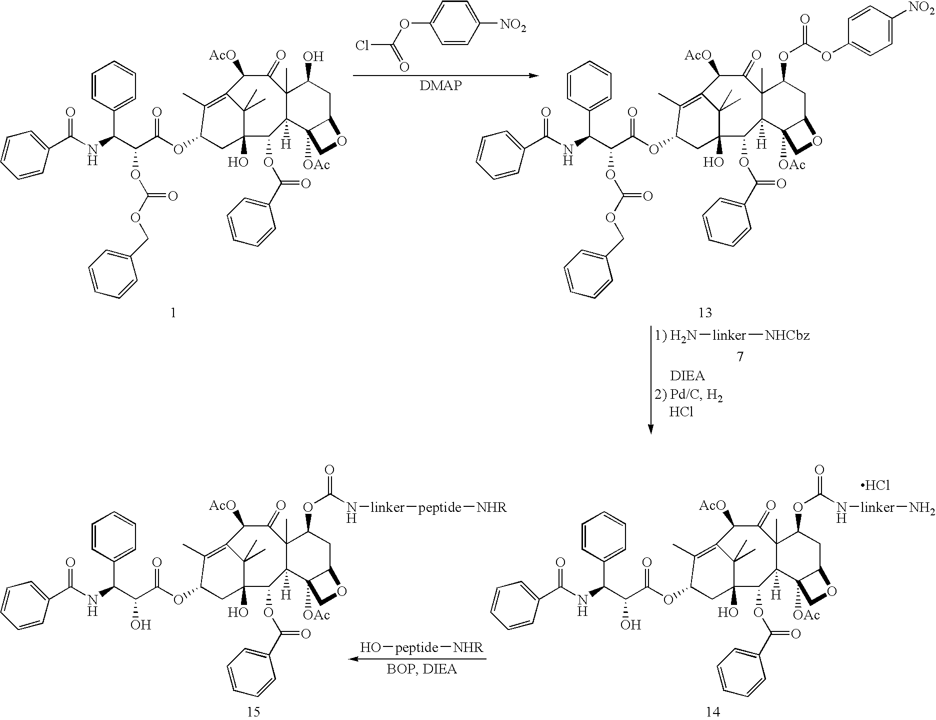

To 2′-(benzyloxycarbonyl)-paclitaxel (1), prepared as described elsewhere herein, dissolved in methylene chloride are added p-nitrophenylchloroformate and DMAP. The reaction mixture is stirred for 1 h and concentrated to dryness. The resulting residue is purified by silica gel chromatography column eluting with 1:1 hexanes:ethyl acetate to give (13).

### Reaction of 7-(p-nitrophenylcarbonyl)paclitaxel with 10 mono-protected diamines

To 2′-(benzyloxycarbonyl)-paclitaxel, 7-(p-nitrophenylcarbonyl)paclitaxel (13, 100 mol %) and a mono Cbz-protected diamine (7, 100 mol %) dissolved in DCM is added neat, or as a DMF, or DCM solution followed by DIEA (1000 mol %). The reaction mixture is stirred for 90 min then partitioned between ethyl acetate and water. The aqueous layer is extracted with ethyl acetate and the organic layer is dried over Na₂SO₄ and concentrated to dryness to give a residue which is purified by silica gel chromatography. The 2′-benzyloxypaclitaxel(C7-carbamoyl)-linker intermediate so obtained is subjected to catalytic hydrogenation using CH₃OH and HCl (200 mol %, introduced as a 1 M aqueous solution) with 10 wt % palladium on carbon and stirring under 60 psi atmosphere of H₂ for 5 h. Filtration of the reaction mixture on Celite, removal of volatiles in vacuo and lyophilization provided the paclitaxel(C7-carbamoyl)-linker-amine intermediate of general structure 14.

### Preparation of paclitaxel-linker-peptide conjugates with carbamate attachment at paclitaxel C7

To paclitaxel-(C7-carbamoyl)-linker-amine (14, 100 mol %) and a suitably protected peptide (100 mol %) in DMSO are added BOP (100 mol %) and DEEA (200 mol %). The reaction mixture is stirred for 16 h whereupon the reaction mixture is directly injected onto a preparative RP-HPLC C-18 reversed phase column for purification (Method A). Fractions containing the appropriate mass, as determined by analytical HPLC-MS (Method B), are pooled and CH₃CN is removed under reduced pressure or N₂ stream and the aqueous mixture is lyophilized to give paclitaxel-linker-peptide conjugate of general structure 15. Protecting group(s) on the peptide are removed to provide additional paclitaxel-linker peptide conjugates using catalytic hydrogenation conditions typically employing 10 wt % palladium on carbon in CH₃OH under an atmosphere of hydrogen.

### e. Preparation of Deacetyl-Vinblastine-Linker-Peptide Conjugates with Amide Linker Attachment at C3 of Vinblastine

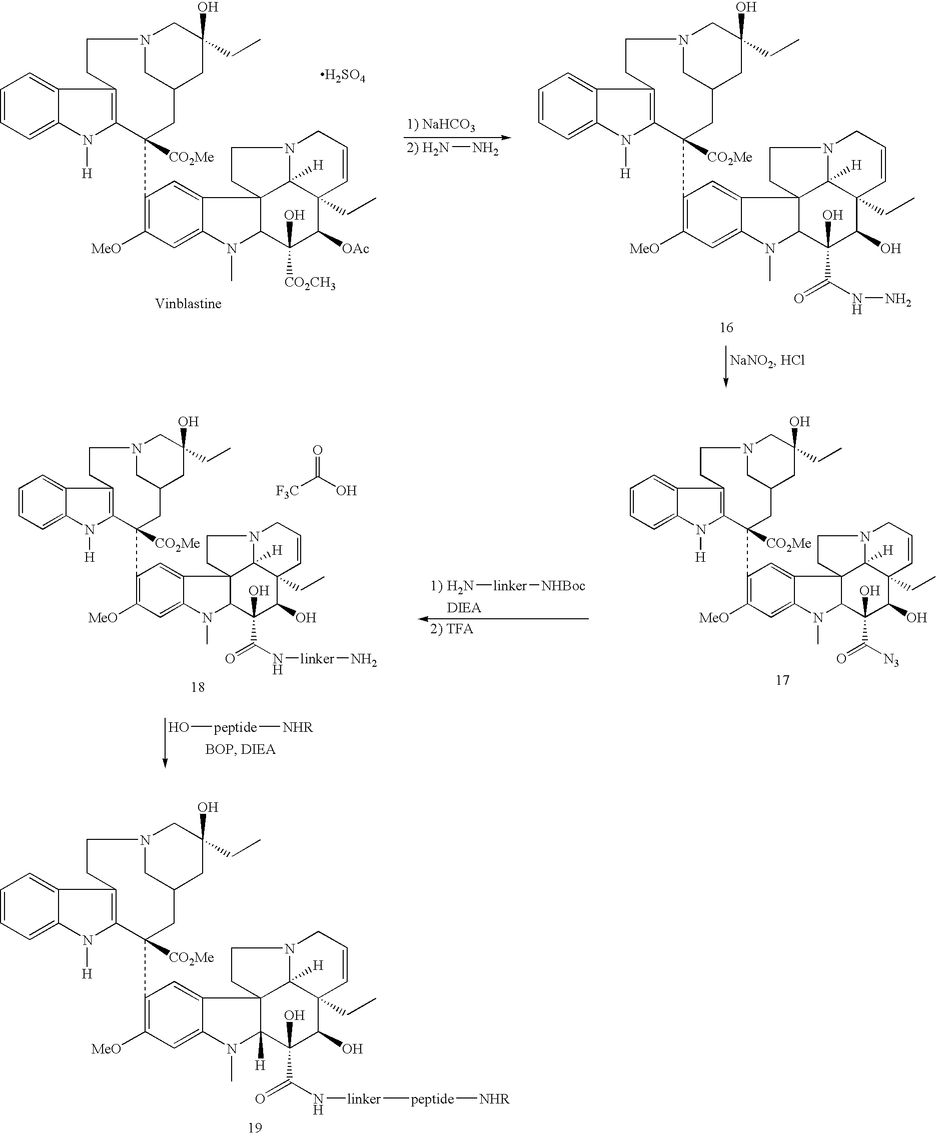

### Synthesis of deacetylvinblastine acid azide (17)

Deacetylvinblastine monohydrazine (16) prepared according to the procedure described in. Bhushana, K. S. P Rao, et al., J. Med. Chem. (1985) 28:1079 is dissolved in a mixture of CH₃OH (20 mL) and an aqueous 1 M HCl solution (50 mL). The solution is cooled to −10° C. and then NaNO₂ is added at once with stirring. After 10 min the pH of the brownish-red solution is adjusted to 8.8 with a saturated aqueous sodium bicarbonate solution and is extracted rapidly with DCM and washed with a saturated aqueous NaCl solution. The extracts are dried over Na₂SO₄ and concentrated to a volume of 50 mL. The solution of deacetylvinblastine acid azide (17) is used directly in the next step.

### Reaction of deacetylvinblastine acid azide with mono-protected diamines

To a solution of deacetylvinblastine acid azide (17) is added neat, or in a solution of DCM or DMF a mono Boc-protected diamine (150 mol %) followed by DIEA. The reaction mixture is stirred for 3 h then concentrated in vacuo to give a residue that is purified by silica gel chromatography to give a Boc-protected deacetylvinblastinyl-linker-amine. Removal of the Boc group is effected with a 1:1 mixture of DCM:TFA with stirring for 10 min. Concentration to dryness with a stream of N₂ and lyophilization gave a deacetylvinblastine-linker-amine of general structure 18.

### Preparation of a vinblastine-linker-peptide conjugate with amide linker attachment at vinblastine C-3

To a deacetylvinblastinyl-linker-amine-TFA (18, 100 mol %) and a suitably protected peptide (100 mol %) in DMSO are added BOP (150 mol %) and DIEA (400 mol %). The reaction mixture is stirred for 4 h and then directly injected onto a preparative RP-HPLC C-18 reversed phase column for purification (Method A). Fractions containing the appropriate mass, as determined by analytical HPLC-MS (Method B), are pooled and CH₃CN is removed under reduced pressure or N₂ stream and the remaining aqueous mixture is lyophilized to give vinblastine-linker-peptide conjugate of general structure 19. Acid sensitive protecting group(s) on the peptide are removed to provide additional vinblastine-linker-peptide conjugates by treatment with 1:1 DCM:TFA, for 10 min, followed by concentration and lyophilization. Base sensitive protecting groups are removed using piperidine or a 2% hydrazine solution in DMF.

### f. Preparation of a Doxorubicin-Linker-Peptide with Alkyl Linker Attachment at C3′-N

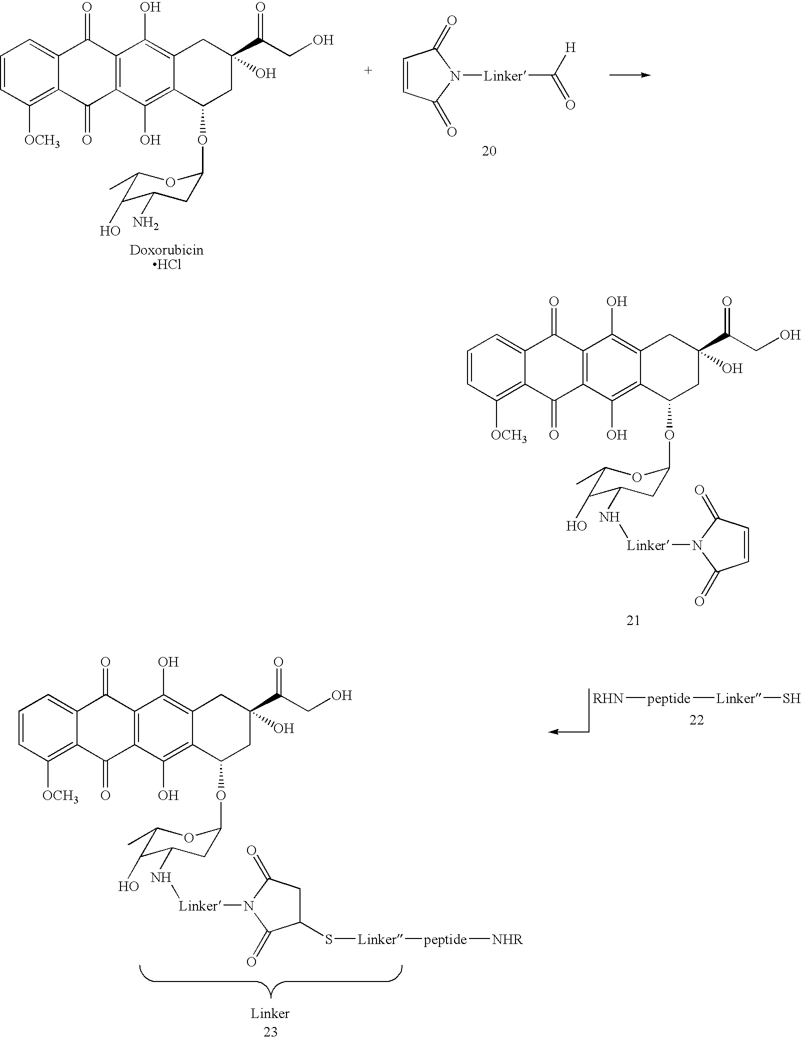

### Preparation of 3-(2,5-dioxo-2,5-dihydropyrrol-1-yl)propionaldehyde (20)

To 1-(3-hydroxypropyl)-1H-pyrrole-2,5-dione dissolved in DCM, DMP is added in one portion. After stirring the mixture for 2 h, 2-propanol is added followed by stirring for an additional 30 min. The resulting solution is filtered through a silica gel pad eluted with EtOAc, and the filtrate is concentrated. The crude product is purified by silica gel chromatography eluting with EtOAc/Hexane (2/1) to provide 3-(2,5-Dioxo-2,5-dihydro-pyrrol-1-yl)-propionaldehyde.

### Preparation of an anthracycline-maleimide intermediate with N-alkyl attached to 3′-N of the anthracycline

To a stirred solution of doxorubicin hydrochloride, an aldehyde-maleimide intermediate (20, 200-300 mol %) and glacial AcOH (20 μL, 195 mol %) in CH₃CN/H₂O (2:1) is added a 1 M solution of NaCNBH₃ in THF (0.33 mol %). The mixture is stirred under nitrogen atmosphere in the dark at RT for 1 h. The solution is then concentrated under vacuum to give a residue which is diluted with an aqueous 5% NaHCO₃ solution and extracted with DCM. Concentration of the organic solution and purification of the resulting residue by silica gel chromatography eluting with DCM/CH₃OH (20:1) provided the anthracycline-maleimide intermediate of general structure 21.

### Preparation of a peptide of Formula 22 suitable for reaction with the alkyl anthracycline-maleimide intermediate

To a suitably protected peptide with a free C-terminal (100 mol %) in DMF is added BOP (100 mol %), DIEA (400 mol %) and H₂NCH₂CH₂SH hydrochloride salt (100 mol %). The reaction mixture is stirred for 1 h whereupon DMF is removed in vacuo. The crude is purified by silica gel P-TLC eluted with DCM/CH₃OH (10:1 or 20:1) to yield a thiol containing peptide of general structure 22. Protecting group(s) on the peptide are removed to provide additional suitable thiol containing peptides.

### Preparation of a doxorubicin-linker-peptide with alkyl linker attachment at C3′-N

To a DCM/CH₃OH (9:1) solution of 21 is added a thiol containing peptide of general structure 22 (100 mol %) prepared as described elesewhere herein. The mixture is stirred under nitrogen atmosphere in the dark for 30 min. The solvent is removed in vacuo and the resulting crude residue is dissolved into by DMSO and purified on a preparative RP-HPLC C-18 reversed phase column for purification (Method A). Fractions containing the appropriate mass, as determined by analytical HPLC-MS (Method B), were pooled and CH₃CN was removed under reduced pressure or N₂ stream followed by lyophilization to give the anthracycline-linker-peptide conjugate of general structure 23.

### g. Preparation of a anthracyclin-linker-sphingosine conjugate with linker attachment at C3′-N of doxorubicin

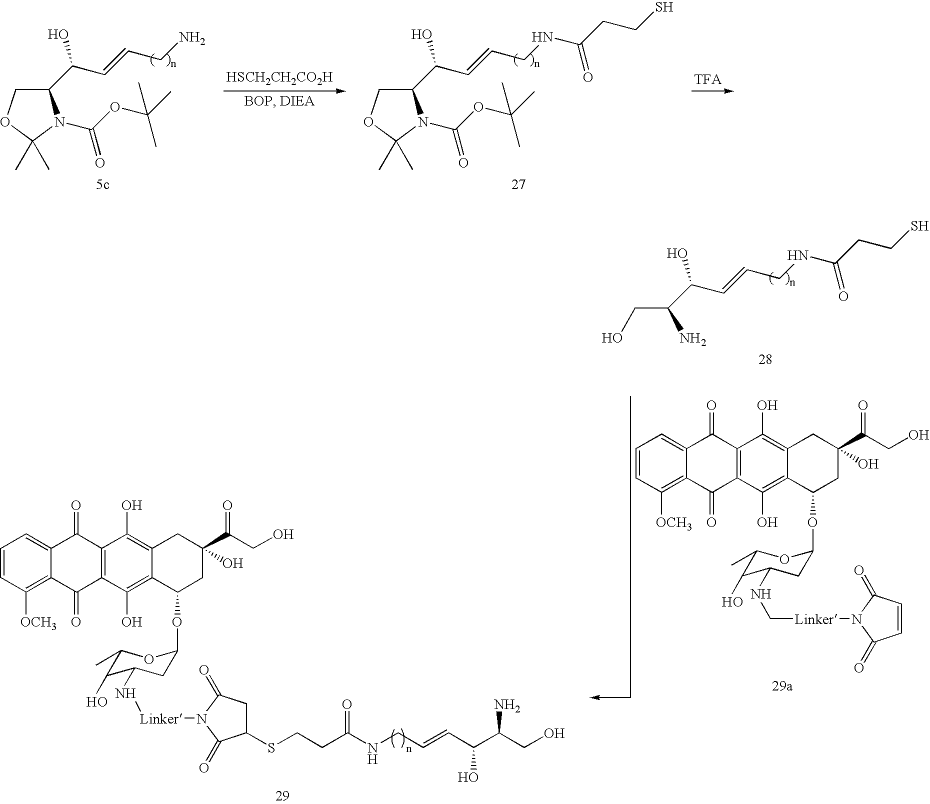

### Preparation of a thiol containing sphingosine

To head group protected (o-amino sphingosine TFA salt (5c, n=10) prepared according to the procedure of Ettmayer, P. et al., Bioorg. Med. Chem. Lett. (2004), 14:1555 in DMF is added BOP (100 mol %), DIEA (400 mol %) and HSCH₂CH₂CO₂H (100 mol %). The reaction mixture is stirred for 30 min whereupon DMF is removed in vacuo. The crude is purified by silica gel P-TLC eluted with DCM/CH₃OH (9:1) to yield the thiol containing sphingosine 27 (n=10).

### Preparation of a anthracycline-linker-sphingosine conjugate with alkyl linker attachment at C3′-N on the anthracycline

The thiol containing sphingosine 27 (n=10) is dissolved in 10% aq. TFA solution and stirred for 1 h before the solvents are evaporated. The residue (crude 28, n=10) is dissolved in MeOH/CHCl₃ (1/1) and neutralized with TEA. The maleimide doxorubicin intermediate 29a, prepared according to Example 7, is then added and the mixture is stirred in the dark for 1 h. The solvent was removed in vacuo and the resulting crude residue is dissolved into by DMSO and purified on a preparative RP-HPLC C-18 reversed phase column (Method A). Fractions containing the appropriate mass, as determined by analytical HPLC-MS (Method B), are pooled and CH₃CN is removed under reduced pressure or N₂ stream followed by lyophilization to give the anthracycline-linker-sphingosine conjugate 29 (n=10).

D. Formulation of Pharmaceutical Compositions

The pharmaceutical compositions provided herein contain therapeutically effective amounts of one or more of conjugates provided herein that are useful in the prevention, treatment, or amelioration of one or more of the symptoms of ACAMPS conditions. Such conditions include, but are not limited to, cancer, coronary restenosis, osteoporosis and syndromes characterized by chronic inflammation and/or autoimmunity. Examples of chronic inflammation and/or autoimmune diseases include but are not limited to rheumatoid arthritis and other forms of arthritis, asthma, psoriasis, inflammatory bowel disease, systemic lupus erythematosus, systemic dermatomyositis, inflammatory ophthalmic diseases, autoimmune hematologic disorders, multiple sclerosis, vasculitis, idiopathic nephrotic syndrome, transplant rejection and graft versus host disease.

The compositions contain one or more conjugates provided herein. The conjugates are formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. In one embodiment, the conjugates described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more conjugates or pharmaceutically acceptable derivatives is (are) mixed with a suitable pharmaceutical carrier or vehicle. The conjugates may be derivatized as the corresponding salts, esters, enol ethers or esters, acids, bases, solvates, hydrates or prodrugs prior to formulation, as described above. The concentrations of the conjugates in the compositions are effective for delivery of an amount, upon administration, that treats, prevents, or ameliorates one or more of the symptoms of conditions associated with ACAMPS. Such conditions include, but are not limited to, cancer, coronary restenosis, osteoporosis and syndromes characterized by chronic inflammation and/or autoimmunity. In one embodiment, the compositions are formulated for single dosage administration. To formulate a composition, the weight fraction of conjugate is dissolved, suspended, dispersed or otherwise mixed in a selected vehicle at an effective concentration such that the treated condition is relieved or ameliorated. Pharmaceutical carriers or vehicles suitable for administration of the conjugates provided herein include any such carriers known to those skilled in the art to be suitable for the particular mode of administration.

In addition, the conjugates may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a conjugate provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active conjugate is included in the pharmaceutically acceptable carrier in an amount sufficient to exert a therapeutically useful effect in the absence of undesirable side effects on the patient treated. The therapeutically effective concentration may be determined empirically by testing the conjugates in in vitro and in vivo systems described herein and then extrapolated therefrom for dosages for humans.

The concentration of active conjugate in the pharmaceutical composition will depend on absorption, inactivation and excretion rates of the active conjugate, the physicochemical characteristics of the conjugate, the dosage schedule, and amount administered as well as other factors known to those of skill in the art. For example, the amount that is delivered is sufficient to ameliorate one or more of the symptoms of diseases or disorders associated with ACAMPS condition as described herein.

In one embodiment, a therapeutically effective dosage produces a serum concentration of active ingredient of from about 0.1 ng/ml to about 50-100 μg/ml. The pharmaceutical compositions should provide a dosage of from about 0.001 mg to about 2000 mg of conjugate per kilogram of body weight per day. Pharmaceutical dosage unit forms are prepared to provide from about 1 mg to about 1000 mg and from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Pharmaceutically acceptable derivatives include acids, bases, enol ethers and esters, salts, esters, hydrates, solvates and prodrug forms. The derivative is selected such that its pharmacokinetic properties are superior to the corresponding neutral conjugate.

Thus, effective concentrations or amounts of one or more of the conjugates described herein or pharmaceutically acceptable derivatives thereof are mixed with a suitable pharmaceutical carrier or vehicle for systemic, topical or local administration to form pharmaceutical compositions. Conjugates are included in an amount effective for ameliorating one or more symptoms of, or for treating or preventing diseases or disorders associated with ACAMPS condition as described herein. The concentration of active conjugate in the composition will depend on absorption, inactivation, excretion rates of the active conjugate, the dosage schedule, amount administered, particular formulation as well as other factors known to those of skill in the art.

The compositions are intended to be administered by a suitable route, including orally, parenterally, rectally, topically and locally. For oral administration, capsules and tablets are contemplated. The compositions are in liquid, semi-liquid or solid form and are formulated in a manner suitable for each route of administration. Modes of administration include parenteral and oral modes of administration. In one embodiment, the compositions are administered orally.

Solutions or suspensions used for parenteral, intradermal, subcutaneous, or topical application can include any of the following components: a sterile diluent, such as water for injection, saline solution, fixed oil, polyethylene glycol, glycerine, propylene glycol, domethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the conjugates exhibit insufficient solubility, methods for solubilizing conjugates may be used. Such methods are known to those of skill in this art, and include, but are not limited to, using cosolvents, such as dimethylsulfoxide (DMSO), dimethylacetamide, using surfactants, such as TWEEN®, or dissolution in aqueous sodium bicarbonate.

Upon mixing or addition of the conjugate(s), the resulting mixture may be a solution, suspension, emulsion or the like. The form of the resulting mixture depends upon a number of factors, including the intended mode of administration and the solubility of the conjugate in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the conjugates or pharmaceutically acceptable derivatives thereof. The pharmaceutically therapeutically active conjugates and derivatives thereof are formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active conjugate sufficient to produce the desired therapeutic effect, in association with the required pharmaceutical carrier, vehicle or diluent. Examples of unit-dose forms include ampules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can contain along with the active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polvinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active conjugate as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active conjugate in an amount sufficient to alleviate the symptoms of the treated subject.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain 0.001%-100%, 0.1-85%, or 75-95% active ingredient.

The active conjugates or pharmaceutically acceptable derivatives may be prepared with carriers that protect the conjugate against rapid elimination from the body, such as time release formulations or coatings.

The compositions may include other active conjugates to obtain desired combinations of properties. The conjugates provided herein, or pharmaceutically acceptable derivatives thereof as described herein, may also be advantageously administered for therapeutic or prophylactic purposes together with another pharmacological agent known in the general art to be of value in treating one or more of the diseases or medical conditions referred to hereinabove, such as diseases or disorders associated with ACAMPS. It is to be understood that such combination therapy constitutes a further aspect of the compositions and methods of treatment provided herein.

1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or conjugates of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the conjugate could be provided in a composition that protects it from the acidic environment of the stomach. For example, the composition can be formulated in an enteric coating that maintains its integrity in the stomach and releases the active conjugate in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The conjugates can also be administered as a component of an elixir, suspension, syrup, wafer, sprinkle, chewing gum or the like. A syrup may contain, in addition to the active conjugates, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a conjugate or pharmaceutically acceptable derivative thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active conjugate or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603. Briefly, such formulations include, but are not limited to, those containing a conjugate provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

2. Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. Briefly, a conjugate provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The conjugate diffuses through the outer polymeric membrane in a release rate controlling step. The percentage of active conjugate contained in such parenteral compositions is highly dependent on the specific nature thereof, as well as the activity of the conjugate and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active conjugate is adjusted so that an injection provides an effective amount to produce the desired pharmacological effect. The exact dose depends on the age, weight and condition of the patient or animal as is known in the art.

The unit-dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active conjugate is an effective mode of administration. Another embodiment is a sterile aqueous or oily solution or suspension containing an active material injected as necessary to produce the desired pharmacological effect.

Injectables are designed for local and systemic administration. In some embodiments, a therapeutically effective dosage is formulated to contain a concentration of at least about 0.1% w/w up to about 90% w/w or more, or more than 1% w/w of the active conjugate to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The conjugate may be suspended in micronized or other suitable form or may be derivatized to produce a more soluble active product or to produce a prodrug. The form of the resulting mixture depends upon a number of factors, including the intended mode of administration and the solubility of the conjugate in the selected carrier or vehicle. The effective concentration is sufficient for ameliorating the symptoms of the condition and may be empirically determined.

3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a conjugate provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (10-1000 mg or 100-500 mg) or multiple dosages of the conjugate. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, about 5-35 mg or about 9-30 mg of lyophilized powder, is added per mL of sterile water or other suitable carrier. The precise amount depends upon the selected conjugate. Such amount can be empirically determined.

4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The conjugates or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of, in one embodiment, less than 50 microns, or less than 10 microns.

The conjugates may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active conjugate alone or in combination with other pharmaceutically acceptable excipients can also be administered.

These solutions, particularly those intended for ophthalmic use, may be formulated as 0.01% -10% isotonic solutions, pH about 5-7, with appropriate salts.

5. Compositions for Other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm.

Tablets and capsules for rectal administration are manufactured using the same pharmaceutically acceptable substance and by the same methods as for formulations for oral administration.

6. Articles of Manufacture

The conjugates or pharmaceutically acceptable derivatives can be packaged as articles of manufacture containing packaging material, a conjugate or pharmaceutically acceptable derivative thereof provided herein, which is used for treatment, prevention or amelioration of one or more symptoms associated with ACAMPS condition, and a label that indicates that the conjugate or pharmaceutically acceptable derivative thereof is used for treatment, prevention or amelioration of one or more symptoms associated with ACAMPS condition.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the conjugates and compositions provided herein are contemplated as are a variety of treatments for any disorder associated with ACAMPS conditions.

E. Evaluation of the Activity of the Conjugates

Standard physiological, pharmacological and biochemical procedures are available for testing the conjugates to identify those that possess biological activity, including kinase activity. In vitro and in vivo assays that can be used to evaluate biological activity, such as cytotoxicity, of the conjugates will depend upon the therapeutic agent being tested.

Exemplary assays are discussed briefly below with reference to cytotoxic conjugates (see, also, Examples). It is understood that the particular activity assayed will depend upon the conjugated therapeutic agent.

1. Protein Kinase Activity

Protein kinase activity is determined by subjecting a first end of a linker used in synthesizing linker-peptide constructs to a first test. The first test may involve observing ADP formation, an obligatory co-product of phospho group transfer from ATP which is catalyzed by the kinase to the hydroxyl group of serine, threonine or tyrosine amino acid in the peptide. Formation of ADP is followed by a coupled enzyme assay. ADP, formed from protein phosphorylation, is used by pyruvate kinase to generate pyruvate from phosphoenolpyruvate which in turn is converted to lactate by lactate dehydrogenase. The lactate results in the consumption of NADH which is followed spectrophotometrically. The rate of peptide phosphorylation is then directly related to the rate of decrease in the observed NADH signal.

Another test may involve monitoring the consumption of ATP. For example, ATP concentrations at time 0 or after 4 hour incubation may be monitored by luciferase reaction (PKLight kit obtained from Cambrex Corporation, One Meadowlands Plaza, East Rutherford, N.J. 07073), which generate a luminescence readout in the presence of ATP. Assays are initiated by mixing a kinase and a peptide in the presence of 40 μM ATP. After 4 hour of incubation at 30° C., PKLight reagent is added and mixed well, and luminescence readout measured. The rate of peptide phosphorylation is then directly related to the rate of decrease in the observed luminescence. Based on the first test, linkers of appropriate lengths and peptides with an effective amount of kinase activity which may be expected to be retained in the drug conjugate may be found.

2. Tubulin Polymerization Assay

Drug-linker constructs may further be screened using functional assays predictive of biological activity. In one example, microtubule stabilization for paclitaxel drug linker constructs or microtubule disruption by vinblastine drug-linker constructs is determined with a tubulin polymerization assay (Barron, et al., Anal. Biochem. (2003) 315:49-56). Tubulin assembly or inhibition thereof may be monitored by fluorescence using the CytoDYNAMIX Screen™ 10 kit available from Cytoskeleton (1830 S. Acoma St., Denver, Colo.). The kit is based upon an increase in quantum yield of florescence upon binding of a fluorophore to tubulin and microtubules and a 10× difference in affinity for microtubules compared to tubulin. Emission is monitored at 405 nm with excitation at 360 nm. The compounds such as paclitaxel which enhance tubulin assembly will therefore give an increase in emission whereas compounds such as vinblastine which inhibit tubulin assembly will give a decrease in emission. Tubulin assembly or inhibition may also be monitored by light scattering which is approximated by the apparent absorption at 350 nm. For paclitaxel drug conjugates BSA is employed to prevent aggregation and glycerol, which is a tubulin polymerization enhancer, is omitted from the kit to increase the signal to noise ratio.

In certain embodiments, activity of doxorubicin conjugates was assayed by monitoring alteration in the ability of Topoisomerase II to catalyze the formation of relaxed conformation DNA from a super-coiled plasmid. The more active a conjugate is at a particular concentration the less relaxed conformation DNA is produced by the action of Topoisomerase II.

In another example, a functional assay for camptothecin drug-linker constructs depends on inhibition of Topoisomerase I binding to DNA. In another example, a functional assay for camptothecin drug-linker constructs depends on inhibition of Topoisomerase I binding to DNA (Demarquay, Anti-Cancer Drugs (2001) 12:9-19).

For each type of functional assay, the enzyme (kinase) and biochemical microtubule polymerization results for all synthetic lots of each compound were combined and analyzed using GraphPad Prism® software to generate the mean±SD.

For each specific cell-based assay, results from all assays carried out with all synthetic lots of each compound were combined and analyzed using Graph Pad Prism software® to generate the mean±SD. Outliers (<7% of the total dataset) were identified and removed prior to analysis using the method of Hoaglin et al., J. Amer. Statistical Assoc., 81, 991-999, 1986. Compounds were tested between five and twenty times (in triplicate) in each assay. The significance of differences between the cytotoxic EC₅₀s of each compound against normal and tumor cell types (cytotoxic selectivity index) was determined with an unpaired t test (95% confidence interval) using GraphPad Prism® software.

Table 5 provide results for cytotoxicity, kinase activity and Topoisomerase II assay for exemplary conjugates and their parent drugs provided herein. Detailed procedures for conducting the assays are provided in the Examples section. In some embodiments, the conjugates provided herein exhibit higher cytotoxic selectivity in tumor cells as compared to their parent drugs. The conjugates are more selective for the tumor cells than the normal cells.

Tables 5, 5a and 5b provides in vitro data for the compounds whose synthesis is described in the Examples and for the parent drugs. Average EC₅₀ (“EC50-AVG”) for is provided as follows: A<0.02 μM, B=0.02-0.1 μM, C>0.1-1.0 μM and N/A=not available or inactive. Average Akt kinase activity is provided as follows: A<20, B=20-40 C>40 and N/A=not available or inactive. Average Src kinase activity is provided as follows: A<20, B=20-40 C>40 and N/A=not available or inactive. Average MPA activity is provided as follows: A<50, B=50-80, C>80 and N/A=not available or inactive. Average Tie kinase activity is provided as follows: A<20, B=20-40 C>40 and N/A=not available or inactive.

TABLE 5

| | | | MCF7 | MCF7 | HT29 | HT29 | HUVEC | HFF |
|---|---|---|---|---|---|---|---|---|
| | | Ave. | (EC50 | (EC50 | (EC50 | (EC50 | (EC50 | (EC₅₀ |
| | Ave. Akt | MPA | Ave) | Ave) | Ave) | Ave) | Ave) | Ave) |
| Systematic Name | Kinase Act. | Act. | ML | SA | ML | SA | ML | ML |
| DRUG/DRUG-AKT KINASE | | | | | | | | |
| SUBSTRATE CONJUGATE | | | | | | | | |
| Paclitaxel (PXL) | N/A | C | A | A | A | A | A | A |
| CBz-RPRTSSF-PEG(13)-10Ca-PXL | C | C | C | B | B | B | C | C |
| CBz-RPRTSSF-PEG(13)-10Ca-PXL x | C | C | C | C | C | C | C | C |
| Pv-GRPRTSSFAE(Bzl)G-PEG(13)-10Ca- | C | C | C | C | C | C | C | C |
| PXL | | | | | | | | |
| Pv-GRPRTSSFAEG-PEG(13)-10Ca-PXL | C | C | C | N/A | C | N/A | C | C |
| Pv-GRPRTSsFAEG-PEG(13)-10Ca-PXL | Zero | C | C | N/A | C | N/A | C | C |
| Pv-GRPRAAAFAEG-PEG(13)-10Ca-PXL | C | C | C | B | C | B | C | C |
| Ac-RPRTSSF-PEG(13)-10Ca-PXL | C | C | C | C | C | B | C | C |
| BOC-RPRTSSF-PEG(13)-10Ca-PXL | C | C | N/A | N/A | N/A | C | C | B |
| Ac-RSRTSSF-PEG(13)-10Ca-PXL | C | C | N/A | N/A | N/A | N/A | N/A | N/A |
| Pv-RSRKESY-PEG(13)-10Ca-PXL | C | C | N/A | N/A | N/A | N/A | N/A | N/A |
| Pv-RSRTSSFAEG-PEG(13)-10Ca-PXL | C | C | N/A | N/A | N/A | N/A | N/A | N/A |
| Pv-GRSRTSSFAEG-PEG(13)-10Ca-PXl | N/A | C | N/A | N/A | N/A | N/A | N/A | N/A |
| Vinblastine (VBL) | N/A | C | A | A | N/A | A | A | A |
| CBz-RPRTSSF-PEG(11)-3Am-VBL | C | C | A | B | A | B | A | A |
| CBz-GRPRTSSFAE(Bzl)G-3Am-VBL | C | B | B | B | B | B | A | B |
| Pv-GRPRTSSFAE(DMAB)G-3Am-VBL | C | C | C | N/A | C | N/A | C | C |
| Pv-GRPRTSSFAEG-3Am-VBL | C | C | C | N/A | C | N/A | C | C |
| CBz-RPRTSSF-PEG(29)-3Am-VBL | C | B | C | C | C | N/A | C | C |
| Ac-RPRTSSF-PEG(11)-3Am-VBL | C | C | B | N/A | B | N/A | C | B |
| BOC-RPRTSSF-PEG(11)-3Am-VBL | C | C | B | N/A | B | N/A | B | B |
| Ph(C═O)-RPRTSSF-PEG(11)-3Am-VBL | C | C | B | N/A | B | N/A | B | B |
| Ac-GRPRTSSFAEG-3Am-VBL | C | C | B | N/A | B | N/A | A | B |
| CBz-GRPRTSSFAEG-3Am-VBL | C | C | C | N/A | C | N/A | C | C |
| Pv-RSRTSSF-PEG(11)-3Am-VBL | C | C | C | N/A | C | N/A | C | C |
| CBz-RPRTSSF-PEG(11)-3Am-VBL | N/A | N/A | N/A | N/A | N/A | C | C | C |
| | Average src | | | | | | | |
| DRUG-SRC KINASE SUBSTRATES | kinase activity | | | | | | | |
| Paclitaxel (PXL) | N/A | C | A | A | A | A | A | A |
| CBz-YIYGSFK(CBz)-ALK(6)-10Es-PXL | Zero | N/A | B | N/A | C | N/A | N/A | N/A |
| H-YIYGSFK-ALK(6)-10Es-PXL | C | B | C | N/A | C | N/A | N/A | C |
| Ac-YIYGSFK-PEG(13)-10Ca-PXL | C | B | C | N/A | C | N/A | C | N/A |
| Ac-E(Bzl)YIYGSFK(CBz)-PEG(13)-10Ca- | Zero | A | N/A | N/A | N/A | N/A | N/A | N/A |
| PXL | | | | | | | | |
| Pv-YIYGSFR-PEG(13)-10Ca-PXL | C | B | C | B | C | C | C | C |
| Pv-YIYGSFR-PEG(13)-10Ca-PXL | A | B | C | N/A | C | N/A | C | C |
| Pv-YIFGSFR-PEG(13)-10Ca-PXL | Zero | A | C | B | C | C | C | C |
| Ac-EYIYGSFK-PEG(13)-10Ca-PXL | C | B | C | C | C | C | C | C |
| Ac-EYIFGSFK-PEG(13)-10Ca-PXL | Zero | B | C | N/A | C | N/A | C | N/A |
| Ac-EYIyGSFK-PEG(13)-10Ca-PXL | Zero | C | C | C | C | C | C | C |
| Ac-EYIYGSFK(CBz)-PEG(13)-10Ca-PXL | B | A | C | N/A | C | N/A | C | N/A |
| Pv-E(Bzl)YIYGSFK(CBz)-PEG(13)-10Ca- | A | A | C | B | C | C | C | C |
| PXL | | | | | | | | |
| Pv-EYIYGSFR-PEG(13)-10Ca-PXL | B | B | C | C | C | C | C | C |
| Ac-YIYGSFR-PEG(13)-10Ca-PXL | C | B | C | N/A | C | N/A | C | C |
| Ac-EYIYGSFR-PEG(13)-10Ca-PXL | B | B | C | N/A | C | N/A | C | C |
| Pv-YIYGSFR-PEG(13)-10Ca-PXL | N/A | B | C | B | C | C | C | C |
| H-YIYGSFK-PEG(11)-3Am-VBL | C | B | C | N/A | C | N/A | C | B |
| BOC-YIYGSFK(BOC)-PEG(11)-3Am-VBL | A | B | B | N/A | B | N/A | C | C |
| BOC-YIYGSFK(BOC)-PEG(29)-3Am-VBL | | | | | | | | |
| H-YIYGSFK-PEG(29)-3Am-VBL x 2TFA | A | A | A | N/A | B | N/A | C | B |
| BOC-YI(phospho)YGSFK(BOC)-PEG(11)- | C | C | C | N/A | N/A | N/A | C | B |
| 3Am-VBL | | | | | | | | |
| H-YI(phospho)YGSFK-PEG(11)-3Am-VBL | N/A | A | C | N/A | C | N/A | N/A | N/A |
| CBz-YIYGSFK(BOC)-PEG(11)-3Am- | A | C | C | N/A | C | N/A | C | N/A |
| VBL | | | | | | | | |
| CBz-YIYGSFK-PEG(11)-3Am-VBL | C | C | C | C | C | C | C | C |
| CBz-YIFGSFK-PEG(11)-3Am-VBL | A | B | C | N/A | C | N/A | C | C |
| CBz-YIYGSFK-PEG(11)-3Am-VBL | A | B | C | C | C | C | C | C |
| Ac-YIFGSFK(BOC)-PEG(11)-3Am-VBL | B | B | C | N/A | C | N/A | C | C |
| Ac-YIFGSFK-PEG(11)-3Am-VBL | C | B | C | N/A | C | N/A | C | C |
| CBz-E(Bzl)YIFGSFK(BOC)-PEG(11)-3Am- | A | A | A | N/A | C | N/A | C | C |
| VBL | | | | | | | | |
| CBz-E(Bzl)YIFGSFK-PEG(11)-3Am-VBL | C | B | C | C | C | C | C | C |
| Ac-YIYGSFR-PEG(11)-3Am-VBL | C | B | B | B | B | C | C | C |
| Pv-YIYGSFR-PEG(11)-3Am-VBL | B | C | C | C | C | C | C | C |
| BOC-EYIYGSFK(BOC)-PEG(11)-3Am-VBL | B | C | C | C | C | C | C | C |
| Pv-E(DMAB)YIYGSFR-PEG(11)-3Am-VBL | A | B | B | B | C | B | B | C |
| Pv-EYIYGSFR-PEG(11)-3Am-VBL | C | C | C | N/A | C | N/A | C | C |
| BOC-YIYGSFR-PEG(11)-3Am-VBL | B | C | C | C | C | C | C | C |
| BOC-E(Bzl)YIFGSFK(BOC)-PEG(11)-3Am- | A | A | B | A | C | B | C | C |
| VBL | | | | | | | | |
| CBz-YIYGSFK(CBz)-PEG(11)-3Am-VBL | A | B | B | N/A | C | N/A | B | B |
| BOC-YIYGSFS-PEG(11)-3Am-VBL | C | C | C | N/A | C | N/A | C | C |
| Ac-EYIYGSFR-PEG(11)-3Am-VBL | B | C | C | C | C | C | C | C |
| CH3O(CH2CH2O)3CH2CH2(C═O)YIYGSFS- | C | C | C | N/A | C | N/A | C | C |
| PEG(11)-3Am-VBL | | | | | | | | |
| Ac-YIYGSFS-PEG(11)-3Am-VBL | C | B | C | N/A | C | N/A | C | C |
| Ac-YIYGSFH-PEG(11)-3Am-VBL | C | C | C | N/A | C | N/A | C | C |
| CH3O(CH2CH2O)3CH2CH2(C═)YIYGSF | C | B | C | N/A | C | N/A | C | C |
| H-PEG(11)-3Am-VBL | | | | | | | | |
| CBz-GIYWHHY-PEG(11)-3Am-VBL | A | B | C | N/A | C | N/A | N/A | N/A |
| BOC-GIYWHHY-PEG(11)-3Am-VBL | A | B | C | N/A | C | N/A | N/A | N/A |
| H-GIYWHHY-PEG(11)-3Am-VBL | B | B | C | N/A | C | N/A | C | C |
| BOC-GIYWHHY-PEG(29)-3Am-VBL | A | B | C | N/A | N/A | N/A | C | C |
| H-GIYWHHY-PEG(29)-3Am-VBL | C | C | C | N/A | N/A | N/A | C | C |
| | | TOPO | | | | | | |
| | Src (Qual. | (% | | | | | | |
| DOX-SRC KINASE SUBSTRATE | Act.) | activity | | | | | | |
| H-YIYGSFK-3′Am-MAL(8)-DOX | C | A | C | N/A | C | N/A | C | N/A |
| H-YIYGSFK-3′Alk-MAL(9)-DOX | C | C | C | N/A | N/A | N/A | N/A | C |
| CBz-YIYGSFK-3′Alk-MAL(9)-DOX | C | A | C | N/A | N/A | N/A | C | N/A |
| Ac-YIYGSFK-3′Alk-MAL(9)-DOX | C | N/A | C | N/A | N/A | N/A | N/A | N/A |
| Ac-EYIYGSFK-3′Alk-MAL(9)-DOX | C | N/A | C | N/A | N/A | N/A | C | C |
| Vinblastine (VBL)-TIE KINASE | Tie2 kinase | | | | | | | |
| SUBSTRATE | activity | | | | | | | |
| Vinblastine (VBL) | 0 | C | A | A | A | A | A | A |
| Pv-RLVAYE(Bzl)GYV-PEG(11)-3Am- | N/A | A | B | N/A | C | N/A | C | N/A |
| VBL | | | | | | | | |
| Pv-RLVAYE(DMAB)GYV-PEG(11)- | N/A | A | C | N/A | C | N/A | C | C |
| 3Am-VBL | | | | | | | | |
| Pv-RLVAYEGYV-PEG(11)-3Am-VBL | N/A | B | C | N/A | C | N/A | C | C |
| Pv-RLVAYE(Bzl)GYV-PEG(13)-10Ca- | N/A | A | C | N/A | C | N/A | C | N/A |
| PXL | | | | | | | | |
| Pv-RLVAYEGYV-PEG(13)-10Ca-PXL | N/A | A | C | N/A | C | N/A | C | C |

TABLE 5a
PACLITAXEL NON-TARGETED DERIVATIVES

| | Ave. | | MCF7 | MCF7 | HT29 | HT29 | HUVEC | HFF |
|---|---|---|---|---|---|---|---|---|
| | TK1 | Ave. | (EC50 | (EC50 | (EC50 | (EC50 | (EC50 | (EC50 |
| | Kinase | MPA | Ave) | Ave) | Ave) | Ave) | Ave) | Ave) |
| Systematic Name | Act. | Act. | ML | SA | ML | SA | ML | ML |
| Paclitaxel (PXL) | A | C | A | A | A | A | A | A |
| PXL-7Es-ALK(5)-NH2 | — | A | C | A | C | A | A | A |
| PXL-7Ca-ALK(6)-NH2 | — | A | C | A | A | A | A | a |
| PXL-7Ca-ALK(6)-Phospho(OPh, | — | A | B | A | A | A | A | A |
| N-Ala) | | | | | | | | |
| PXL-7Ca-ALK(6)-diphenyl | — | A | B | A | A | A | A | A |
| phosphoramidate | | | | | | | | |
| PXL-2′Alloc | — | A | A | A | A | A | A | A |
| PXL-10Es-Alk(6)-NH(Z) | — | A | A | A | A | A | A | A |
| 10 Deacetyl Taxol | — | — | B | A | A | A | A | A |
| PXL-10Es-ALK(5)-NH2 | — | B | A | A | A | A | A | A |
| PXL-10Ca-PEG(13)-NH(Z) | — | B | A | A | A | A | A | A |

TABLE 5b
VINBLASTINE NON-TARGETED DERIVATIVES

| | Ave. | | MCF7 | MCF7 | HT29 | HT29 | HUVEC | HFF |
|---|---|---|---|---|---|---|---|---|
| | Tie2 | Ave. | (EC50 | (EC50 | (EC50 | (EC50 | (EC50 | (EC50 |
| | Kinase | MPA | Ave) | Ave) | Ave) | Ave) | Ave) | Ave) |
| Systematic Name | Act. | Act. | ML | SA | ML | SA | ML | ML |
| Vinblastine (VBL) | — | C | A | A | A | A | A | A |
| VBL-3Am-ALK(8)-NH2 | — | B | A | A | A | A | A | A |
| VBL-3Am-ALK(6)-NH(B) | — | A | A | A | A | A | A | A |
| VBL-3Am-ALK(6)-NH2 | — | C | A | A | A | A | A | A |
| VBL-3Am-ALK(12)-NH(B) | — | A | B | A | C | A | A | A |
| VBL-3Am-ALK(12)-NH2 | — | B | A | A | A | A | A | A |
| VBL-3Am-PEG(11)-NH(B) | — | B | A | A | A | A | A | A |
| VBL-3Am-PEG(11)-NH2 | — | B | B | A | B | A | A | A |
| Desacetylvinblastine monohydrazine | — | C | A | A | A | A | A | A |
| Desacetyl vinblastine | — | C | A | A | A | A | A | A |

In certain embodiments, as demonstrated by a comparison of the cytotoxic selectivity for exemplary conjugates and parent drugs in tumors and normal cells, the conjugates show increase in the cytotoxic selectivity for tumor cells as compared to the cytotoxic selectivity of the parent drug:

| | | | HT-29 |
|---|---|---|---|
| | HFFMonolayer | MCF-7 Soft Agar | Soft Agar |
| Drug/Conjugate | EC50 (nM) | EC50 (nM) | EC50 (nM) |
| Paclitaxel | 9 ± 5 | 6 ± 3 | 15 ± 2 |
| Pv-YIYGSFR- | 2,139 ± 873 | 80 ± 13 | 175 ± 43 |
| PEG(13)-10Ca-PXL | | | |
| Ac- | 4,731 ± 3406 | 197 ± 76 | 231 ± 30 |
| EYIYGSF- | | | |
| PEG(13)-10Ca-PXL | | | |
| Vinblastine | 1.5 ± 0.5 | 7 ± 5 | 7 ± 7 |
| CBz-YIYGSFK- | 655 ± 186 | 156 ± 37 | 464 ± 351 |
| PEG(11)-3Am-VBL | | | |

The improvement in the cytotoxic selectivity of exemplary conjugates as compared to the cytotoxic selectivity of paclitaxel and vinblastine in exemplary cell lines, as illustrated by improved cytotoxic selectivity index, is shown below:

Cytotoxic Selectivity Index

| Drug/conjugate | HFF/MCF7 | HFF/HT29 |
|---|---|---|
| PXL | 1.4 | 0.6 |
| Pv-YIYGSFR- | 26.6 | 12.3 |
| PEG(13)-10Ca-PXL | | |
| Ac-EYIYGSFK- | 24.1 | 20.5 |
| PEG(13)-10Ca-PXL | | |
| Vinblastine | 0.2 | 0.2 |
| CBz-YIYGSFK- | 4.2 | 1.4 |
| PEG(11)-3Am-VBL | | |

In certain embodiments, the conjugates show better serum stability as compared to the parent drug as demonstrated by an exemplary conjugate below:

| | Initial | Relative Percent Remaining at | | | | | |
|---|---|---|---|---|---|---|---|
| Drug/conjugate | Concentration(μM) | 0 hr | 4 hr | 8 hr | 24 hr | 72 hr | T½ hr |
| Paclitaxel | 8.9 | 100 | 73 | 59 | 28 | <3.0 | 11 |
| Pv-YIYGSFR- | 9.4 | 100 | 73 | 63 | 63 | 64 | >72 |
| PEG(13)-10Ca- | | | | | | | |
| PXL | | | | | | | |
| Ac-EYIYGSFK- | 12 | 100 | 95 | 99 | 69 | 22 | 32 |
| PEG(13)-10Ca- | | | | | | | |
| PXL | | | | | | | |
| Vinblastine | 12 | 100 | 95 | 95 | 87 | 67 | >72 |
| CBz-YIYGSFK- | 8.4 | 100 | 88 | 102 | 100 | 61 | >72 |
| PEG(11)-3Am- | | | | | | | |
| VBL | | | | | | | |

One skilled in the art will appreciate that the assays described here may also be used to screen for direct substrate-drug conjugates (i.e., conjugates which contain no linker).

F. Methods of use of the Conjugates and Compositions

Methods of use of the conjugates and compositions provided herein are also provided. The methods involve both in vitro and in vivo uses of the conjugates and compositions. The methods provided herein can be used for increasing drug efficiency. In certain embodiments, methods for treating conditions caused by undesirable chronic or aberrant cellular activation, migration, proliferation or survival (ACAMPS) are provided.

ACAMPS conditions are characterized by undesirable or aberrant activation, migration, proliferation or survival of tumor cells, endothelial cells, B cells, T cells, macrophages, granulocytes including neutrophils, eosinophils and basophils, monocytes, platelets, fibroblasts, other connective tissue cells, osteoblasts, osteoclasts and progenitors of many of these cell types. Examples of ACAMPS-related conditions include, but are not limited to, cancer, coronary restenosis, osteoporosis and syndromes characterized by chronic inflammation and/or autoimmunity. Examples of chronic inflammation and/or autoimmune diseases include but are not limited to rheumatoid arthritis and other forms of arthritis, asthma, psoriasis, inflammatory bowel disease, systemic lupus erythematosus, systemic dermatomyositis, inflammatory ophthalmic diseases, autoimmune hematologic disorders, multiple sclerosis, vasculitis, idiopathic nephrotic syndrome, transplant rejection and graft versus host disease.

Examples of cancers include, but are not limited to, non-small cell lung cancer, small cell lung cancer, head and neck squamous cancers, colorectal cancer, prostate cancer, and breast cancer, acute lymphocytic leukemia, adult acute myeloid leukemia, adult non-Hodgkin's lymphoma, brain tumors, cervical cancers, childhood cancers, childhood sarcoma, chronic lymphocytic leukemia, chronic myeloid leukemia, esophageal cancer, hairy cell leukemia, kidney cancer, liver cancer, multiple myeloma, neuroblastoma, oral cancer, pancreatic cancer, primary central nervous system lymphoma, skin cancer, and small-cell lung cancer. Childhood cancers amenable to treatment by the methods and with the compositions provided herein include, but are not limited to, brain stem glioma, cerebellar astrocytoma, cerebral astrocytoma, ependymoma, Ewing's sarcoma and family of tumors, germ cell tumor, Hodgkin's disease, ALL, AML, liver cancer, medulloblastoma, neuroblastoma, non-Hodgkin's lymphoma, osteosarcoma, malignant fibrous histiocytoma of bone, retinoblastoma, rhabdomyosarcoma, soft tissue sarcoma, supratentorial primitive neuroectodermal and pineal tumors, unusual childhood cancers, visual pathway and hypothalamic glioma, Wilms' tumor, and other childhood kidney tumors.

The methods and compositions provided can also be used to treat cancers that originated from or have metastasized to the bone, brain, breast, digestive and gastrointestinal systems, endocrine system, blood, lung, respiratory system, thorax, musculoskeletal system, and skin. The methods are generally applicable to all cancers but have particularly significant therapeutic benefit in the treatment of solid tumors. In certain embodiments, the solid tumors are characterized by extensive regions of hypoxic tissue. In certain embodiments, the drug moieties provided in Table 4 are used in the conjugates, which are used in treating particular types of cancer.

Table 3 provides examples of enzymes that are overexpressed or activated in primary disease tissue of a malignant phenotype. The use of substrates for such enzymes wherein the action of the enzyme on the substrate results in entrapment of the drug-substrate allows for selective trapping of drugs in the tumor cells. Table 4 provides examples of drug moieties for use in the conjugates provided herein, which are used in treating particular types of cancer.

TABLE 3
Trapping Target Selection for Cancer

| Enzyme | Pathway | Aberrant Expression/Activity |
|---|---|---|
| Akd Cytoplasmic Ser/Thr Kinase | Apoptosis | Essentially all tumors |
| Src Cytoplasmic Tyrosine Kinase | Proliferation | Breast, Lung, Colorectal, etc. |
| VEGF Receptor Tyrosine Kinase | Angiogenesis | All tumor vasculature |
| Tie-2 Receptor Tyrosine Kinase | Angiogenesis | All tumor vasculature |
| c-Met Receptor Tyrosine Kinase | Proliferation | Glioma, Colorectal, Pancreatic, |
| | | Melanoma |
| Abl Tyrosine Kinase | Proliferation | Leukemia |
| EGF Receptor Tyrosine Kinase | Proliferation | Many solid tumors |
| PDGF Receptor Tyrosine Kinase | Proliferation | Many solid tumors |
| Raf Serine/Threonine Kinase | Proliferation | Ras Pathway in many solid tumors |

TABLE 4
Drug Selection

Paclitaxel (Taxane)
Breast, Lung, Prostate, Ovarian, Head & Neck, Esophageal, Bladder
Doxorubicin (Anthracycline)
Breast, Lung, Ovarian, Bladder, Hepatoma, Neuroblastoma, Lymphoma
Vinblastine (Vinca Alkaloid)
Breast, Lung, Prostate, Testicular, Renal, Lymphoma
Methotrexate (Antimetabolite)
Breast, Colorectal, Head & Neck, Leukemia, Lymphoma
Cisplatin (DNA Crosslinking Agent)
Lung, Ovarian, Head & Neck, Esophageal, Bladder, Lymphoma

G. Library and Screening Methods

The conjugates provided herein can be produced using combinatorial methods to produce large libraries of potential conjugates. Methods for producing and screening combinatorial libraries of molecules are known in the art. The libraries of potential conjugates can then be screened for identification of a conjugate with the desired characteristics. Any convenient screening assay can be employed, where the particular screening assay may be known to those of skill in the art or developed in view of the specific molecule and property being studied.

For example, the libraries of potential conjugates can be screened for selectivity by comparing the conjugate activity in the target cell or tissue type to conjugate activity in cells or tissues in which drug activity is not desired. A selective conjugate will affect the target in the desired cells (e.g., cells involved in a disease process), but affect the target in undesired cells to a lesser extent or not at all. In another example, the libraries of potential conjugates can be screened for conjugates that exhibit enhanced drug efficiency as compared to the pharmacological activity of the unconjugated drug. For example, a more efficient drug will result in a desirable pharmacological response at a lower effective dose than a less efficient drug. In another example, a more efficient drug will have an improved therapeutic index compared to a less efficient drug. In one example, the screening assay will involve observing the accumulation of the conjugate in the target system, in comparison to that of the unconjugated drug.

H. High throughput Screening and Target Identification Methods for Kinase Substrate Trapping Sequences using Drug-Linker-Peptide Conjugate Libraries

The methods provided herein are generally applicable peptide properties and methods to make drug-linker-peptide conjugates that retain drug and peptide substrate activity, as well as cell permeability. Peptide libraries 3 to 20 amino acids in length can be produced using phage or solid phase techniques by someone skilled in the art, using published methods. Drugs such as paclitaxel and vinblastine can be prepared with a biotin moiety or fluorescent tag using procedures known in the art. (See, e.g., Guillemard et al., Anticancer Res. November-December 1999; 19(6B):5127-30; Dubois et al., Bioorg Med Chem. October 1995; 3(10):1357-68; Chatterjee et al., Biochemistry. Nov. 26, 2002; 41(47):14010-8; Baloglu et al., Bioorg Med Chem Lett. Sep. 3, 2001; 11(17):2249-52; Li et al., Biochemistry. Jan. 25, 2000; 39(3):616-23; Rao et al., Bioorg Med Chem. November 1998; 6(11):2193-204; Bicamumpaka et al., Int J Mol Med. August 1998; 2(2):161-165; Sengupta et al., Biochemistry. Apr. 29, 1997; 36(17):5179-84; Han et al., Biochemistry. Nov. 12, 1996; 35(45):14173-83; Sengupta et al., Biochemistry. Sep. 19, 1995; 34(37):11889-94).

For example, peptide libraries can be conjugated to drugs (such as paclitaxel or vinblastine) which contain a biotin moiety or a fluorescent tag. A fluorescent drug (such as doxorubicin can also be used). In the case of biotinylated conjugates, the libraries need not be purified. Large mixtures of compounds can be incubated with various target cells (ACAMPS disease or normal), followed by removal of the extracellular medium, cell washing and isolation of phosphorylated (trapped) conjugates from cell lysates using streptavidin or avidin affinity chromatography. Determination of the sequence of the trapped peptide by standard methods will identify a substrate of an overexpressed or activated kinase expressed in the diseased cell type (or disease-associated normal cell type). This provides a trapping substrate candidate, which can then be used with the original drug or linked to other drugs and optimized.

Fluorescently tagged conjugates can be used with drug-peptide conjugate libraries that are produced in a “one compound per well” format. The libraries are incubated with tumor cells, endothelial cells or cells derived from any (ACAMPS) disease tissue, in a multi-well format, followed by washing and determination of well-associated fluorescence. Fluorescent drug-peptide conjugates that are retained to a high extent by diseased or other target cells represent novel drug candidates. Additionally, specificity can be assessed by comparing fluorescence uptake in the target cell to that in a normal cell type or one not associated with the disease of interest. The above methods are not limited to biotinylated or fluorescently tagged conjugates, but can be carried out with any tag or inherent property that facilitates purification or spectrophotometric visualization of conjugates specifically trapped or accumulated in target cells.

Since consensus substrate sequences are known for a large number of kinases, it is also possible to use these methods to identify new drug discovery (enzyme inhibition) targets for any ACAMPS disease. In other words, the methods can be used to identify an overexpressed or aberrantly activated kinase that has not previously been associated with a particular disease. In the instances where a biotinylated drug-substrate conjugate is employed, it could also be used to isolate the kinase in question from cell extracts via affinity chromatography. The kinase may be a previously identified or novel enzyme. The library and screening methods and novel approaches described above may also be applied to small molecule or metabolic kinase substrates.

G. Combination Therapy

The conjugates provided herein may be administered as the sole active ingredient or in combination with other active ingredients. Other active ingredients that may be used in combination with the conjugates provided herein include but are not limited to, compounds known to treat ACAMPS conditions, anti-angiogenesis agents, anti-tumor agents, other cancer treatments and autoimmune agents. Such compounds include, in general, but are not limited to, alkylating agents, toxins, antiproliferative agents and tubulin binding agents. Classes of cytotoxic agents for use herein include, for example, the anthracycline family of drugs, the vinca drugs, the mitomycins, the bleomycins, the cytotoxic nucleosides, the pteridine family of drugs, diynenes, the maytansinoids, the epothilones, the taxanes and the podophyllotoxins.

It is understood that the foregoing detailed description and accompanying examples are merely illustrative, and are not to be taken as limitations upon the scope of the subject matter. Various changes and modifications to the disclosed embodiments will be apparent to those skilled in the art. Such changes and modifications, including without limitation those relating to the chemical structures, substituents, derivatives, intermediates, syntheses, formulations and/or methods of use provided herein, may be made without departing from the spirit and scope thereof. U.S. patents and publications referenced herein are incorporated by reference.

### EXAMPLES

Abbreviations used: Boc, t-butyloxycarbonyl; BOP, benzotriazol-1-yloxytris-(dimethylamino)phosphonium hexafluorophosphate; Cbz, benzyloxycarbonyl; CDI, 1,1′-carbonyldiimidazole; DCC, 1,3-dicyclohexylcarbodiimide; DCM, dichloromethane; DIEA, N,N-diisopropylethylamine; DIPC, 2-dipyridylcarbonate; DMAP, 4-(dimethylamino)pyridine; DMF, N,N-dimethylformamide; DMSO, dimethylsulfoxyde; MS, mass spectroscopy; RP-HPLC, reversed phase high performance liquid chromatography; TFA, trifluoroacetic acid; THF, tetrahydrofuran; RT, room temperature. Preparative RP-HPLC purification was conducted on YMC-Pack ODS-A columns (S-5 μM, 300×20 mm ID) with gradient elution between 0% B to 50% B or 0% B to 100% B (A=0.1% TFA in H₂O; B=0.1% TFA in CH₃CN) with gradient times of 10 min and a flow rate of 25 mL/min with UV 220 nm detection (Method A). Analytical HPLC-MS was conducted on a YMC Combi-Screen ODS-A column (S-5 μM, 50×4.6 mm ID) with gradient elution of %0 B to 100% B (A=0.1% TFA in H₂O; B=0.1% TFA in CH₃CN) with gradient times of 10 min and a flow rate of 3.5 mL/min with UV 220 nm and Electrospray MS detection (Method B).

### Example 1

### Peptide Synthesis Procedures

General Procedure

Peptide synthesis was conducted on an Applied Biosystems (ABI, Foster City, Calif., USA) model 433A synthesizer using solid-phase FastMoc™ chemistry programmed with SynthAssist software V.2.0.2 provided by the manufacturer. In FastMoc™, the amino acids are activated with HBTU (2-(1H-benzotriazol-1-yl) 1,1,3,3-tetramethyluronium hexaflurophosphate) and DIEA is used as base. Preloaded resins, amino acids and reagents were purchased from Bachem, Peptide International, Senn Chemicals, Novabiochem and Advanced Chemtech.

All peptides were prepared following general procedure A, B or C. Some of the representative examples are given here.

General Procedure A. On a typical 0.25 mmol scale synthesis on Wang resin (4-alkoxybenzyl alcohol resin), the peptide was cleaved with 1.5 h shaking using 10 mL of a 94% trifluoroacetic acid, 3%p-cresol and 3% triisopropylsilane v/v mixture. An extra 5% H₂O was required for peptides containing pbf (2,2,4,6,7-pentamethyldihydrobenzofuran-5-sulfonyl) side chain protecting group on Arginine. The cleavage mixture was filtered through polypropylene cartridge with a polyethylene hydrophobic frit. The supernatant was concentrated by evaporation to half the volume and then added to 50 mL ice-cold ethyl ether. Peptide precipitate was collected, dried in vacuo, dissolved in DMSO and purified by Preparative RP-HPLC (Method A). Fractions containing the appropriate mass, as determined by analytical HPLC-MS (Method B) were pooled and CH₃CN was removed with a stream of N₂. The remaining aqueous mixture was then lyophilized obtaining the desired peptide.

General Procedure B. On a typical 0.25 mmol scale synthesis on Wang resin (4-alkoxybenzyl alcohol resin), the peptide was cleaved with 1.5 h shaking using 10 mL of a 94% trifluoroacetic acid, 3%p-cresol and 3% triisopropylsilane v/v mixture. An extra 5% H₂O was required for peptides containing pbf (2,2,4,6,7-pentamethyldihydrobenzofuran-5-sulfonyl) side chain protecting group on Arginine. The cleavage mixture was filtered through polypropylene cartridge with a polyethylene hydrophobic frit. The supernatant was concentrated by evaporation to half the volume and then added to 50 mL ice-cold ethyl ether. Peptide precipitate was collected, dissolved in CH₃CN/H₂O and lyophilized. This is to hydrolyze possible TFA adducts formed on the side chain hydroxyl groups. The crude peptide was then dissolved in DMSO and purified by Preparative RP-HPLC (Method A). Fractions containing the appropriate mass, as determined by analytical HPLC-MS (Method B) were pooled and CH₃CN was removed with a stream of N₂. The remaining aqueous mixture was then lyophilized obtaining the desired peptide.

General Procedure C. On a typical 0.25 mmol scale synthesis on 2-Cl-trityl resin, the peptide was cleaved using ca. 60-70 mL 1% TFA in CH₂Cl₂ in several portions each with 2-5 min shaking. Pyridine was added to neutralize the solution and the solvents were evaporated. The crude peptide and pyridinum salt were then dissolved in DMSO and purified by Preparative RP-HPLC (Method A). Fractions containing the appropriate mass, as determined by analytical HPLC-MS (Method B) were pooled and CH₃CN was removed with a stream of N₂. The remaining aqueous mixture was then lyophilized obtaining the desired peptide.

Synthesis of exemplary peptides used in the conjugates provided herein is descrined:

1) Preparation of Pv-YIYGSFR—OH

Synthesis was conducted on ABI 433A using general procedure A with the following resin (0.25 mmol) and Fmoc-amino acids (1.1 mmol, 4.4 mol equiv.) as well as trimethylacetic acid (pivalic acid or PvOH) (1.1 mmol, 4.4 mol equiv.) as the capping group:
Fmoc-Arg(pbf)-Wang resin
Fmoc-Phe-OH
Fmoc-Ser(OtBu)-OH
Fmoc-Gly-OH
Fmoc-Tyr(OtBu)-OH
Fmoc-Ile-OH
Fmoc-Tyr(OtBu)-OH
PvOH

RP-HPLC purification gave an average 150 mg desired peptide (>95% purity, 60.7% yield). Electrospray (LCMS) m/z 990 (M+H⁺, C₄₉H₆₈N₁₀O₁₂ requires 990); retention time=4.23 min (1% to 99% B, Method B).

2) Preparation of E(bzl)Src2(Ac,Z) or Ac-E(OBz1)YIYGSFK(Z)-OH

Synthesis was conducted on ABI 433A using general procedure A with the following resin (0.25 mmol) and Fmoc-amino acids (1.1 mmol, 4.4 mol equiv.) as well as acetic acid (AcOH) (1.1 mmol, 4.4 mol equiv.) as the capping group:
Fmoc-Lys(Z)-Wang resin
Fmoc-Phe-OH
Fmoc-Ser(OtBu)-OH
Fmoc-Gly-OH
Fmoc-Tyr(OtBu)-OH
Fmoc-Ile-OH
Fmoc-Tyr(OtBu)-OH
Fmoc-Glu(OBz1)-OH
AcOH

RP-HPLC purification gave an average 85 mg desired peptide ((>95% purity, 26.7% yield). Electrospray (LCMS) m/z 1273 (M+H⁺, C₆₆H₈₁N₉O₁₇ requires 1273); retention time=5.68 min (1% to 99% B, Method B).

3) Preparation of Src2(Z,B) or Z-YIYGSFK(B)-OH

Synthesis was conducted on ABI 433A using general procedure C with the following resin (0.25 mmol), Fmoc-amino acids (1.1 mmol, 4.4 mol equiv.) and Z-Tyr-OH (1.1 mmol, 4.4 mol equiv.) as the N-terminal residue.
H-Lys(Boc)-2-Cl-trityl resin
Fmoc-Phe-OH
Fmoc-Ser(Trt)-OH
Fmoc-Gly-OH
Fmoc-Tyr(2-ClTrt)-OH
Fmoc-Ile-OH
Z-Tyr-OH

RP-HPLC purification gave an average 162 mg desired peptide ((>95% purity, 58.4% yield). Electrospray (LCMS) m/z 1112 (M+H⁺, C₅₇H₇₄N₈O₁₅ requires 1112); retention time=5.62 min (1% to 99% B, Method B).

4) Preparation of Akt1 (Pv, Bz1) or Pv-GRPRTSSFAE(OBz1)G-OH

Synthesis was conducted on ABI 433A using general procedure B with the following resin (0.25 mmol) and Fmoc-amino acids (1.1 mmol, 4.4 mol equiv.) as well as tritethylacetic acid (pivalic acid or PvOH) (1.1 mmol, 4.4 mol equiv.) as the capping group:
Fmoc-Gly-Wang resin
Fmoc-Glu(OBz1)-OH
Fmoc-Ala-OH
Fmoc-Phe-OH
Fmoc-Ser(OtBu)-OH
Fmoc-Ser(OtBu)-OH
Fmoc-Thr(OtBu)-OH
Fmoc-Arg(pbf)-OH
Fmoc-Pro-OH
Fmoc-Arg(pbf)-OH
Fmoc-Gly-OH
PvOH

RP-HPLC purification gave 87 mg desired peptide ((>95% purity, 26.0% yield). Electrospray (LCMS) m/z 1339 (M+H⁺, C₆₀H₉₁N₁₇O₁₈ requires 1339); retention time=3.76 min (1% to 99% B, Method B).

5) Preparation of Akt (Pv,dmab) or Pv-GRPRTSSFAE(Odmab)G-OH

Synthesis was conducted on ABI 433A using general procedure B with the following resin (0.25 mmol) and Fmoc-amino acids (1.1 mmol, 4.4 mol equiv.) as well as trimethylacetic acid (pivalic acid or PvOH) (1.1 mmol, 4.4 mol equiv.) as the capping group.
Fmoc-Gly-Wang resin
Fmoc-Glu(ODmab)-OH
Fmoc-Ala-OH
Fmoc-Phe-OH
Fmoc-Ser(OtBu)-OH
Fmoc-Ser(OtBu)-OH
Fmoc-Thr(OtBu)-OH
Fmoc-Arg(pbf)-OH
Fmoc-Pro-OH
Fmoc-Arg(pbf)-OH
Fmoc-Gly-OH
PvOH

RP-HPLC purification gave 49 mg desired peptide (90% purity, 12.6% yield). Electrospray (LCMS) m/z 1561 (M+H⁺, C₇₃H₁₁₀N₁₈O₂₀ requires 1561); retention time=4.51 min (1% to 99% B, Method B).

### Example 2

### i). Preparation of 2′-O-(tert-butyldimethylsilyl)-7-O-(triethylsilyl)-10-deacetyl-10-O-(carbonylimidazolyl)paclitaxel (6a)

To 2′-O-(tert-butyldimethylsilyl)-7-O-(triethylsilyl)-10-deacetyl-paclitaxel (5a, 845 mg, 0.81 mmol), prepared according to the procedure in Datta, A.; Hepperle, M. I. G. J. Org. Chem. (1995) 60:761, in anhydrous DCM (6 mL) was added carbonyldiimidazole (530 mg, 400 mol %). The reaction mixture was allowed to stir for 16 hours at room temperature under nitrogen atmosphere then extracted with water (5 mL). The organic layer was dried over sodium sulfate, filtered and concentrated to give 890 mg of the title compound 6a which was subsequently used without purification.

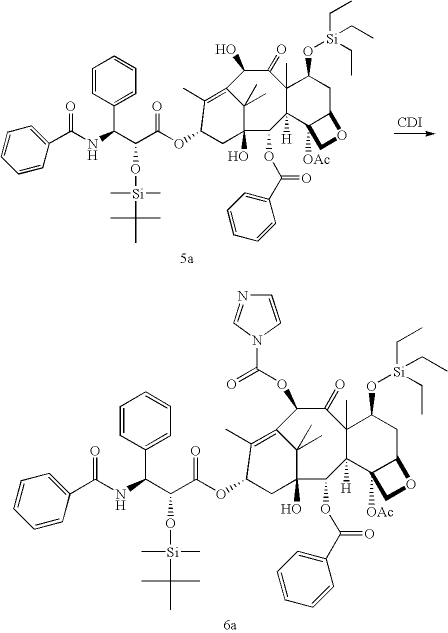

### ii). Preparation of paclitaxel-10-(deacetyl)-10-O-(carbamoyl-PEG-amine) (32)

### Step A: Reaction of 2′-O-(tert-butyldimethylsilyl)-7-O-(triethylsilyl)-10-deacetyl-10-O-(carbonylimidazolyl)paclitaxel (6a) with benzyl-3-[2-[2-[3-aminopropoxy]-ethoxy]-ethoxy]-propylcarbonate (31)

To 2′-O-(tert-butyldimethylsilyl)-7-O-(triethylsilyl)-10-O-deacetyl-10-O-(carbonylimidazolyl)paclitaxel (6a, 250 mg, 0.22 mmol), prepared as described above, dissolved in anhydrous tert-butyl alcohol (5 mL) was benzyl-3-[2-[2-[3-aminopropoxy]-ethoxy]-ethoxy]-propylcarbonate (31, 398 mg, 510 mol %). The reaction mixture was stirred at 80° C. for 16 hours. The volatiles were then removed in vacuo and the resulting residue was re-dissolved in DCM (15 mL). The organic solution was then extracted with water (10 mL), dried over sodium sulfate, filtered and concentrated to give 284 mg of the title compound 30 which was subsequently used without purification.

### Step B: Deprotection of paclitaxel-10-(deacetyl)10-{carbamoyl-3-[2-[2-[3-propoxy]-ethoxy]-ethoxy]-propylamino-benzylcarbamate} (30)

Compound 30 (284 mg, 0.2 mmol) was desylilated following the procedure in Ojima, I. et al. J. Med. Chem. (1997), 40:267. The residue so obtained (225 mg) was dissolved in methanol (20, mL) whereupon 10 wt % palladium on carbon (100 mg) was added. The resulting mixture was stirred for 40 minutes under one atmosphere of H₂. The reaction mixture was filtered through Celite and concentrated under reduced pressure. The residue so obtained was purified by preparative RP-HPLC (Method A). Fractions containing the appropriate mass, as determined by analytical HPLC-MS (Method B) were pooled and CH₃CN removed under reduced pressure. The remaining aqueous mixture was then lyophilized obtaining 140 mg of the desired paclitaxel-10-deacetyl,10-O-carbamoyl-PEG-amine of structure 32.

¹H NMR (CD₃OD, 300 MHz) δ 8.83 (d, J=8 Hz, 1H), 8.06 (d, J=8 Hz, 2H), 7.78 (d, J=8 Hz, 2H), 7.45 (m, 16H), 6.29 (s, 1H), 6.19 (t, 1H), 5.67 (m, 2H), 5.09 (s, 2H), 5.03 (d, J=10 Hz, 2H), 4.76 (d, J=6 Hz, 2H), 4.36 (m, 1H), 4.22 (s, 2H), 3.84 (d, J=7 Hz, 1H), 3.6 (m, 8H), 3.24 (m, 2H), 2.48 (m, 1H), 2.39 (s, 3H), 2.26 (m, 1H), 2.19 (s, 2H), 1.94 (m, 4H), 1.78 (m, 4H), 1.67 (s, 2H), 1.18 (s, 6H); Electrospray (LCMS) m/z 1192 (M+H⁺, C₆₄H₇₈N₃O₁₉ requires 1192); retention time 6.57 min. (1% to 99% B, Method B); (5) ¹H NMR (CD₃OD, 300 MHz) δ 8.38 (d, J=8 Hz, 1H), 8.14 (d, J=8 Hz, 2H), 7.89 (d, J=8 Hz, 2H), 7.45 (m, 11H), 6.29 (s, 1H), 6.19 (t, 1H), 5.66 (m, 2H), 5.03 (d, J=10 Hz, 2H), 4.76 (d, J=6 Hz, 2H), 4.35 (m, 1H), 4.22 (s, 2H), 3.85 (d, 1H), 3.60 (m, 8H), 3.12 (m, 2H), 2.50 (m, 1H), 2.40 (s, 3H), 2.26 (m, 1H), 2.19 (s, 2H), 1.94 (m, 4H), 1.82 (m, 4H), 1.68 (s, 2H), 1.18 (s, 6H); Electrospray (LCMS) m/z 1058 (M+H⁺, C₅₆H₇₂N₃O₁₇ requires 1058); retention time 5.07 min. (1% to 99% B, Method B).

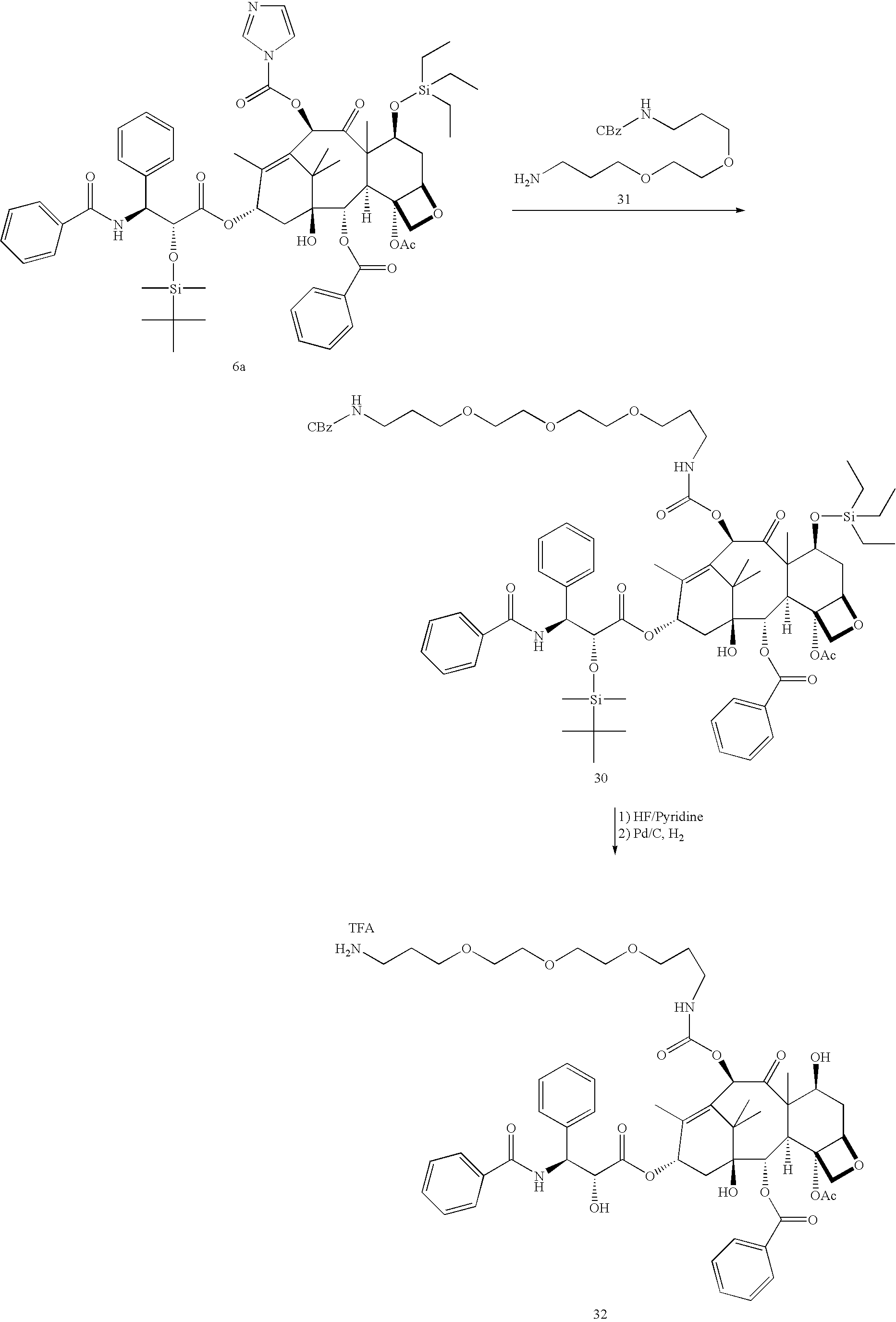

### iii). Reaction of paclitaxel-10-(deacetyl)10-O-(carbamoyl-PEG-amine) with HO—RFSGYIY—NHPv

To a paclitaxel-10-(deacetyl)10-O-(carbamoyl-PEG-amine) (32, 50 mg, 0.0426 mmol) prepared as above, dissolved in DMSO (1.0 mL) was added HO—RFSGYIY—NHPv (33, 47 mg, 100 mol %) followed by BOP (25 mg, 132 mol %) and DIEA (25 μL 336 mol %). The reaction mixture was stirred for 16 hours then directly injected onto a preparative RP-HPLC C-18 column for purification (Method A). Fractions containing the appropriate mass, as determined by analytical HPLC-MS (Method B) were pooled and CH₃CN removed under reduced pressure. The remaining aqueous mixture was then lyophilized to give 48.5 mg of paxlitaxel-linker-peptide conjugate of formula 34.

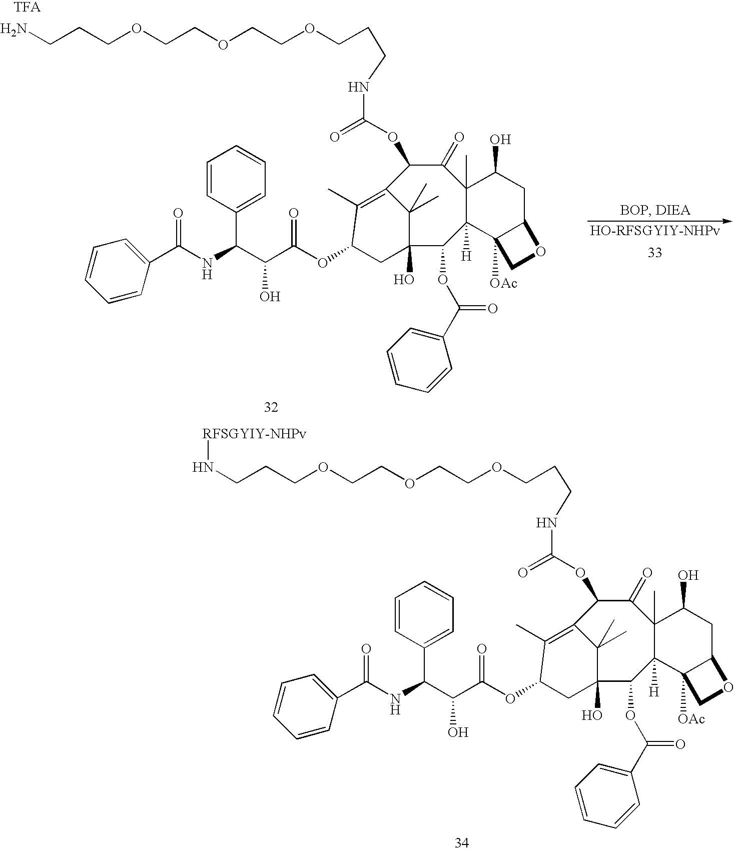

### Example 3

### Reaction of paclitaxel-10-(deacetyl)10-O-(carbamoyl-PEG-amine) with HO—K(Cbz)FSGYIYE(Bz1)-NHAc and deprotection

To a paclitaxel-10-(deacetyl)10-O-(carbamoyl-PEG-amine) 32, 77 mg, 0.066 mmol) prepared as above, dissolved in DMSO (3.0 mL) was added HO—K(Cbz)FSGYIYE(Bz1)-NH—Ac (35, 85 mg, 100 mol %) followed by BOP (46 mg, 150 mol %) and DIEA (48 μL, 420 mol %). The reaction mixture was stirred for 16 hours then directly injected onto a preparative RP-HPLC C-18 column for purification (Method A). Fractions containing the appropriate mass, as determined by analytical HPLC-MS (Method B) were pooled and CH₃CN removed under reduced pressure. The crude product was dissolved in MeOH (5 mL) and DMF (5 mL). To this were successively added a 1 N aqueous solution of HCl (100 μL) and 10 wt % palladium on carbon (79 mg). The reaction mixture was stirred at room temperature under 1 atm of H₂ for 16 hours. The reaction mixture was filtered through Celite and concentrated under reduced pressure. The product was dissolved in DMSO and injected onto a preparative RP-HPLC C-18 column for purification (Method A). Fractions containing the appropriate mass, as determined by analytical HPLC-MS (Method B) were pooled and CH₃CN removed under reduced pressure. The remaining aqueous mixture was then lyophilized to give 79 mg of paxlitaxel-linker-peptide conjugate of formula 36.

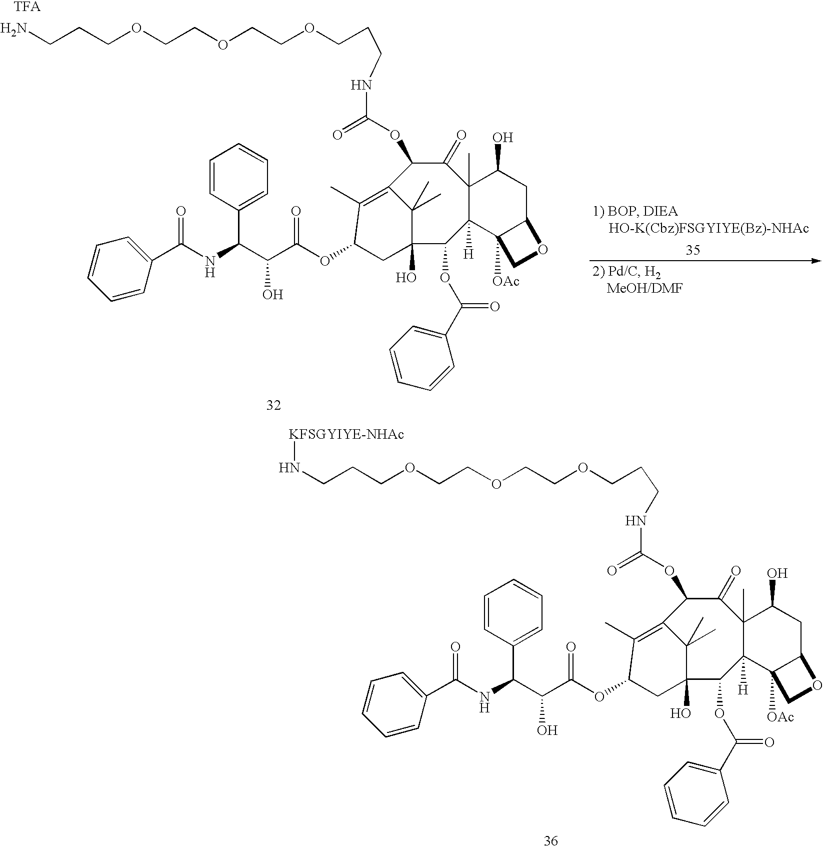

### Example 4

### i). Preparation of N-Boc-2-[2-[2-[2-aminoethoxy]ethoxy]ethoxy]ethylamine (38)

To the diaminoPEG 37 (0.5 g, 2.6 mmol), dissolved in CH₂Cl₂ (50 mL), were added the triethylamine (0.36 mL, 100 mol %) and the Boc₂O (0.55 g, 100 mol %). The reaction mixture was stirred for 4 hours and concentrated to dryness. The resulting residue was purified by silica gel column chromatography eluting with 9:1:0.1 chloroform:methanol:ammonium hydroxyde to give 0.26 g of the title compound 38.

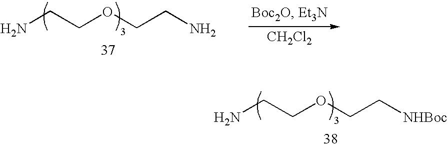

### ii). Reaction between 4-deacetyl-3-demethoxy-3-azidovinblastine and N-Boc-2-[2-[2-[2-aminoethoxy]ethoxy]ethoxy]ethylamine (41)

Step A: Preparation of 4-deacetyl-3-demethoxy-3-azidovinblastine (39) To a CH₂Cl₂ solution of 4-deacetyl-3-demethoxy-3-azidovinblastine, prepared according to the procedure in Ref: K. S. P. Bhushana Rao et al., J. Med. Chem. (1985), 28:1079, was added the N-Boc-2-[2-[2-[2-aminoethoxy]ethoxy]ethoxy]ethylamine 37 (0.2 g, 150 mol %), prepared as above, followed by DIEA (0.12 mL, 150 mol %). The reaction mixture was stirred at room temperature for 3 hours then concentrated in vacuo to give a residue that was purified by silica gel column chromatography eluting with 95:5 chloroform:methanol. The 4-deacetyl-3-demethoxy-3-(carboxamidyl-N—(N-Boc-2-[2-[2-[2-ethoxy]ethoxy]ethoxy]ethylamino])vinblastine intermediate was dissolved with a 1:1 mixture of DCM:TFA (60 mL each) and the mixture was stirred at room temperature for 10 minutes. The mixture was concentrated with a flow of N₂ and lyophilization gave 0.31 g of the title compound 39 which was used without further purification.

### Step B: Reaction of 4-deacetyl-3-demethoxy-3-(carboxamidyl-N—(N-Boc-2-[2-[2-[2-ethoxy]ethoxy]ethoxy]ethylamino])vinblastine intermediate with HO—K(B)FSGYIY—NHCbz and deprotection

To a 4-deacetyl-3-demethoxy-3-(carboxamidyl-N—(N-Boc-2-[2-[2-[2-ethoxy]ethoxy]ethoxy]ethylamino])vinblastine (39, 50 mg, 0.048 mmol) dissolved in DMSO (2.0 mL) was added HO—K(Boc)FSGYIY—NHCbz (40, 55 mg, 100 mol %) followed by BOP (30 mg, 140 mol %) and DIEA (36 μL, 440 mol %). The reaction mixture was stirred for 3 hours then directly injected onto a preparative RP-HPLC C-18 column for purification (Method A). Fractions containing the appropriate mass, as determined by analytical HPLC-MS (Method B) were pooled and CH₃CN removed under reduced pressure. The crude product was dissolved in CH₂Cl₂ (25 mL) and TFA (25 mL) and the mixture was stirred at room temperature for 10 minutes. The mixture was concentrated with a flow of N₂ and lyophilization gave 75 mg of the title compound 41.

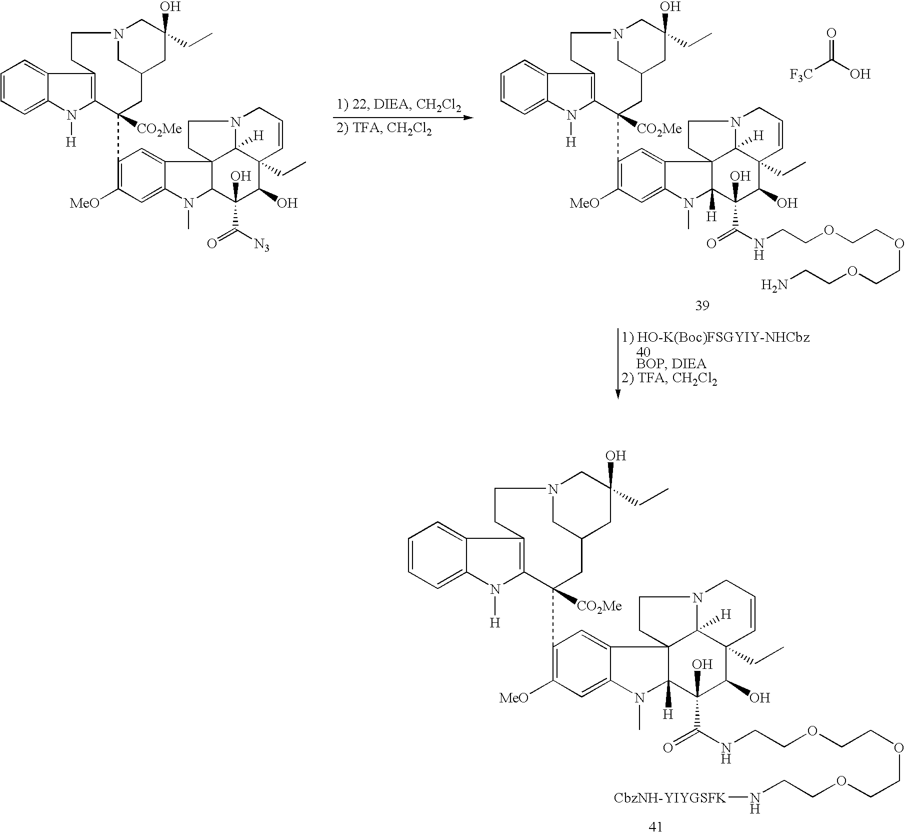

### Example 5

### Preparation of a vinblastine-linker-sphingosine conjugate with amide linker attachment at C3 of Vinblastine

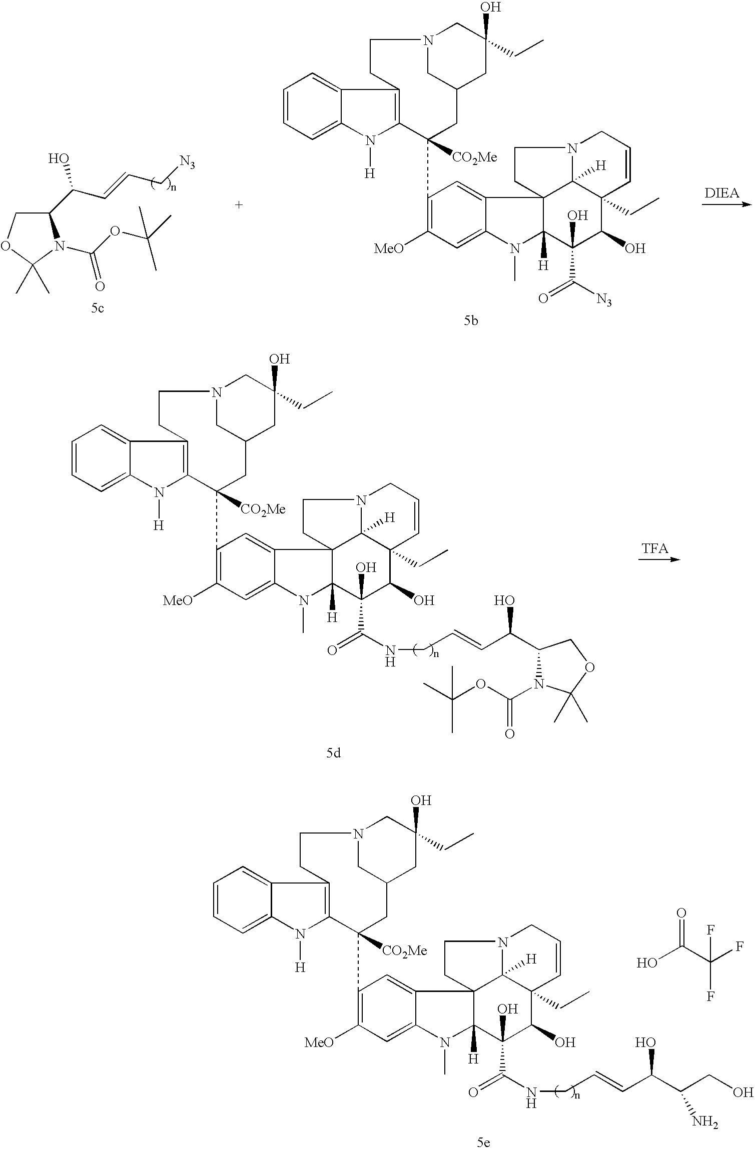

To a DCM solution of 4-deacetyl-3-demethoxy-3-azidovinblastine (5b), prepared as described elsewhere herein, is added neat, or in a solution of DCM, a head group protected ω-amino sphingosine TFA salt (5c, n=10, 150 mol %) prepared according to the procedure of Ettmayer, P. et al., Bioorg. Med. Chem. Lett. (2004), 14:1555 followed by DIEA (300 mol %). The reaction mixture is stirred for 3 h then concentrated in vacuo to give a residue that is purified by silica gel chromatography to give 5d (n=10). Compound 5d is dissolved in 10% aq. TFA solution and stirred for 1 h whereupon the solvents are evaporated. The residue is then dissolved in DMSO and injected onto a preparative RP-HPLC C-18 reversed phase column for purification (Method A) to give 5e (n=10) as a TFA salt.

### Example 6

### Preparation of a paclitaxel-linker-sphingosine conjugate with carbamate linker attachment at C10 of Paclitaxel

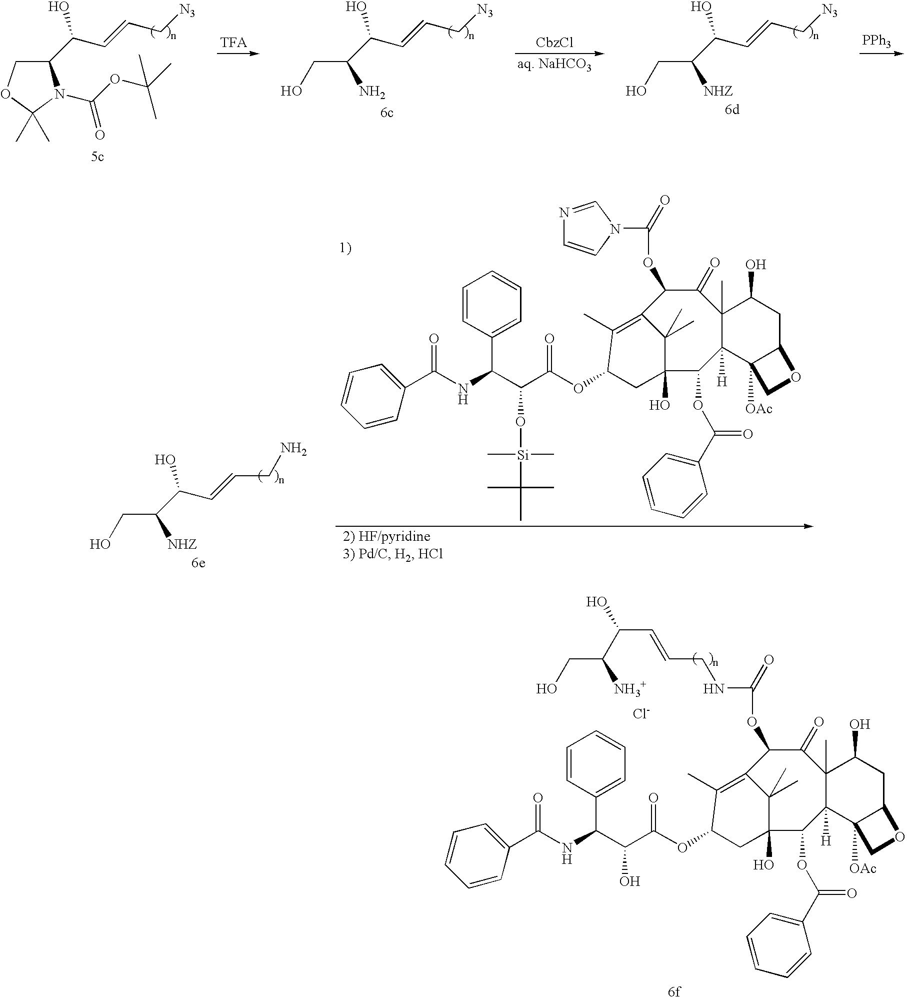

### i). Preparation of 2-benzyloxycarbonyl-ω-azido sphingosine (6d)

Head group protected ω-azido sphingosine 5c (n=10) prepared according to the procedure of Ettmayer, P. et al., Bioorg. Med. Chem. Lett. (2004), 14:1555 is dissolved in 10% aq. TFA solution and stirred for 1 h before the solvents are evaporated. The residue (crude 6c, n=10) is then dissolved in a mixture of 1:1 dioxane/10% aq. NaHCO₃. To the solution is added CBzCl (150 mol %) and the mixture is stirred for 2 h, then extracted with EtOAc. The organic layers are combined, dried over Na₂SO₄ and evaporated. The crude product is purified by silica gel chromatography eluting with a hexanes-ethyl acetate mixture to give 6d (n=10).

### ii). Preparation of 2-benzyloxycarbonyl-ω-amino sphingosine 6e

To 2-benzyloxycarbonyl-ω-azido sphingosine 6d (n=10) in 10% aq. THF is added PPh₃ and the mixture is stirred for 6 h at 60° C. The solvents are evaporated and the crude product is purified by silica gel chromatography eluting with a MeOH-EtOAc—NH₄OH mixture to give 6e (n=10).

### iii). Preparation of a paclitaxel-linker-sphingosine conjugate with linker attachment at C10 of paclitaxel

To 2′-O-(tert-butyldimethylsilyl)-7-O-(triethylsilyl)-10-O-deacetyl-10-O-(carbonylimidazolyl)paclitaxel Paclitaxel-2′-(tert-butlyldimethylsilyl)-7-(triethylsilyl)-10-(deacetyl-carbonylimidazole) (100 mol %), prepared as above, dissolved in anhydrous isopropyl alcohol is added 2-benzyloxycarbonyl-co-amino sphingosine 6e (n=10, 300 mol %). The reaction mixture is stirred under reflux for 16 hours. The volatiles are then removed in vacuo and the resulting residue is re-dissolved in DCM. The organic solution is then extracted with water and dried over Na₂SO₄. After filtration and evaporation of the volatiles the residue is desalinated following the procedure in Ojima, I. et al. J. Med. Chem. (1997), 40:267. The residue so obtained is dissolved in a 7:3 mixture of THF/water, whereupon 10 wt % palladium on carbon and HCl (100 mol %, introduced as a 1 M aqueous solution), is added. The resulting mixture is shaken under 60 psi of H₂. The reaction mixture is filtered through Celite and concentrated under reduced pressure and lyophilized. The residue so obtained is purified by preparative RP-HPLC (Method A). Fractions containing the appropriate mass, as determined by analytical HPLC-MS (Method B) are pooled and CH₃CN removed under reduced pressure. The remaining aqueous mixture is then lyophilized to give 6f (n=10).

Several conjugates have been prepared by following the procedures described herein and slight modifications thereof. Table 6 provide mass spectroscopy data for exemplary conjugates.

TABLE 6

| | | | | | | Retention |
|---|---|---|---|---|---|---|
| | | | | | | Time (min) |
| | | | | MS | | (HPLC |
| Systematic Name | Formula | Mol Weight | Purity | Expected | MS Observed | Method B) |
| CBz-RPRTSSF-PEG(13)- | C104H136F6N16O33 | 2252.2962 | 99% | 2024(M + H) | 2024(M + H) | 5.24 |
| 10Ca-PXL x 2TFA | | | | | | |
| Pv-GRPRTSSFAE(Bzl)G- | C120H164F6N20O37 | 2592.7178 | 95% | 2378(M + H) | 2378(M + H) | 5.37 |
| PEG(13)-10Ca-PXL x 2TFA | | | | | | |
| Pv-GRPRTSSFAEG-PEG(13)- | C111H155F3N20O36 | 2402.5531 | 96% | 2288(M + H) | 2288(M + H) | 5.03 |
| 10Ca-PXL xTFA | | | | | | |
| Pv-GRPRTSsFAEG-PEG(13)- | C113H157DF6N20O38 | 2519.5989 | 95% | 2288(M + H) | 2288(M + H) | 4.97 |
| 10Ca-PXL x 2TFA | | | | | | |
| Pv-GRPRAAAFAEG-PEG(13)- | C112H154F6N20O35 | 2454.552 | 95% | 2226(M + H) | 2226(M + H) | 5.04 |
| 10Ca-PXL x 2TFA | | | | | | |
| Ac-RPRTSSF-PEG(13)-10Ca- | C98H132F6N16O32 | 2160.1992 | 98% | 1932(M + H) | 1932(M + H) | 4.99 |
| PXL x 2TFA | | | | | | |
| BOC-RPRTSSF-PEG(13)- | C101H138F6N16O33 | 2218.279 | 98% | 1990(M + H) | 1990(M + H) | 5.16 |
| 10Ca-PXL x 2TFA | | | | | | |
| Ac-RSRTSSF-PEG(13)-10Ca- | C99H136F6N16O33 | 2192.2412 | 99% | 1964(M + H) | 1964(M + H) | 5.07 |
| PXL x 2TFA | | | | | | |
| Pv-RSRKESY-PEG(13)-10Ca- | C103H143F6N17O34 | 2277.3466 | 99% | 2049(M + H) | 2049(M + H) | 4.74 |
| PXL x 2TFA | | | | | | |
| Pv-RSRTSSFAEG-PEG(13)- | C109H151F6N19O38 | 2449.4868 | 93% | 2221(M + H) | 2221(M + H) | 4.94 |
| 10Ca-PXL x 2TFA | | | | | | |
| Pv-GRSRTSSFAEG-PEG(13)- | C111H154F6N20O39 | 2506.5386 | 99% | 2278(M + H) | 2278(M + H) | 4.96 |
| 10Ca-PXL x 2TFA | | | | | | |
| H-GIYWHHY-ALK(5)-7Es-PXL | C102H118N14O24 | 1924.1336 | >90% | 1923(M + H) | 1923(M + H) | |
| CBz-GIYWHHY-ALK(6)-7Ca- | C111H127N15O26 | 2087.3092 | >95% | | | 12.83 |
| PXL | | | | | | |
| H-GIYWHHY-ALK(6)-7Ca-PXL | C103H122CIN15O24 | 1989.6359 | >90% | 1953(M + H) | 1953(M + H) | |
| x HCl | | | | | | |
| CBz-YIYGSFK(CBz)-ALK(6)- | C111H130N10O28 | 2052.2982 | >95% | 2052(M + H) | 2052(M + H) | |
| 10Es-PXL | | | | | | |
| H-YIYGSFK-ALK(6)-10Es-PXL | C95H120Cl2N10O24 | 1856.9516 | >95% | 1784(M + H) | 1784(M + H) | |
| x 2 HCl | | | | | | |
| Ac-YIYGSFK-PEG(13)-10Ca- | C104H132F3N11O30 | 2073.2377 | 97% | 1959(M + H) | 1959(M + H) | 5.51 |
| PXL x TFA | | | | | | |
| Ac-E(Bzl)YIYGSFK(CBz)- | C122H150N12O33 | 2312.5876 | 90% | 2312(M + H) | 2334(M + Na) | 6.95 |
| PEG(13)-10Ca-PXL | | | | | | |
| Pv-YIYGSFR-PEG(13)-10Ca- | C108H140F3N13O29 | 2141.3589 | >95% | 2030(M + H) | 2030(M + H) | 5.82 |
| PXL x TFA | | | | | | |
| Pv-YIYGSFR-PEG(13)-10Ca- | C108H141DF3N13O29 | 2144.3808 | 95% | 2030(M + H) | 2030(M + H) | 5.82 |
| PXL x TFA | | | | | | |
| Pv-YIFGSFR-PEG(13)-10Ca- | C108H140F3N13O28 | 2125.3595 | 98% | 2011(M + H) | 2011(M + H) | 6.2 |
| PXL x TFA | | | | | | |
| Ac-EYIYGSFK-PEG(13)-10Ca- | C109H139F3N12O33 | 2202.3529 | >95% | 2088(M + H) | 2110(M + Na) | 5.43 |
| PXL x TFA | | | | | | |
| Ac-EYIFGSFK-PEG(13)-10Ca- | C109H139F3N12O32 | 2186.3535 | 94% | 2072(M + H) | 2072(M + H) | 5.7 |
| PXL x TFA | | | | | | |
| Ac-EYIyGSFK-PEG(13)-10Ca- | C109H140DF3N12O33 | 2205.3748 | 95% | 2088(M + H) | 2088(M + H) | 5.45 |
| PXL x TFA | | | | | | |
| Ac-EYIYGSFK(CBz)-PEG(13)- | C115H144N12O33 | 2222.4632 | 95% | 2222(M + H) | 2244(M + Na) | 6.33 |
| 10Ca-PXL | | | | | | |
| Pv-E(Bzl)YIYGSFK(CBz)- | C119H151F3N14O33 | 2362.5711 | 95% | 2250(M + H) | 2250(M + H) | 6.31 |
| PEG(13)-10Ca-PXL x TFA | | | | | | |
| Pv-EYIYGSFR-PEG(13)-10Ca- | C110H144N14O31 | 2158.4228 | 98% | 2158(M + H) | 2158(M + H) | 5.75 |
| PXL | | | | | | |
| Ac-YIYGSFR-PEG(13)-10Ca- | C104H132F3N13O30 | 2101.2511 | 98% | 1987(M + H) | 1987(M + H) | 5.57 |
| PXL x TFA | | | | | | |
| Ac-EYIYGSFR-PEG(13)-10Ca- | C107H138N14O31 | 2116.3424 | 88% | 2116(M + H) | 2116(M + H) | 5.46 |
| PXL | | | | | | |
| Pv-YIYGSFR-PEG(13)-10Ca- | C108H140F3N13O29 | 2141.3589 | 91% | 2029(M + H) | 2029(M + H) | 5.79 |
| PXL x TFA | | | | | | |
| Pv-RLVAYE(Bzl)GYV- | C122H161F3N16O32 | 2420.6971 | 99% | | | 6.43 |
| PEG(13)-10Ca-PXL x TFA | | | | | | |
| Pv-RLVAYEGYV-PEG(13)- | C113H154N16O30 | 2216.5488 | 95% | 2216(M + H) | 2216(M + H) | 5.98 |
| 10Ca-PXL | | | | | | |
| PXL-10Ca-ALK(10)- | C63H82F3N3O18 | 1226.3453 | 99% | 1112(M + H) | 1112(M + H) | 5.89 |
| sphinganine x TFA | | | | | | |
| PXL-7Ca-ALK(10)-sphinganine | C65H84F3N3O19 | 1268.3825 | 99% | 1154(M + H) | 1154(M + H) | 6.31 |
| x TFA | | | | | | |
| Pv-ARDIKYD-PEG(13)-10Ca- | C103H140F6N14O34 | 2232.3028 | 94% | 2004(M + H) | 2004(M + H) | 5.08 |
| PXL x 2TFA | | | | | | |
| CBz-RPRTSSF-PEG(11)-3Am- | C99H137F6N19O26 | 2123.2734 | 99% | 1895(M + H) | 1895(M + H) | 3.99 |
| VBL x 2TFA | | | | | | |
| CBz-GRPRTSSFAE(Bzl)G- | C114H159N23O28 | 2299.6474 | >95% | 2299(M + H) | 2299(M + H) | 4.65 |
| 3Am-VBL | | | | | | |
| Pv-GRPRTSSFAE(DMAB)G- | C129H186F6N24O33 | 2715.0198 | 94% | 2470(M + H) | 2470(M + H) | 4.66 |
| 3Am-VBL x 2TFA | | | | | | |
| Pv-GRPRTSSFAEG-3Am-VBL | C108H157F6N23O31 | 2387.5542 | 91% | 2159(M + H) | 2159(M + H) | 3.76 |
| x 2TFA | | | | | | |
| CBz-RPRTSSF-PEG(29)-3Am- | C111H161F6N19O32 | 2387.5914 | 95% | 2159(M + H) | 2159(M + H) | 4.2 |
| VBL x 2TFA | | | | | | |
| Ac-RPRTSSF-PEG(11)-3Am- | C93H133F6N19O25 | 2031.1764 | 95% | 1802(M + H) | 1802(M + H) | 3.67 |
| VBL x 2TFA | | | | | | |
| BOC-RPRTSSF-PEG(11)-3Am- | C96H139F6N19O25 | 2073.2568 | 95% | 1861(M + H) | 1861(M + H) | 3.9 |
| VBL x 2TFA | | | | | | |
| Ph(C=O)-RPRTSSF-PEG(11)- | C98H135F6N19O25 | 2093.2472 | 93% | 1865(M + H) | 1865(M + H) | 3.8 |
| 3Am-VBL x 2TFA | | | | | | |
| Ac-GRPRTSSFAEG-3Am-VBL | C103H150F3N23O29 | 2231.4499 | 98% | 2117(M + H) | 2117(M + H) | 3.62 |
| x TFA | | | | | | |
| CBz-GRPRTSSFAEG-3Am- | C111H155F6N23O32 | 2437.5708 | 91% | 2209(M + H) | 2209(M + H) | 3.92 |
| VBL x 2TFA | | | | | | |
| Pv-RSRTSSF-PEG(11)-3Am- | C94H137F6N19O26 | 2063.2184 | 92% | 1835(M + H) | 1835(M + H) | 3.73 |
| VBL x 2TFA | | | | | | |
| VBL-3Am-ALK(10)-Sphingosine | C60H85F3N6O11 | 1123.3603 | 93% | 1010(M + H) | 1010(M + H) | 4.31 |
| x TFA | | | | | | |
| H-YIYGSFK-PEG(11)-3Am- | C99H132F6N14O24 | 2016.2016 | >95% | 1788(M + H) | 1788(M + H) | 6.35* |
| VBL x 2TFA | | | | | | |
| BOC-YIYGSFK(BOC)- | C105H146N14O24 | 1988.3878 | 91% | 1988(M + H) | 1988(M + H) | 5.36 |
| PEG(11)-3Am-VBL | | | | | | |
| BOC-YIYGSFK(BOC)- | C117H170N14O30 | 2252.7058 | >95% | 2251(75%) | 2253(M + H + 1) | 7.60* |
| PEG(29)-3Am-VBL | | | | | | |
| H-YIYGSFK-PEG(29)-3Am- | C111H156F6N14O30 | 2280.5196 | >95% | 2052(M + H) | 2052(M + H) | 6.30* |
| VBL x 2TFA | | | | | | |
| BOC- | C105H147N14O27P | 2068.3677 | | | | |
| YI(phospho)YGSFK(BOC)- | | | | | | |
| PEG(11)-3Am-VBL | | | | | | |
| H-YI(phospho)YGSFK- | C99H133F6N14O27P | 2096.1815 | | | | |
| PEG(11)-3Am-VBL x 2TFA | | | | | | |
| CBz-YIYGSFK(BOC)-PEG(11)- | C108H144N14O24 | 2022.405 | 96% | 2022(M + H) | 2022(M + H) | 5.7 |
| 3Am-VBL | | | | | | |
| CBz-YIYGSFK-PEG(11)-3Am- | C105H137F3N14O24 | 2036.3119 | 91% | 1922(M + H) | 1922(M + H) | 4.99 |
| VBL x TFA | | | | | | |
| CBz-YIFGSFK-PEG(11)-3Am- | C105H137F3N14O23 | 2020.3125 | 94% | 1906(M + H) | 1906(M + H) | 5.04 |
| VBL x TFA | | | | | | |
| CBz-YIYGSFK-PEG(11)-3Am- | C105H138DF3N14O24 | 2039.3338 | 95% | 1922(M + H) | 1922(M + H) | 4.75 |
| VBL x TFA | | | | | | |
| Ac-YIFGSFK(BOC)-PEG(11)- | C102H140N14O23 | 1930.308 | 95% | 1930(M + H) | 1930(M + H) | 4.9 |
| 3Am-VBL | | | | | | |
| Ac-YIFGSFK-PEG(11)-3Am- | C99H133F3N14O23 | 1944.2149 | 95% | 1830(M + H) | 1830(M + H) | 4.15 |
| VBL x TFA | | | | | | |
| CBz-E(Bzl)YIFGSFK(BOC)- | C117H159N15O27 | 2207.6274 | 95% | 2207(M + H) | 2207(M + H) | 5.89 |
| PEG(11)-3Am-VBL | | | | | | |
| CBz-E(Bzl)YIFGSFK-PEG(11)- | C110H144F3N15O27 | 2165.4271 | 92% | 2051(M + H) | 2051(M + H) | 4.68 |
| 3Am-VBL x TFA | | | | | | |
| Ac-YIYGSFR-PEG(11)-3Am- | C99H133F3N16O23 | 1972.2283 | 99% | 1858(M + H) | 1858(M + H) | 4.12 |
| VBL x TFA | | | | | | |
| Pv-YIYGSFR-PEG(11)-3Am- | C102H139F3N16O23 | 2014.3087 | 95% | 1900(M + H) | 1900(M + H) | 4.52 |
| VBL x TFA | | | | | | |
| BOC-EYIYGSFK(BOC)- | C110H153N15O27 | 2117.503 | 94% | 2117(M + H) | 2117(M + H) | 5.18 |
| PEG(11)-3Am-VBL | | | | | | |
| Pv-E(DMAB)YIYGSFR- | C127H171F3N18O28 | 2454.8469 | 99% | 2340(M + H) | 2340(M + H) | 5.49 |
| PEG(11)-3Am-VBL x TFA | | | | | | |
| Pv-EYIYGSFR-PEG(11)-3Am- | C105H145N17O24 | 2029.4 | 99% | 2029(M + H) | 2029(M + H) | 4.5 |
| VBL | | | | | | |
| BOC-YIYGSFR-PEG(11)-3Am- | C102H139F3N16O24 | 2030.3081 | 97% | 1916(M + H) | 1916(M + H) | 4.57 |
| VBL x TFA | | | | | | |
| BOC-E(Bzl)YIFGSFK(BOC)- | C117H159N15O27 | 2207.6274 | 94% | 2207(M + H) | 2207(M + H) | 5.9 |
| PEG(11)-3Am-VBL | | | | | | |
| CBz-YIYGSFK(CBz)-PEG(11)- | C111H142N14O24 | 2056.4222 | 97% | 2056(M + H) | 2056(M + H) | 5.52 |
| 3Am-VBL | | | | | | |
| BOC-YIYGSFS-PEG(11)-3Am- | C97H131N13O23 | 1847.1752 | 99% | 1847(M + H) | 1847(M + H) | 4.94 |
| VBL | | | | | | |
| Ac-EYIYGSFR-PEG(11)-3Am- | C104H140F3N17O26 | 2101.3435 | 99% | 1987(M + H) | 1987(M + H) | 4.22 |
| VBL x TFA | | | | | | |
| CH3O(CH2CH2O)3CH2CH2(C═ | C102H141N13O26 | 1965.3074 | 97% | 1965(M + H) | 1965(M + H) | 4.51 |
| O)YIYGSFS-PEG(11)-3Am- | | | | | | |
| VBL | | | | | | |
| Ac-YIYGSFS-PEG(11)-3Am- | C94H125N13O22 | 1789.0954 | 94% | 1789(M + H) | 1789(M + H) | 4.39 |
| VBL | | | | | | |
| Ac-YIYGSFH-PEG(11)-3Am- | C99H128F3N15O23 | 1953.1821 | 99% | 1839(M + H) | 1839(M + H) | 4.18 |
| VBL x TFA | | | | | | |
| CH3O(CH2CH2O)3CH2CH2(C═ | C107H144F3N15O27 | 2129.3941 | 98% | 2015(M + H) | 2015(M + H) | 4.29* |
| O)YIYGSFH-PEG(11)-3Am- | | | | | | |
| VBL x TFA | | | | | | |
| CBz-GIYWHHY-PEG(11)-3Am- | C108H135N19O20 | 2019.3698 | >95% | 2018(83.9%) | 2018 | 6.91* |
| VBL | | | | | | |
| BOC-GIYWHHY-PEG(11)- | C105H136N18O21 | 1986.3374 | >95% | 1986(M + H) | 1986(M + H) | 6.67* |
| 3Am-VBL | | | | | | |
| H-GIYWHHY-PEG(11)-3Am- | C102H129F3N18O21 | 2000.2443 | >95% | 1886(M + H) | 1886(M + H) | 6.13* |
| VBL x TFA | | | | | | |
| BOC-GIYWHHY-PEG(29)- | C117H160N18O27 | 2250.6554 | >95% | 2250(M + H) | 2250(M + H) | 6.68* |
| 3Am-VBL | | | | | | |

### Example 7

### Src and Akt Kinase Assays

Human Src (#14-326) and Akt (#14-276) kinases were purchased from Upstate (Charlottesville, Va.). Kinase reactions were carried out in 50 μl kinase reaction cocktail (25 mM Tris-HCl, pH 7.5, 5 μM β-glycerophosphate, 2 mM DTT, 0.1 mM Na3VO4, 10 mM MgCl2, 1 mg/ml BSA, 40 mM ATP, 0.5 to 1.0 units enzyme). Substrates (peptide or drug-peptide conjugate in 1 ml) were added (25, 50 and 100 μM) and the reaction was incubated for 2-5 hours at 30° C. PKLight reagent (23 μl) (Cambrex BioSciences, Rockland, Me.) was mixed with 46 ml of the above kinase reaction and ATP utilization relative to no substrate and no ATP controls was determined by measuring luminescence with a SpectraMax Gemini EM plate reader (Molecular Devices, Sunnyvale, Calif.). Peptide substrates for Src and Akt have been described. For examples, see Lou et al. Letters in Peptide Science, 2, 289-296 (1995); Lou et al. Bioorganic & Medicinal Chemistry, 4, 677-682 (1996); Alessi et al. FEBS Letters, 399, 333-338 (1996). Substrate phosphorylation potential was determined from the linear portion of the substrate concentration dose response as a percentage of the activity observed with the parent peptide used for drug conjugation.

### Example 8

### Fluorescence-based assays for enhancement (paclitaxel) and inhibition (vinblastine) of tubulin polymerization

The assay kit (#BK011) was purchased from Cytoskeleton (Denver, Colo.). The assays were carried out according to the manufacturer's instructions, except that 1 mg/ml BSA (Sigma #A3059) was included in all assays. Paclitaxel assays were carried out in the absence of glycerol and vinblastine assays were carried out in the presence of 20% glycerol. Parent drugs and conjugates were tested at 0.75, 1.5, 3 and 10 micromolar final concentration and results represent a comparison of conjugate and parent drug curves obtained from the linear range of the dose responses. Mean percentages of paclitaxel or vinblastine activity

(±SD) represent the average of all tests carried out for all lots of a given compound.

### Example 9

### Topoisomerase II Assay

Doxorubicin derivatives were assayed for their affect on Topoisomerase II using the Topoisomerase II Drug Screening Kit (Catalog # 1009-1) produced by TopoGEN Inc. (Columbus, Ohio). Specifically the kit was used to assay whether Doxorubicin derivatives altered the ability of Topoisomerase II to catalyze the formation of relaxed conformation DNA from a super-coiled plasmid. Doxorubicin derivatives were compared directly to Doxorubicin at 10, 3, 1, 0.3, 0.1 and 0.03 micromolar concentrations. The quantity of relaxed conformation DNA was quantified from an agarose gel on which is it is separated from the super-coiled DNA by standard electrophoresis. The more active a drug is at a particular concentration the less relaxed conformation DNA is produced by the action of Topoisomerase II. The results are presented in terms of percent activity of Doxorubicin.

### Example 10

Cytotoxicity Assay (Monolayer)

Monolayer assays with tumor cell lines (MCF-7 breast carcinoma and HT-29 colorectal carcinoma from ATCC) were carried out in triplicate in 96-well plates with RPMI1640 medium containing 5% fetal bovine serum, 100 U/ml penicillin and 100 μg/ml streptomycin. Normal human foreskin fibroblasts (HFF #CC-2509) were from Cambrex and were cultured in FGM-2 medium. Exponentially growing cells (5,000 MCF-7 or HT-29; 1,500 HFF) were plated in 100 μl medium and incubated overnight (5% CO2, 37° C.). Compounds (20 μM to 20 μM final concentration, 6-8 doses) and vehicle (DMSO) controls were added and the incubation was continued for an additional 72 hours. Final cell density was determined by incubating cultures with 25 μl AlamarBlue reagent (BioSource, Camarillo, Calif.) for 4 hours, followed by determination of fluorescence at excitation of 544 nm and emission of 590 nm with a SpectroMax Gemini EM fluorescence plate reader (Molecular Devices, Sunnyvale, Calif.). EC50 values were generated from dose-response curves by a 4-parameter method using Softmax PRO software. Mean EC50s (±SD) represent the average of all tests carried out for all lots of a given compound. Outlier EC50 values (<7%) were identified and removed prior to analysis using the method of Hoaglin et al., J. Amer. Statistical Assoc., 81, 991-999 (1986).

Cytotoxicity Assay (Soft Agar)

Assays were carried out in 24-well plates with 0.5 ml bottom layers (0.8% agar) and 0.5 ml top layers (0.38% agar) in RPMI1640 medium containing 5% fetal calf serum. Top layers were plated with 1,250 MCF-7 or 5,000 HT-29 cells per well and drugs, compounds or vehicle controls in triplicate as described above. Plates were incubated as above for 10-14 days and then colony formation was assessed by adding 50 μl AlamarBlue to each well and determining EC50s as described above for monolayer assays.

### Example 11

Serum Stability

The stability of conjugates was measured in RPMI1640 cell culture medium containing 10% fetal bovine serum. The serum-containing medium was pre-warmed at 37° C. for 3 min prior to addition of test articles. Test articles, prepared in DMSO as 5 mM stocks, were added to the cell culture media to a final concentration of 10 μM. Aliquots (150 ml) were withdrawn in triplicate at 0, 4, 8, 24 and 72 hours and combined with the same volume of ice-cold acetonitrile to terminate the reaction. The mixture was centrifuged at 2,000×g for 10 minutes. One part of the supernatant was mixed with four parts of deionized water to bring down the percentage of organic solvent. The diluted samples were then assayed by LC/MS for the test article. The natural log of the percent remaining was plotted versus time. A linear fit was used to determine the rate constant. The fit was truncated after the percent of remaining test article was less than 10%. The elimination half-lives associated with the disappearance of test articles were determined to compare their relative stability. The assays were carried out by Absorption Systems (Exton, Pa.).

### Example 12

Liver Microsome Metabolic Stability

Human and mouse liver microsomes were obtained from Absorption Systems (Exton, Pa.) and Xenotech (Lenexa, Kans.), respectively. The reaction mixture contained microsomes (human or mouse) 1.0 mg/ml, potassium phosphate, pH 7.4 100 mM, magnesium chloride 10 μM, test article 10 mM, and was equilibrated at 37° C. for 3 min. The reaction was initiated by adding NADPH (1 mM final), and the system was then incubated in a shaking water bath at 37° C. Aliquots (100 μl) were withdrawn in triplicate at 0, 15, 30, and 60 minutes and combined with 900 μl of ice-cold 50/50 acetonitrile/dH2O to terminate the reaction. Two controls (testosterone and propranolol) were run simultaneously with the test articles in separate reactions. The samples were assayed by LC/MS for the test article. The natural log of the percent remaining was plotted versus time. A linear fit was used to determine the rate constant. The fit was truncated when percent remaining of the test article was less than 10%. The elimination half-lives associated with the disappearance of test and control articles were determined to compare their relative metabolic stability. The assays were carried out at Absorption Systems (Exton, Pa.).

What is claimed is:

1. A conjugate, comprising a drug and a substrate for a protein kinase or a lipid kinase non-releasably linked thereto, optionally via a non-releasable linker, or a pharmaceutically acceptable derivative thereof.

2. The conjugate of claim 1, wherein a significant fraction of a biological activity of the drug is retained in the conjugate.

3. The conjugate of claim 1, wherein more than 50% of the biological activity is retained in the conjugate.

4. The conjugate of claim 1, wherein more than 20% of the biological activity is retained in the conjugate.

5. The conjugate of claim 1, wherein more than 5% of the biological activity is retained in the conjugate.

6. The conjugate of claim 1 that comprises:
(substrate)t, (Linker)q, and (drug)d;
wherein at least one substrate moiety is linked, optionally via a non-releasable linker to at least one drug, t is 1 to 6, q is 0 to t, and d is 1 to 6.

7. The conjugate of claim 1, wherein the kinase is overexpressed, overactive or exhibits undesired activity in a target system.

8. The conjugate of claim 1, wherein the kinase is associated with an ACAMPS-related condition.

9. The conjugate of claim 1, wherein the substrate is a substrate for a protein kinase.

10. The conjugate of claim 9, wherein the protein kinase is selected from AFK, Akt, AMP—PK, Aurora kinase, beta-ARK, Abl, ATM, ATR, CAK, Cam-II, Cam-III, CCD, Cdc2, Cdc28-dep, CDK, Flt, Fms, Hck, CKI, CKII, Met, DnaK, DNA-PK, Ds-DNA, EGF—R, ERA, ERK, ERT, FAK, FES, FGR, FGF—R, Fyn, Gag-fps, GRK, GRK2, GRK5, GSK, H4-PK-1, IGF—R, IKK, INS—R, JAK, KDR, Kit, Lck, MAPK, MAPKKK, MAPKAP2, MEK, MEK, MFPK, MHCK, MLCK, p135tyk2, p37, p38, p70S6, p74Raf-1, PDGF—R, PD, PhK, PI3K, PKA, PKC, PKG, Raf, PhK, RS, SAPK, Src, Tie-2, m-TOR, TrkA, VEGF—R, YES and ZAP-70.

11. The conjugate of claim 10, wherein the protein kinase is Akt, Abl, CAK, Cdc2, Fms, Met, EGF—R, ERK1, ERK2, FAK, Fyn, IGF—R, Lck, p70S6, PDGF—R, PI3K, PKA, PKC, Raf, Src, Tie-2 or VEGF—R.

12. The conjugate of claim 10, wherein the protein kinase is Akt or Src.

13. The conjugate of claim 1, wherein the substrate is a peptide substrate containing natural and/or non-natural amino acids.

14. The conjugate of claim 1, wherein the kinase is a lipid kinase.

15. The conjugate of claim 14, wherein the lipid kinase is selected from phosphoinositol kinase, diacylglycerol kinase and sphingosine kinase.

16. The conjugate of claim 14, wherein the lipid kinase is sphingosine kinase.

17. The conjugate of claim 1, wherein the substrate is phosphorylated by a kinase selected from Akt, Abl, CAK, Cdc2, Fms, Met, EGF—R, ERK1, ERK2, FAK, Fyn, IGF—R, Lck, p70S6, PDGF—R, PI3K, PKA, PKC, Raf, Src, Tie-2, VEGF—R and sphingosine kinase.

18. The conjugate of claim 1, wherein the substrate is phosphorylated by a kinase selected from Akt and Src.

19. The conjugate of claim 1, wherein the drug is a cytotoxic agent.

20. The conjugate of claim 1, wherein the drug is a lable.

21. The conjugate of claim 1, wherein the drug is not a rare earth cryptate containing moiety.

22. The conjugate of claim 1, wherein the drug is not a peptide.

23. The conjugate of claim 1, wherein the drug is an anti-infective agent, antihelminthic agent, antiprotozoal agent, antimalarial agent, antiamebic agent, antileiscmanial agent, antitrichomonal agent, antitrypanosomal agent, sulfonamide, antimycobacterial agent, or antiviral agent.

24. The conjugate of claim 1, wherein the drug is an alkylating agent, plant alkaloid, antimetabolite, antibiotic, microtubule or tubulin binding agent.

25. The conjugate of claim 1, wherein the drug is selected from a central nervous system depressant or stimulant, respiratory tract drug, pharmacodynamic agent, cardiovascular agent, blood or hemopoietic system agent, gastrointestinal tract agent, and locally acting chemotherapeutic agent.

26. The conjugate of claim 1, wherein the drug is selected from among the following classes of drugs:
a) anthracycline family of drugs,
b) vinca alkaloid drugs,
c) mitomycins,
d) bleomycins,
e) cytotoxic nucleosides,
f) pteridine family of drugs,
g) diynenes,
h) estramustine,
i) cyclophosphamide,
j) taxanes,
k) podophyllotoxins,
l) maytansanoids,
m) epothilones, and
n) combretastatin and analogs,
or pharmaceutically acceptable derivatives thereof.

27. The conjugate of claim 1, wherein the drug is selected from among the following drugs:
a) doxorubicin,
b) carminomycin,
c) daunorubicin,
d) aminopterin,
e) methotrexate,
f) methopterin,
g) dichloromethotrexate,
h) mitomycin C,
i) porfiromycin,
j) 5-fluorouracil,
k) 6-mercaptopurine,
l) cytosine arabinoside,
m) podophyllotoxin,
n) etoposide,
o) etoposide phosphate,
p) melphalan,
q) vinblastine,
r) vincristine,
s) leurosidine,
t) vindesine,
u) estramustine,
v) cisplatin,
w) cyclophosphamide,
x) paclitaxel,
y) leurositte,
z) 4-desacetylvinblastine,
aa) epothilone B,
bb) docetaxel,
cc) maytansanol,
dd) epothilone A, and
ee) combretastatin and analogs;
or a pharmaceutically acceptable derivative thereof.

28. The conjugate of claim 1 comprising a non-releasable linker.

29. The conjugate of claim 1, wherein the linker comprises linear or acyclic portions, cyclic portions, aromatic rings or combinations thereof.

30. The conjugate of claim 1, wherein the linker comprises linear or acyclic portions.

31. The conjugate of claim 1, wherein the linker comprises up to 50 main chain atoms.

32. The conjugate of claim 1, wherein the linker comprises up to 40 main chain atoms.

33. The conjugate of claim 1, wherein the linker comprises up to 30 main chain atoms.

34. The conjugate of claim 1, wherein the linker comprises up to 20 main chain atoms.

35. The conjugate of claim 1, wherein the linker comprises up to 10 main chain atoms.

36. The conjugate of claim 1, wherein the linker comprises up to 5 main chain atoms.

37. The conjugate of claim 1, wherein the linker comprises oligomers of ethylene glycol or straight alkelene chains or mixtures thereof.

38. The conjugate of claim 1, wherein the linker comprises polyethylene glycol.

39. The conjugate of claim 38, wherein the polyethylene glycol comprises 5, 11, 13, 14, 22 or 29 atoms in the chain.

40. The conjugate of claim 39, wherein the polyethylene glycol comprises 5, 11, 13 or 29 atoms in the chain.

41. The conjugate of claim 1, wherein the linker comprises straight alkelene chain containing from 1 up to 50 carbon atoms in the chain.

42. The conjugate of claim 41, wherein the linker comprises 2, 3, 4, 5, 6, 7, 8, 9, 10, 11 or 12 carbon atoms in the alkelene chain.

43. The conjugate of claim 42, wherein the linker comprises 3, 4, 5, 6, 7, 8 or 9 carbon atoms in the alkelene chain.

44. The conjugate of claim 1 having formula
(D)-(L)-(S), or a derivative thereof, wherein
D is a drug moiety; L is a non-releasable linker; and S is a substrate for a protein kinase or a lipid kinase.

45. The conjugate of claim 44 having formula
(D)-(L)-(Sp), or a derivative thereof, wherein
D is a drug moiety; L is a non-releasable linker; and Sp is a peptide substrate containing 3-25 amino acids selected from natural and non-natural amino acids.

46. The conjugate of claim 45, wherein the peptide substrate is attached to the drug moiety via a carboxy-terminus or N-terminus of the peptide.

47. The conjugate of claim 46, wherein the peptide substrate is attached to the drug moiety via the carboxy-terminus of the peptide.

48. The conjugate of claim 46, wherein the N-terminus of the peptide is free or is capped with a capping group.

49. The conjugate of claim 48, wherein the N-terminus of the peptide is free.

50. The conjugate of claim 48, wherein the N-terminus of the peptide is capped with a capping group.

51. The conjugate of claim 48, wherein the capping group is selected from acetyl, benzoyl, pivaloyl, CBz and BOC.

52. The conjugate of claim 48, wherein the peptide substrate comprises one or more amino acids with a reactive group in the amino acid side chain.

53. The conjugate of claim 52, wherein the amino acid is selected from lysine, aspartic acid and glutamic acid.

54. The conjugate of claim 52, wherein the reactive group is optionally capped with capping group.

55. The conjugate of claim 54, wherein the capping group is selected from acetyl, benzoyl, pivaloyl, CBz, BOC, t-butyl and DMAB.

56. The conjugate of claim 45, wherein the peptide substrate contains at least one amino acid selected from tyrosine, threonine, serine, glycine, glutamic acid, proline and arginine.

57. The conjugate of claim 45, wherein the peptide substrate contains at least one amino acid selected from tyrosine, threonine and serine.

58. The conjugate of claim 45, wherein the peptide substrate contains at least one tyrosine.

59. The conjugate of claim 45, wherein the peptide substrate contains at least one serine.

60. The conjugate of claim 45, wherein the peptide substrate contains at least one threonine.

61. The conjugate of claim 45, wherein the substrate comprises:
(Xaa)ₙ₁-Zaa-(Xaa)ₘ₁
wherein Zaa is a non-degenerate phosphorylatable amino acid selected from a group consisting of Ser, Thr and Tyr; Xaa is any amino acid; and n1 and m1 are integers selected from 1-10.

62. The conjugate of claim 61, wherein Zaa is Ser or Thr, and Xaa is any amino acid except Ser or Thr.

63. The conjugate of claim 61, wherein Zaa is Tyr and Xaa is any amino acid except Tyr.

64. The conjugate of claim 61, wherein Zaa is a non-degenerate phosphorylatable amino acid selected from Ser and Thr and Xaa is any amino acid except Ser and Thr.

65. The conjugate of claim 61, wherein Zaa is Tyr and Xaa is any amino acid except Tyr.

66. The conjugate of claim 1, wherein the substrate comprises:
Xaa1-Xaa2-Xaa3-Xaa4-Xaa5-Xaa6-Xaa7-Xaa8-Xaa9;
wherein Xaa7 is selected from serine, D-serine and threonine;
Xaa6 is selected from serine, lysine, arginine, tyrosine, glutamic acid and phenylalanine;
Xaa5 is selected from serine, threonine, tyrosine, alanine and lysine;
Xaa4 is arginine;
Xaa3 is any amino acid;
Xaa2 is arginine;
Xaa1 is glycine, arginine, lysine, phenylalanine, proline or serine;
Xaa8 is phenylalanine, arginine, valine or tyrosine; and
Xaa9 is serine, glycine, alanine, proline, threonine, glutamic acid or glutamine.

67. The conjugate of claim 66, wherein the substrate comprises:
(Xaa0)p-(Xaa1)q-Xaa2-Xaa3-Xaa4-Xaa5-Xaa6-Xaa7-Xaa8-(Xaa9)r-(Xaa10)s-(Xaa11)t
where p,q and r are each independently 0 or 1;
Xaa0 is glycine, arginine, lysine, phenylalanine, proline or serine;
Xaa10 is glutamic acid; and
Xaa11 is glycine.

68. The conjugate of claim 66, wherein Xaa7 is serine or D-serine.

69. The conjugate of claim 66, wherein Xaa6 is selected from serine, lysine, glutamic acid, arginine, tyrosine and phenylalanine.

70. The conjugate of claim 66, wherein Xaa6 is serine or glutamic acid.

71. The conjugate of claim 66, wherein Xaa6 is serine.

72. The conjugate of claim 66, wherein Xaa5 is selected from serine, threonine, tyrosine, alanine and lysine.

73. The conjugate of claim 66, wherein Xaa5 is threonine or lysine. In certain embodiments, Xaa5 is threonine.

74. The conjugate of claim 66, wherein Xaa3 is proline or serine.

75. The conjugate of claim 66, wherein Xaa1 is glycine or arginine.

76. The conjugate of claim 66, wherein Xaa8 is phenylalanine or tyrosine.

77. The conjugate of claim 66, wherein Xaa8 is phenylalanine.

78. The conjugate of claim 66, wherein Xaa9 is serine, glycine, alanine, proline, threonine, glutamic acid or glutamine.

79. The conjugate of claim 66, wherein Xaa9 is alanine.

80. The conjugate of claim 67, wherein Xaa10 is glutamic acid.

81. The conjugate of claim 67, wherein Xaa11 is glycine.

82. The conjugate of claim 1, wherein the substrate comprises:

| | |
|---|---|
| Gly-Arg-Pro-Arg-Thr-Ser-Ser- | (SEQ ID NO. 5) |
| Phe-Ala-Glu-Gly; | |
| Gly-Arg-Pro-Arg-Thr-Ser-DSer- | (SEQ ID NO. 1406) |
| Phe-Ala-Glu-Gly; | |
| Gly-Arg-Pro-Arg-Ala-Ala-Ala-Phe- | (SEQ ID NO. 1407) |
| Ala-Glu-Gly; | |
| Arg-Ser-Arg-Thr-Ser-Ser-Phe-Ala- | (SEQ ID NO. 1408) |
| Glu-Gly; | |
| Gly-Arg-Ser-Arg-Thr-Ser-Ser-Phe- | (SEQ ID NO. 1409) |
| Ala-Glu-Gly; | |
| Arg-Pro-Arg-Thr-Ser-Ser-Phe; | (SEQ ID NO. 6) |
| Arg-Ser-Arg-Thr-Ser-Ser-Phe | (SEQ ID NO. 1410) |
| and | |
| Arg-Pro-Arg-Lys-Glu-Ser-Tyr. | (SEQ ID NO. 1411) |

83. The conjugate of claim 82, wherein the peptide substrate is

| | |
|---|---|
| Gly-Arg-Pro-Arg-Thr-Ser-Ser-Phe-Ala- | (SEQ ID NO. 5) |
| Glu-Gly; | |

84. The conjugate of claim 82, wherein the peptide substrate comprises an N-terminal amino acid that has a free amino group.

85. The conjugate of claim 82, wherein the peptide substrate comprises an N-terminal capping group selected from an acetyl, benzoyl, pivaloyl, CBz and BOC.

86. The conjugate of claim 82, wherein the capping group is a pivaloyl.

87. The conjugate of claim 82, wherein the capping group is a benzoyl.

88. The conjugate of claim 45, wherein the peptide substrate comprises:
(P1)ₐ-P2-P3-P4-P5-(P6)_{b}-(P7)_{c},
wherein a, b and c are each independently 0 or 1;
P1 is selected from tyrosine, phenylalanine, tryptophan, tyrosine, tryptophan and serine;
P2 is selected from isoleucine, leucine and valine;
P3 is tyrosine or D-tyrosine;
P4 is glycine; serine or alanine;
P5 is serine, threonine, alanine, valine, glycine, tyrosine or lysine;
P6 is phenylalanine, tyrosine, D-phenylalanine, D-tyrosine or N-methylphenylalanine; and
P7 is lysine, arginine, serine, histidine, D-lysine, 2,4-diamino-n-butyric acid, 2,3-diaminopropionic acid or ornithine.

89. The conjugate of claim 88, wherein P1 is selected from tyrosine, phenylalanine, tryptophan and tyrosine.

90. The conjugate of claim 88, wherein P1 is tyrosine.

91. The conjugate of claim 88, wherein P2 is selected from isoleucine, leucine and valine.

92. The conjugate of claim 88, wherein P2 is isoleucine.

93. The conjugate of claim 88, wherein P3 is tyrosine.

94. The conjugate of claim 88, wherein P4 is glycine.

95. The conjugate of claim 88, wherein P5 is serine, threonine or alanine.

96. The conjugate of claim 88, wherein P5 is serine.

97. The conjugate of claim 88, wherein P6 is phenylalanine or tyrosine.

98. The conjugate of claim 88, wherein P7 is lysine, Dab, Dap or ornithine.

99. The conjugate of claim 88, wherein P7 is lysine.

100. The conjugate of claim 88, wherein
P2 is selected from isoleucine, leucine and valine;
P3 is tyrosine;
P4 is Glycine; and
P5 is serine, threonine or alanine.

101. The conjugate of claim 88, wherein
P2 is selected from isoleucine, leucine and valine; and
P5 is serine, threonine or alanine.

102. The conjugate of claim 88, wherein P2 is isoleucine, P3 is tyrosine, P4 is glycine and P5 is serine.

103. The conjugate of claim 88, wherein P3 is tyrosine, and P4 is glycine.

104. The conjugate of claim 88, wherein the peptide substrate comprises
(P0)ₐ₁(P1)ₐ-P2-P3-P4-P5-(P6)_{b}-(P7)_{c},
where a1 is 0 or 1 and P0 is glutamic acid.

105. The conjugate of claim 45, wherein the peptide substrate comprises:

| | |
|---|---|
| Tyr-Ile-Tyr-Gly-Ser-Phe-Lys; | (SEQ ID NO. 668) |
| Glu-Tyr-Ile-Tyr-Gly-Ser-Phe-Lys: | (SEQ ID NO. 1412) |
| Tyr-Ile-Tyr-Gly-Ser-Phe-Arg; | (SEQ ID NO. 1413) |
| Tyr-Ile-DTyr-Gly-Ser-Phe-Arg; | (SEQ ID NO. 1414) |
| Tyr-Ile-Phe-Gly-Ser-Phe-Arg | (SEQ ID NO. 1415) |
| Glu-Tyr-Ile-Tyr-Gly-Ser-Phe-Lys; | (SEQ ID NO. 1416) |
| Glu-Tyr-Ile-Tyr-Gly-Ser-Phe-Arg; | (SEQ ID NO. 1417) |
| Tyr-Ile-Tyr-Gly-Ser-Phe-Ser; | (SEQ ID NO. 1418) |
| Tyr-Ile-Tyr-Gly-Ser-Phe-His | (SEQ ID NO. 1419) |
| or | |
| Gly-Ile-Lys-Trp-His-His-Tyr. | (SEQ ID NO. 1420) |

106. The conjugate of claim 105, wherein the peptide substrate is

| | |
|---|---|
| Tyr-Ile-Tyr-Gly-Ser-Phe-Arg; | (SEQ ID NO. 1413) |
| Glu-Tyr-Ile-Tyr-Gly-Ser-Phe-Lys; | (SEQ ID NO. 1412) |
| or | |
| Tyr-Ile-Tyr-Gly-Ser-Phe-Lys. | (SEQ ID NO. 668) |

107. The conjugate of claim 105, wherein the peptide substrate comprises an N-terminal amino acid with a free amino group.

108. The conjugate of claim 105, wherein the peptide substrate comprises an N-terminal amino acid capped with a capping group selected from an acetyl, benzoyl, pivaloyl, CBz and BOC.

109. The conjugate of claim 108, wherein the capping group selected from acetyl, pivaloyl and CBz.

110. The conjugate of claim 1 having formula
(D)-(L)-(SI), or a derivative thereof, wherein
D is a drug moiety; L is a non-releasable linker; and S1 is a substrate for a lipid kinase.

111. The conjugate of claim 110, wherein the lipid kinase is a sphingosine kinase.

112. The conjugate of claim 110, wherein, the substrate is selected from: 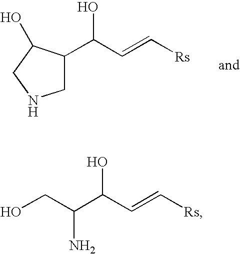
where Rs is alkyl or aryl.

113. The conjugate of claim 112, wherein Rs is alkyl.

114. The conjugate of claim 112, wherein the substrate has formula: 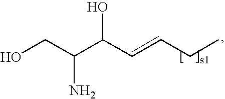
where s1 is 3-20.

115. The conjugate of claim 110, wherein the substrate for the lipid kinase is selected from sphingosine, sphingenine, 1-O-hexadecyl-2-desoxy -2-amino-sn-glycerol, 1-hexadecanol, N-acetyl-D-erythro-sphingenine, 1-amino-2-octadecanol, 2-amino-1-hexadecanol, α-monooleoyl-glycerol, 1-O-octadecyl-rac-glycerol, 1-O-octadecyl-sn-glycerol, and 3-O-octadecyl-sn-glycerol.

116. The conjugate of claim 115, wherein the substrate is sphingosine.

117. The conjugate of claim 112, wherein the substrate has formula: 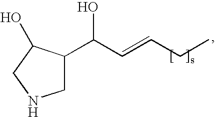
where s is 3-20.

118. The conjugate of claim 117, wherein the substrate has formula selected from:

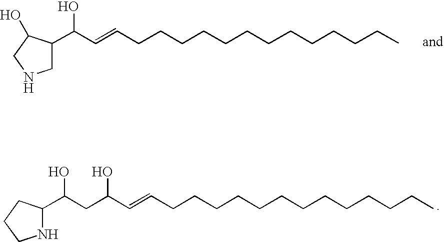

120. The conjugate of claim 45 having formula: 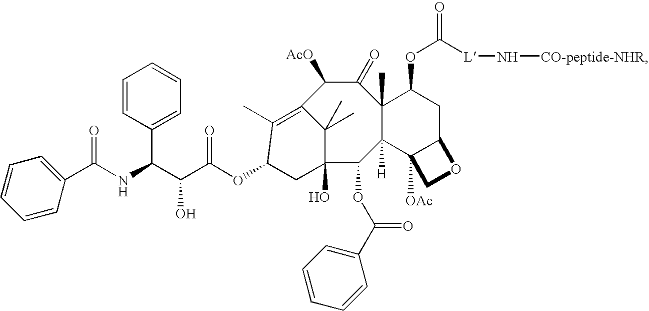
wherein R is a capping group selected from acetyl, benzoyl, pivaloyl, CBz and BOC.

121. The conjugate of claim 45 having formula: 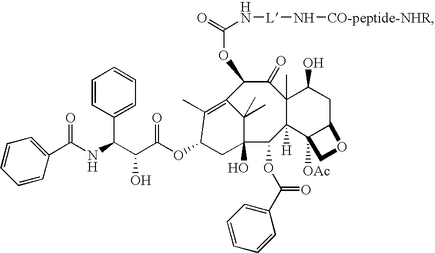
wherein R is a capping group selected from acetyl, benzoyl, pivaloyl, CBz and BOC.

122. The conjugate of claim 45 having formula: 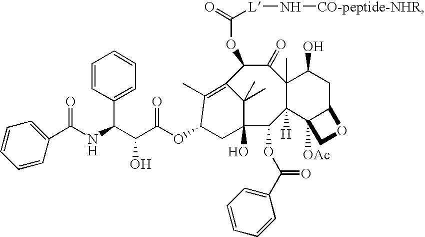
wherein R is a capping group selected from acetyl, benzoyl, pivaloyl, CBz and BOC.

123. The conjugate of claim 45 having formula: 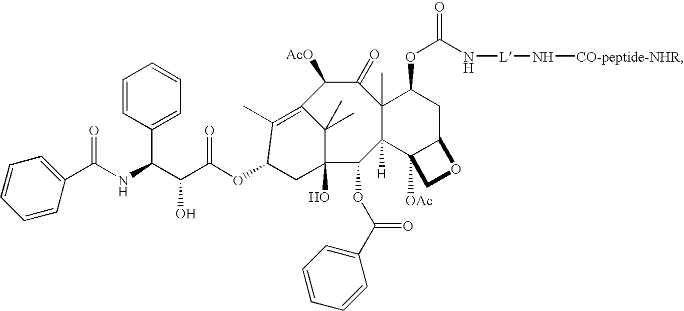
wherein R is a capping group selected from acetyl, benzoyl, pivaloyl, CBz and BOC.

124. The conjugate of claim 45 having formula: 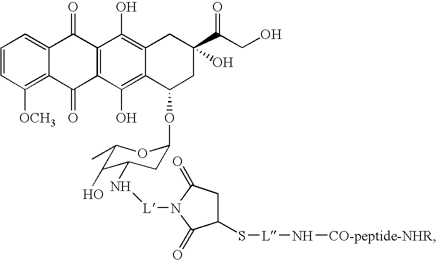
wherein L′ and L″ are each independently alkyl linker or PEG linker and R is a capping group selected from acetyl, benzoyl, pivaloyl, CBz and BOC.

125. The conjugate of claim 45 having formula: 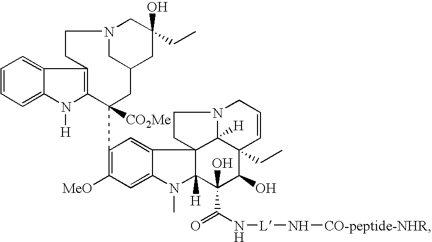
wherein R is a capping group selected from acetyl, benzoyl, pivaloyl, CBz and BOC.

126. The conjugate of claim 110 having formula: 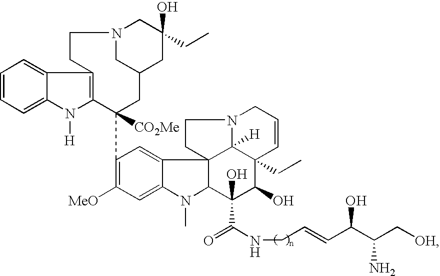
where n is 2-10.

127. The conjugate of claim 110 having formula: 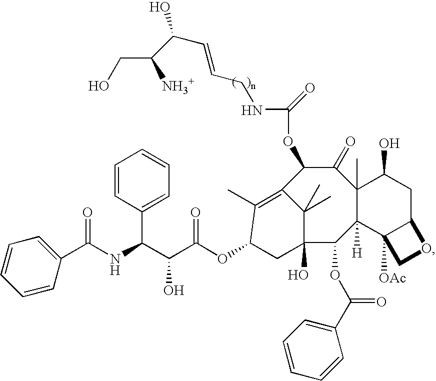
where n is 2-10.

128. The conjugate of claim 110 having formula: 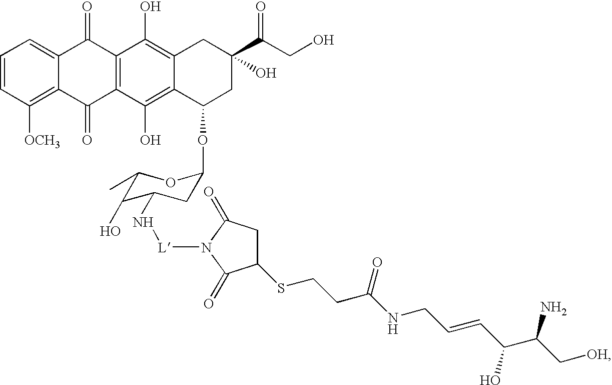
Where n is 2-10.

129. The conjugate of claim 1, wherein the conjugate has an improved cytotoxic selectivity index as compared to an unconjugated drug.

130. The conjugate of claim 1, wherein the cytotoxic selectivity index is about 1.5 folds up to more than about 100 folds improved.

131. The conjugate of claim 1 selected from Table 6.

132. The conjugate of claim 1 selected from

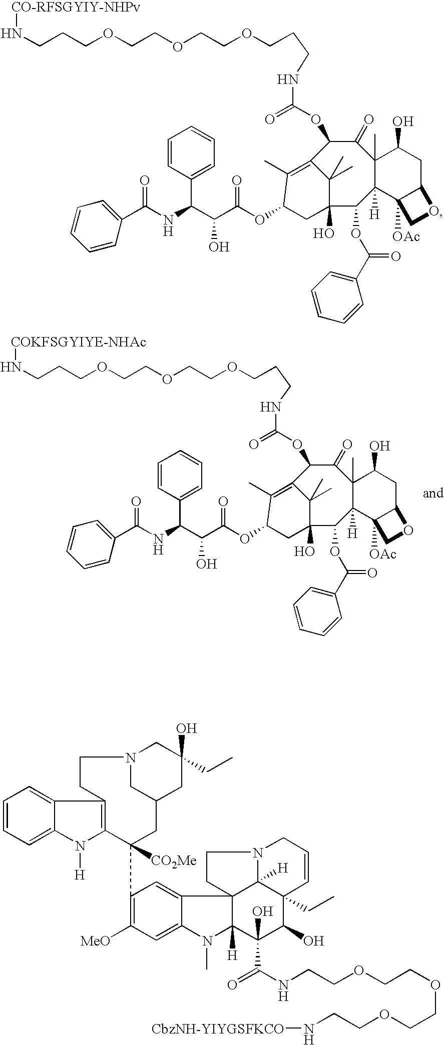

133. A pharmaceutical composition comprising the conjugate of claim 1 in a pharmaceutically acceptable carrier.

134. An article of manufacture, comprising packaging material, the conjugate of claim 1, or a pharmaceutically acceptable derivative thereof, contained within packaging material, which is used for treatment, prevention or amelioration of one or more symptoms associated with ACAMPS, and a label that indicates that the compound or pharmaceutically acceptable derivative thereof is used for treatment, prevention or amelioration of one or more symptoms associated with ACAMPS.

135. A peptide comprising an amino acid sequence:

| | |
|---|---|
| Gly-Arg-Pro-Arg-Thr-Ser-Ser- | (SEQ ID NO. 5) |
| Phe-Ala-Glu-Gly; | |
| Gly-Arg-Pro-Arg-Thr-Ser-DSer- | (SEQ ID NO. 1406) |
| Phe-Ala-Glu-Gly; | |
| Gly-Arg-Pro-Arg-Ala-Ala-Ala-Phe- | (SEQ ID NO. 1407) |
| Ala-Glu-Gly; | |
| Arg-Ser-Arg-Thr-Ser-Ser-Phe-Ala- | (SEQ ID NO. 1408) |
| Glu-Gly; | |
| Gly-Arg-Ser-Arg-Thr-Ser-Ser-Phe- | (SEQ ID NO. 1409) |
| Ala-Glu-Gly; | |
| Arg-Pro-Arg-Thr-Ser-Ser-Phe; | (SEQ ID NO. 6) |
| Arg-Ser-Arg-Thr-Ser-Ser-Phe | (SEQ ID NO. 1410) |
| and | |
| Arg-Pro-Arg-Lys-Glu-Ser-Tyr, | (SEQ ID NO. 1411) |

wherein the peptide is free from resin.

136. The peptide of claim 135, wherein the amino acid at N terminus is capped with a capping group.

137. The peptide of claim 136, wherein the capping group is selected from acetyl, pivaloyl, benzoyl, Boc and CBz.

138. The peptide of claim 137, wherein the capping group is selected from acetyl and pivaloyl.

139. The peptide of claim 111, wherein the peptide is a substrate for Akt kinase.

140. A peptide comprising an amino acid sequence:

| | |
|---|---|
| Tyr-Ile-Tyr-Gly-Ser-Phe-Lys; | (SEQ ID NO. 668) |
| Glu-Tyr-Ile-Tyr-Gly-Ser-Phe-Lys: | (SEQ ID NO. 1412) |
| Tyr-Ile-Tyr-Gly-Ser-Phe-Arg; | (SEQ ID NO. 1413) |
| Tyr-Ile-DTyr-Gly-Ser-Phe-Arg; | (SEQ ID NO. 1414) |
| Tyr-Ile-Phe-Gly-Ser-Phe-Arg | (SEQ ID NO. 1415) |
| Glu-Tyr-Ile-Tyr-Gly-Ser-Phe-Lys; | (SEQ ID NO. 1416) |
| Glu-Tyr-Ile-Tyr-Gly-Ser-Phe-Arg; | (SEQ ID NO. 1417) |
| Tyr-Ile-Tyr-Gly-Ser-Phe-Ser; | (SEQ ID NO. 1418) |
| Tyr-Ile-Tyr-Gly-Ser-Phe-His | (SEQ ID NO. 1419) |
| and | |
| Gly-Ile-Lys-Trp-His-His-Tyr. | (SEQ ID NO. 1420) |

wherein the peptide is free from resin, with the proviso that when the peptide is Tyr-Ile-Tyr-Gly-Ser-Phe-Lys (SEQ ID NO. 668), then the N terminus is capped with a capping group.

141. The peptide of claim 140, wherein the amino acid at N terminus is capped with a capping group.

142. The peptide of claim 140 selected from:

| | |
|---|---|
| Tyr-Ile-Tyr-Gly-Ser-Phe-Arg; | (SEQ ID NO. 1413) |
| Glu-Tyr-Ile-Tyr-Gly-Ser-Phe-Lys; | (SEQ ID NO. 1412) |
| and | |
| Tyr-Ile-Tyr-Gly-Ser-Phe-Lys. | (SEQ ID NO. 668) |

143. The peptide of claim 141, wherein the capping group is selected from acetyl, pivaloyl, benzoyl, Boc and CBz.

144. The peptide of claim 143, wherein the capping group is selected from acetyl and pivaloyl.

145. The peptide of claim 115, wherein the peptide is a substrate for Src kinase.

146. The conjugate of claim 1, wherein the conjugate has formula: 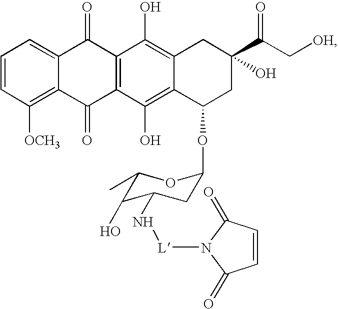
wherein L′ is the non-releasable linker.
